(19) United States
(12) Patent Application Publication
(10) Pub. No.: US 2026/0341411 A1
(43) Pub. Date: Oct. 29, 2026

(54) SYSTEMS AND METHODS FOR COLOCALIZATION

(71) Applicant: 10x Genomics, Inc., Pleasanton, CA (US)

(72) Inventor: Ace George Santiago, Pleasanton, CA (US)

(21) Appl. No.: 19/101,712

(22) PCT Filed: Aug. 9, 2023
(86) PCT No.: PCT/US23/71901

§ 371 Date: Feb. 6, 2025

Related U.S. Application Data
(60) Provisional application No. 63/396,902, filed on Aug. 10, 2022.

(51) Int. Cl. G16B 35/20; C12N 15/10; G06T 7/00; G06T 7/136; G06V 10/26; G06V 10/28; G06V 20/69

(52) U.S. Cl. CPC G16B 35/20; C12N 15/1065; G06T 7/0012; G06T 7/136; G06V 10/26; G06V 10/28; G06V 20/695; G06T 2207/30024; G06V 2201/04

(57) ABSTRACT

A method for colocalization obtains a first image encoding spatial abundance data for a nucleic acid from a biological sample through pixel values of subset of pixels in the first image. A second image is obtained encoding spatial abundance data for a non-nucleic acid analyte from the sample through pixel intensity values of subset of pixels in the second image. Thresholds of the first and second image are obtained. A determination, using a registration between the first and second images, of a first summation of the value of each pixel in the subset of pixels of the first image in which the corresponding pixel in the second image exceeds the second threshold value is made. The first summation, normalized to a summation of each value of each pixel in the subset of pixels of the first image, provides a measure of colocalization between the nucleic acid and non-nucleic acid analyte.

### CROSS REFERENCE TO RELATED PATENT APPLICATIONS

The present application claims priority to U.S. Provisional Patent Application No. 63/396,902, entitled “SYSTEMS AND METHODS FOR COLOCALIZATION,” filed Aug. 10, 2022, which is hereby incorporated by reference.

### TECHNICAL FIELD

This specification describes technologies relating to measures of colocalization between (i) the unique molecular identifier (UMI) spatial analyte abundance data for a nucleic acid analyte and (ii) the spatial analyte UMI abundance data for a non-nucleic acid analyte.

### BACKGROUND

Spatial resolution of analytes in tissues provides new insights into the processes underlying biological function and morphology, such as cell fate and development, disease progression and detection, and cellular and tissue-level regulatory networks. See, Satija et al., 2015, “Spatial reconstruction of single-cell gene expression data,” Nature Biotechnology. 33, 495-502, doi:10.1038.nbt.3192 and Achim et al., 2015, “High-throughput spatial mapping of single-cell RNA-seq data to tissue of origin,” Nature Biotechnology 33:503-509, doi:10.1038/nbt.3209, each of which is hereby incorporated herein by reference in its entirety. An understanding of the spatial patterns or other forms of relationships between analytes can provide information on differential cell behavior. This, in turn, can help to elucidate complex conditions such as diseases. For example, the determination that the abundance of an analyte (e.g., a gene or a protein) is associated with a tissue subpopulation of a particular tissue class (e.g., disease tissue, healthy tissue, the boundary of disease and healthy tissue, etc.) provides inferential evidence of the association of the analyte with a condition such as complex disease. Likewise, the determination that the abundance of an analyte is associated with a particular subpopulation of a heterogeneous cell population in a complex 2-dimensional or 3-dimensional tissue (e.g., a mammalian brain, liver, kidney, heart, a tumor, or a developing embryo of a model organism) provides inferential evidence of the association of the analyte in the particular subpopulation.

Thus, spatial analysis of analytes can, among other practical applications, provide information for the early detection of disease by identifying at-risk regions in complex tissues and characterizing the analyte profiles present in these regions through spatial reconstruction (e.g., of gene expression, protein expression, DNA methylation, and/or single nucleotide polymorphisms, among others).

A high-resolution spatial mapping of analytes to their specific location within a region or subregion reveals spatial expression patterns of analytes, provides relational data, and further implicates analyte network interactions relating to disease or other morphologies or phenotypes of interest, resulting in a holistic understanding of cells in their morphological context. See, 10×, 2019, “Spatially-Resolved Transcriptomics,” 10×, 2019, “Inside Visium Spatial Technology,” and 10×, 2019, “Visium Spatial Gene Expression Solution,” each of which is hereby incorporated herein by reference in its entirety. Such spatial resolution technology makes use of a combination of a unique molecular identifier (UMI) and a spatial barcode for each analyte. The spatial barcode resolves an analyte to a particular location on an arrayed substrate, and thus to a corresponding location in a tissue overlayed on the arrayed substrate, while the UMI provides the ability to count the number of instances of a given analyte. The spatial mapping of a given analyte, then, is a spatial mapping of the UMI count of the analyte making use of the location of the associated spatial barcodes.

Of even further interest is comparison of the spatial distribution of related analytes in a tissue, for instance, whether they co-localize. In one such approach, one of the analytes is a nucleic acid of a particular gene and the other analyte is the gene product (e.g., protein). Each of the nucleic analytes and each of the non-nucleic acid analytes is indexed by UMIs and spatial barcodes. In some instances, a spatial distribution comparison between the nucleic acid analyte and the non-nucleic acid analyte involves a comparison of the spatial distribution of the UMI count for the nucleic acid analyte versus the spatial distribution of the UMI count for the non-nucleic analyte. However, in some embodiments, the spatial distribution of the non-nucleic analyte is not based on UMI count but rather is based on fluorescence of a marker associated with the non-nucleic acid analyte. Such spatial distribution comparisons can be used to understand the function of genes and their protein products.

Different assays are used to produce the spatial data for nucleic acid based analytes versus the spatial data for non-nucleic analytes. Thus, the spatial data (UMI count and spatial location) for nucleic acid based analytes versus the spatial data for non-nucleic analytes are typically on different images, which are then registered to each other and analyzed to quantify the extent to which a particular nucleic acid and non-nucleic acid colocalize.

Colocalization can be described as consisting of two components: co-occurrence, that is the spatial overlap of two analytes, and correlation, in which two analytes not only overlap with one another but codistribute in proportion to one another spatially. One statistic for quantifying colocalization is the Pearson's correlation coefficient (PCC):

$$PCC = \frac{{\sum }_{i} ( {R}_{i} - \overline{R} ) \times ( {G}_{i} - \overline{G} )}{\sqrt{{\sum }_{i} {( {R}_{i} - \overline{R} )}^{2} \times {( {G}_{i} - \overline{G} )}^{2}}}$$

where Rᵢ is the UMI count of a first analyte (e.g., a nucleic acid analyte) at position i in a first image, Gᵢ refers to an abundance of a second analyte (e.g., a non-nucleic analyte such as a protein, in the form of a UMI count or a count of a marker associated with the non-nucleic analyte) at corresponding position i in a second image, and R̅ and G̅ refer to the mean counts of the first and second analytes, respectively, across all the positions in the entire first and second images. In some instances, each position i is a pixel, whereas in other instances each position i is a collection of pixels that represent a corresponding spot on an array. PCC values range from 1 for two images whose spatial distributions for the first and second analytes are perfectly, linearly related, to −1 for two images whose spatial distributions for the first and second analytes are perfectly, but inversely, related to one another. Values near zero reflect spatial distributions that are uncorrelated with one another. One drawback with PCC values is that the negative values possible with PCC can be difficult to interpret. Another drawback with PCC values is that two analytes that have excellent overlap may still have a low PCC value. This problem is illustrated in FIG. 3. On the left side of FIG. 3 the spatial distribution of two analytes is illustrated, in which the spatial intensity is similar and so is the co-localization. This results in a high PCC value. On the right side of FIG. 3 the spatial distribution of two analytes is illustrated in which the co-localization remains similar. However, the signal intensity is heterogeneous for one of the analytes resulting in a low PCC value even though the co-localization of the two analytes remains high.

Thus, there is a need in the art for systems and methods that provide improved analysis of spatial analyte data, particularly by using co-localization metrics that are insensitive to heterogeneity in signal intensity between two analytes whose spatial distributions are being compared.

### SUMMARY

Technical solutions (e.g., computing systems, methods, and non-transitory computer readable storage mediums) for addressing the above-identified problems in analysis of spatial analyte data are provided in the present disclosure. In particular, various methods of quantifying the spatial unique molecular identifier distribution of analytes are described herein.

It should be understood that this summary is not an extensive overview of the present disclosure. It is not intended to identify key/critical elements of the present disclosure or to delineate the scope of the present disclosure. Its sole purpose is to present some of the aspects of the present disclosure in a simplified form as a prelude to the more detailed description that is presented later.

One aspect of the present disclosure provides a method for colocalization of two analytes in a biological sample, the method comprising using a computer system comprising one or more processing cores and a memory. There is obtained, in electronic form, a first two-dimensional image exceeding 100 kilobytes in size, comprising a first plurality of pixels, and encoding spatial analyte unique molecular identifier (UMI) abundance data for a nucleic acid analyte from a biological sample of a subject through pixel intensity values across a first subset of pixels in the first plurality of pixels. The first subset of pixels in the first plurality of pixels comprises 500 pixels or 1000 pixels.

Further in the methods, there is obtained, in electronic form, a second two-dimensional image exceeding 100 kilobytes in size, comprising a second plurality of pixels, and encoding spatial analyte abundance data for a non-nucleic acid analyte from the biological sample of a subject through pixel intensity values across a first subset of pixels in the second plurality of pixels. The first subset of the second plurality of pixels comprises 500 pixels and a registration between the first and second image is determined.

Further in the method, a first threshold value of the first image is obtained.

Further in the method, a second threshold value of the second image is obtained.

Further in the method a determination is made, using the registration, of a first summation of the respective UMI abundance value of each respective pixel in the first subset of the first plurality of pixels in which the corresponding pixel in the first subset of the second plurality of pixels exceeds the second threshold value. This first summation is normalized to a second summation of each respective UMI abundance value of each pixel in the first subset of the first plurality of pixels and provided as a first measure of colocalization between (i) the spatial analyte UMI abundance data for the nucleic acid analyte and (ii) the spatial analyte abundance data for the non-nucleic acid analyte.

In some embodiments, the method further comprises determining, using the registration, a third summation of the respective abundance value of each respective pixel in the first subset of the second plurality of pixels in which the corresponding pixel in the first subset of the first plurality of pixels exceeds the first threshold value. The third summation is normalized to a fourth summation of each respective abundance value of each pixel in the first subset of the second plurality of pixels, as a second measure of colocalization between (i) the spatial analyte UMI abundance data for the nucleic acid analyte and (ii) the spatial analyte abundance data for the non-nucleic acid analyte.

In some embodiments, the nucleic acid analyte is a plurality of RNA or DNA molecules, arising from the biological sample of the subject, where each RNA or DNA molecule in the plurality of RNA or DNA molecules encodes all or a portion of a first gene, and the spatial analyte UMI abundance data for the nucleic acid analyte quantifies UMI abundance of a plurality of spatially barcoded sequence reads of the plurality of RNA or DNA molecules.

In some such embodiments, the non-nucleic acid analyte is a first protein and the spatial analyte abundance data for the non-nucleic acid analyte quantifies UMI abundance of a labeled antibody to the first protein. In some such embodiments, the first gene encodes the first protein.

In some embodiments, the method further comprises segmenting the first image to identify the first subset of the first plurality of pixels using a first segmentation algorithm and segmenting the second image to identify the first subset of the second plurality of pixels using a second segmentation algorithm.

In some such embodiments, the first segmentation algorithm and the second segmentation algorithm is a binarization method using global thresholding.

In alternative embodiments, the first segmentation algorithm identifies the first threshold value as a first pixel intensity value that that divides the pixels of the first image into either the first subset of the first plurality of pixels or a second subset of the first plurality of pixels, where the first threshold value represents a minimization of intra-class intensity variance between the first and second subset of the first plurality of pixels or a maximization of inter-class variance between the first and second subset of the first plurality of pixels.

In some embodiments, the method further comprises determining a Pearson's correlation coefficient using (i) the intensity of each pixel in the first subset of the first plurality of pixels, (ii) the intensity of each pixel in the second image corresponding to a pixel in the first subset of the first plurality of pixels, (iii) a first mean value across the first subset of the first plurality of pixels, and (iv) a second mean value across the pixels in the second image corresponding to the first subset of pixels in the first plurality of pixels. In such embodiments, the Pearson's correlation coefficient is provided as a third measure of colocalization between (i) the spatial analyte UMI abundance data for the nucleic acid analyte and (ii) the spatial analyte abundance data for the non-nucleic acid analyte.

In some embodiments, the method further comprises determining a ratio between a number of pixels in the first subset of the first plurality of pixels and a number of pixels in the first subset of the second plurality of pixels. This ratio is provided as an indication of a size comparison between (i) the spatial analyte UMI abundance data for the nucleic acid analyte and (ii) the spatial analyte abundance data for the non-nucleic acid analyte.

In some embodiments, the method further comprises displaying the first image on a display in electronic communication with the computer system, obtaining user selection of a first region of the first image, where each pixel in the first subset of pixels in the first plurality of pixels is within the first region, and removing from the first image all pixels outside the first region prior to obtaining the threshold value of the first image. In some such embodiments, the method further comprises defining a second region in the second image, where the second region includes a corresponding pixel for each pixel in the first subset of the first plurality of images, and each pixel in the second region in the second image corresponds to a pixel in the first region of the first image in accordance with the registration. In such embodiments, each pixel in the first subset of pixels in the second plurality of pixels is within the second region and the method further comprises removing from the second image each pixel outside the second region prior to obtaining the threshold value of the second image.

In some embodiments, the method further comprises removing, from the first subset of the first plurality of pixels, clusters of pixels within the first image that constitute less than a threshold number of pixels and removing, from the first subset of the second plurality of pixels, clusters of pixels within the second image that constitute less than a threshold number of pixels. In some such embodiments, the threshold number of pixels is 5, 6, 7, 8, 9, 10, 11, 12, 13, 14, 15, 16, 17, 18, 19, 20, or between 20 and 200 pixels.

In some embodiments, the first subset of the first plurality of pixels occupies a first plurality of disjoint areas within the first image and the first subset of the second plurality of pixels occupies a second plurality of disjoint areas within the second image. In some such embodiments, the first plurality of disjoint areas comprises 5, 10, 15, or 20 disjoint areas and the second plurality of disjoint areas comprises 5, 10, 15, or 20 disjoint areas.

In some embodiments, the intensity of the first plurality of pixels and the second plurality of pixels is on a log₂ based scale relative to measured intensity. For instance, in some embodiments, to extract the signal from the tissue, the first and second images were transformed from the RGB image space to the HSV image space. Specifically, the locations of the pixels with nonzero counts on the RGB space were extracted from the hue channel of the HSV image space.

In some embodiments, the method further comprises down sampling the first image to a size of the second image prior to determining the first summation using the registration.

In some embodiments, the method further comprises down sampling the second image to a size of the first image prior to determining the first summation using the registration.

In some embodiments, the second image is obtained by bright-field microscopy, immunohistochemistry, or fluorescence microscopy.

In some embodiments, the biological sample of the subject is prepared for imaging to create the second image using a detectable marker selected from the group consisting of an antibody, a fluorescent label, a radioactive label, a chemiluminescent label, a colorimetric label, a colorimetric label, or a combination thereof.

In some embodiments, the biological sample is prepared for imaging to create the second image using a stain selected from the group consisting of live/dead stain, trypan blue, periodic acid-Schiff reaction stain, Masson's trichrome, Alcian blue, van Gieson, reticulin, Azan, Giemsa, Toluidine blue, isamin blue, sudan black and osmium, acridine orange, Bismarck brown, carmine, Coomassie blue, cresyl violet, DAPI, eosin, ethidium bromide, acid fuchsine, hematoxylin, Hoechst stains, iodine, methyl green, methylene blue, neutral red, Nile blue, Nile red, osmium tetroxide, propidium iodide, rhodamine, safranin, or a combination thereof.

In some embodiments, the first image is obtained by filtering a spatial dataset for UMI abundance data for the nucleic acid analyte, and where the method further comprises obtaining the spatial dataset by a procedure comprising: overlaying the biological sample on a substrate, where the substrate comprises a set of capture spots in the form of an array, obtaining a plurality of sequence reads, in electronic form, from the set of capture spots, where: each respective capture probe plurality in a set of capture probe pluralities is (i) at a different capture spot in the set of capture spots and (ii) directly or indirectly associates with one or more nucleic acid analytes in a plurality of nucleic acid analytes from the biological sample, each respective capture probe plurality in the set of capture probe pluralities is characterized by at least one unique spatial barcode in a plurality of spatial barcodes, the plurality of sequence reads comprises sequence reads corresponding to all or portions of the plurality of nucleic acid analytes, the plurality of sequence reads comprises at least 10,000 sequence reads, and each respective sequence read in the plurality of sequence reads includes a spatial barcode of the corresponding capture probe plurality in the set of capture probe pluralities or a complement thereof; and using all or a subset of the plurality of spatial barcodes to localize respective sequence reads in the plurality of sequence reads to corresponding capture spots in the set of capture spots, thereby dividing the plurality of sequence reads into a plurality of subsets of sequence reads, each respective subset of sequence reads corresponding to a different capture spot in the plurality of capture spots. In some such embodiments, the unique spatial barcode encodes a unique predetermined value selected from the set {1, . . . , 1024}, {1, . . . , 4096}, {1, . . . , 16384}, {1, . . . , 65536}, {1, . . . , 262144}, {1, . . . , 1048576}, {1, . . . , 4194304}, {1, . . . , 16777216}, {1, . . . , 67108864}, or {1, . . . , 1×10¹²}. In some such embodiments, the obtaining the plurality of sequence reads comprises high-throughput sequencing. In some embodiments, a respective capture probe plurality in the set of capture probe pluralities includes 1000 or more capture probes, 2000 or more capture probes, 10,000 or more capture probes, 100,000 or more capture probes, 1×10⁶ or more capture probes, 2×10⁶ or more capture probes, or 5×10⁶ or more capture probes. In some such embodiments, each capture probe in the respective capture probe plurality includes the same spatial barcode from the plurality of spatial barcodes.

In some embodiments, the biological sample is a tissue section.

In some embodiments, the method further comprises using the first measure of colocalization to characterize a biological condition in a subject.

In some embodiments, the first image comprises 10,000 or more pixel values, and the second image comprises 10,000 or more pixel values. In some embodiments, the first image comprises 100,000 or more pixel values, and the second image comprises 100,000 or more pixel values.

Another aspect of the present disclosure provides a computer system comprising one or more processors, memory, and one or more programs, where the one or more programs are stored in the memory and are configured to be executed by the one or more processors. The one or more programs include instructions for performing a method for colocalization comprising obtaining, in electronic form, a first two-dimensional image exceeding 100 kilobytes in size, comprising a first plurality of pixels, and encoding spatial analyte unique molecular identifier (UMI) abundance data for a nucleic acid analyte from a biological sample through pixel intensity values across a first subset of pixels in the first plurality of pixels, where the first subset of the first plurality of pixels comprises 500 pixels. The method further comprises obtaining, in electronic form, a second two-dimensional image exceeding 100 kilobytes in size, comprising a second plurality of pixels, and encoding spatial analyte abundance data for a non-nucleic acid analyte from the biological sample through pixel intensity values across a first subset of pixels in the second plurality of pixels, where the first subset of the second plurality of pixels comprises 500 pixels and a registration between the first and second image is determined. The method further comprises obtaining a first threshold value of the first image and obtaining a second threshold value of the second image. The method further comprises determining, using the registration, a first summation of the respective UMI abundance value of each respective pixel in the first subset of the first plurality of pixels in which the corresponding pixel in the first subset of the second plurality of pixels exceeds the second threshold value. The method further comprises providing the first summation, normalized to a second summation of each respective UMI abundance value of each pixel in the first subset of the first plurality of pixels, as a first measure of colocalization between (i) the spatial analyte UMI abundance data for the nucleic acid analyte and (ii) the spatial analyte abundance data for the non-nucleic acid analyte.

Another aspect of the present disclosure provides a computer readable storage medium storing one or more programs. The one or more programs comprise instructions, which when executed by an electronic device with one or more processors and a memory cause the electronic device to perform a method for colocalization. The method comprises obtaining, in electronic form, a first two-dimensional image exceeding 100 kilobytes in size, comprising a first plurality of pixels, and encoding spatial analyte unique molecular identifier (UMI) abundance data for a nucleic acid analyte from a biological sample through pixel intensity values across a first subset of pixels in the first plurality of pixels, where the first subset of the first plurality of pixels comprises 500 pixels. The method further comprises obtaining, in electronic form, a second two-dimensional image exceeding 100 kilobytes in size, comprising a second plurality of pixels, and encoding spatial analyte abundance data for a non-nucleic acid analyte from the biological sample through pixel intensity values across a first subset of pixels in the second plurality of pixels, where the first subset of the second plurality of pixels comprises 500 pixels and a registration between the first and second image is determined. The method further comprises obtaining a first threshold value of the first image and obtaining a second threshold value of the second image. The method further comprises determining, using the registration, a first summation of the respective UMI abundance value of each respective pixel in the first subset of the first plurality of pixels in which the corresponding pixel in the first subset of the second plurality of pixels exceeds the second threshold value. The method further comprises providing the first summation, normalized to a second summation of each respective UMI abundance value of each pixel in the first subset of the first plurality of pixels, as a first measure of colocalization between (i) the spatial analyte UMI abundance data for the nucleic acid analyte and (ii) the spatial analyte abundance data for the non-nucleic acid analyte.

Various embodiments of systems, methods, and devices within the scope of the appended claims each have several aspects, no single one of which is solely responsible for the desirable attributes described herein. Without limiting the scope of the appended claims, some prominent features are described herein. After considering this discussion, and particularly after reading the section entitled “Detailed Description” one will understand how the features of various embodiments are used.

### INCORPORATION BY REFERENCE

All publications, patents, patent applications, and information available on the Internet and mentioned in this specification are herein incorporated by reference to the same extent as if each individual publication, patent, patent application, or item of information was specifically and individually indicated to be incorporated by reference. To the extent publications, patents, patent applications, or item of information available on the Internet incorporated by reference contradict the disclosure contained in the specification, the specification is intended to supersede and/or take precedence over any such contradictory material.

### DESCRIPTION OF DRAWINGS

The file of this patent contains at least one drawing executed in color. Copies of this patent with color drawing(s) will be provided by the Office upon request and payment of the necessary fee.

The following drawings illustrate certain embodiments of the features and advantages of this disclosure. These embodiments are not intended to limit the scope of the appended claims in any manner. Like reference symbols throughout the several views of the patent application indicate like elements.

FIG. 1 shows an exemplary system for colocalization of analyte data for a biological sample in accordance with some embodiments of the present disclosure.

FIGS. 2A, 2B, 2C, 2D, 2E, 2F, and 2G illustrate non-limiting methods for colocalization of analyte data for a biological sample in accordance with some embodiments of the present disclosure, in which optional steps are illustrated by dashed line boxes.

FIG. 3 illustrates computations of a Pearson correlation coefficient between (i) the spatial analyte abundance data for a nucleic acid analyte and (ii) the spatial analyte abundance data for a corresponding non-nucleic acid analyte.

FIGS. 4A, 4B, and 4C respectively illustrate three different intensity scenarios between (i) the spatial analyte abundance data for a nucleic acid analyte and (ii) the spatial analyte abundance data for a corresponding non-nucleic acid analyte.

FIG. 5 respectively illustrates computation of overlap metrics that are insensitive to signal proportionality in accordance with some embodiments of the present disclosure.

FIG. 6 illustrates two different intensity scenarios between (i) the spatial analyte abundance data for a nucleic acid analyte and (ii) the spatial analyte abundance data for a corresponding non-nucleic acid analyte.

FIG. 7 illustrates obtaining, in electronic form, a first two-dimensional image exceeding 100 kilobytes in size, comprising a first plurality of pixels, and encoding spatial analyte abundance data for a nucleic acid analyte from a biological sample through pixel intensity values across a first subset of pixels in the first plurality of pixels, where the first subset of pixels in the first plurality of pixels comprises 500 pixels in accordance with an embodiment of the present disclosure.

FIG. 8 illustrates obtaining, in electronic form, a second two-dimensional image exceeding 100 kilobytes in size, comprising a second plurality of pixels, and encoding spatial analyte abundance data for a non-nucleic acid analyte from the biological sample through pixel intensity values across a first subset of pixels in the second plurality of pixels, where the first subset of the second plurality of pixels comprises 500 pixels in accordance with an embodiment of the present disclosure.

FIG. 9 illustrates first and second images after gray scaling, masking, and cleaning in accordance with an embodiment of the present disclosure.

FIG. 10 illustrates summary statistics for eight pairs of images for eight different samples in accordance with an embodiment of the present disclosure.

FIG. 11 illustrates summary statistics for eight pairs of images for eight different samples in accordance with an embodiment of the present disclosure.

FIGS. 12A and 12B illustrate 8 pairs of images for 8 different samples as well as their respective merged images in accordance with an embodiment of the present disclosure.

FIG. 13 illustrates a method of associated a spatial barcode with one or more neighboring cells, such that the spatial barcode identifies the one or more cells, and/or contents of the one or more cells, as associated with a particular spatial location.

FIG. 14 illustrates a method of cleaving the spatially-barcoded capture probes from an array and promoting the spatially-barcoded capture probes towards and/or into or onto a sample.

FIGS. 15A and 15B illustrate exemplary workflows that include preparing a sample on a spatially-barcoded array.

FIG. 16 illustrates another exemplary workflow that utilizes a spatially-barcoded array on a substrate (e.g., chip), where spatially-barcoded capture probes are in areas called capture spots (features).

FIG. 17 illustrates an exemplary workflow where a sample is removed from a spatially-barcoded array and spatially-barcoded capture probes are removed from the array for barcoded analyte amplification and library preparation.

FIG. 18 illustrates a substrate (e.g., a chip) that has a plurality of spatial fiducials and a set of capture spots, in accordance with an embodiment of the present disclosure.

### DETAILED DESCRIPTION

### I. Introduction

This disclosure describes apparatus, systems, methods, for spatial analysis of biological samples using colocalization between spatial analyte unique molecular identifier (UMI) abundance data for nucleic acid analytes and spatial analyte UMI abundance data for non-nucleic acid analytes. This section in particular describes certain general terminology, analytes, sample types, and preparative steps that are referred to in later sections of the disclosure.

Advantageously, information regarding the differences in analyte levels (e.g., gene and/or protein expression) within different cells in a tissue of a mammal can also help physicians select or administer a treatment that will be effective and can allow researchers to identify and elucidate differences in cell morphology and/or cell function in single-cell or multicellular organisms (e.g., a mammal) based on the detected differences in analyte levels within different cells in the tissue. For instance, differences in analyte levels within different cells in a tissue of a mammal can provide information on how tissues (e.g., healthy and diseased tissues) function and/or develop. Differences in analyte levels within different cells in a tissue of a mammal can also provide information on mechanisms of disease pathogenesis, mechanisms of action of therapeutic treatments, and/or drug resistance mechanisms and the development of the same in the tissue. Moreover, differences in the presence or absence of analytes within difference cells in a tissue of a multicellular organism (e.g., a mammal) can provide information on drug resistance mechanisms and the development of the same in a tissue of a multicellular organism. Thus, in some embodiments, spatial analysis of analytes can provide information for the early detection of disease by identifying at-risk regions in complex tissues and characterizing the analyte profiles present in these regions through spatial reconstruction (e.g., of gene expression, protein expression, DNA methylation, and/or single nucleotide polymorphisms, among others).

Spatial analysis of analytes can be performed by capturing analytes and/or analyte capture agents or analyte binding domains and mapping them to known locations (e.g., using barcoded capture probes attached to a substrate) using a reference image indicating the tissues or regions of interest within the tissues that correspond to the known locations. For example, in some implementations of spatial analysis, a sample is prepared (e.g., fresh-frozen tissue is sectioned, placed onto a slide, fixed, and/or stained for imaging). The imaging of the sample provides the reference image to be used for spatial analysis. Analyte detection is then performed using, e.g., analyte or analyte ligand capture via barcoded capture probes, library construction, and/or sequencing. The resulting barcoded analyte data and the reference image can be combined during data visualization for spatial analysis. See, e.g., 10×, 2019, “Inside Visium Spatial Technology.” Non-limiting aspects of spatial analysis methodologies are described herein and in WO 2011/127099, WO 2014/210233, WO 2014/210225, WO 2016/162309, WO 2018/091676, WO 2012/140224, WO 2014/060483, U.S. Pat. Nos. 10,002,316, 9,727,810, 10,640,816, Rodriques et al., Science 363(6434):1463-1467, 2019; WO 2018/045186, Lee et al., Nat. Protoc. 10(3):442-458, 2015; WO 2016/007839, WO 2018/045181, WO 2014/163886, Trejo et al., PLoS ONE 14(2):e0212031, 2019, U.S. Pat. No. 10,913,975, Chen et al., Science 348(6233):aaa6090, 2015, Gao et al., BMC Biol. 15:50, 2017, WO 2017/144338, WO 2018/107054, WO 2017/222453, WO 2019/068880, WO 2011/094669, U.S. Pat. Nos. 7,709,198, 8,604,182, 8,951,726, 9,783,841, 10,041,949, WO 2016/057552, WO 2017/147483, WO 2018/022809, WO 2016/166128, WO 2017/027367, WO 2017/027368, WO 2018/136856, WO 2019/075091, U.S. Pat. No. 10,059,990, WO 2018/057999, WO 2015/161173, Gupta et al., Nature Biotechnol. 36:1197-1202, 2018, and U.S. Patent Application Publication No. US20210062272A1 and can be used herein in any combination. Spatial analysis of analytes is further described in U.S. Pat. No. 11,501,440, U.S. Patent Application Publication No. US20210150707A1 entitled “SYSTEMS AND METHODS FOR TISSUE CLASSIFICATION,” U.S. Pat. No. 11,514,575, and U.S. Patent Application Publication No. US20210155982A1 entitled “Spatial Analysis of Analytes,” each of which is hereby incorporated herein by reference in its entirety.

Furthermore, high-resolution spatial mapping of analytes to their specific location within a region or subregion can reveal spatial expression patterns of analytes, provide relational data, and further implicate analyte network interactions relating to disease or other morphologies or phenotypes of interest, resulting in a holistic understanding of cells in their morphological context. See, e.g., 10×, 2019, “Spatially-Resolved Transcriptomics,” 10×, 2019, “Inside Visium Spatial Technology,” and 10×, 2019, “Visium Spatial Gene Expression Solution,” each of which is hereby incorporated herein by reference in its entirety.

### DEFINITIONS

Specific terminology is used throughout this disclosure to explain various aspects of the apparatus, systems, methods, and compositions that are described. This sub-section includes explanations of certain terms that appear in later sections of the disclosure. To the extent that the descriptions in this section are in apparent conflict with usage in other sections of this disclosure, the definitions in this section will control.

### (A) General Definitions

### Analytes

As used herein, the term “analyte” refers to any biological substance, structure, moiety, or component to be analyzed. The term “target” is similarly used herein to refer to an analyte of interest. In some embodiments, the apparatus, systems, methods, and compositions described in this disclosure can be used to detect and analyze a wide variety of different analytes.

Analytes can be broadly classified into one of two groups: nucleic acid analytes, and non-nucleic acid analytes. Examples of non-nucleic acid analytes include, but are not limited to, lipids, carbohydrates, peptides, proteins, glycoproteins (N-linked or O-linked), lipoproteins, phosphoproteins, specific phosphorylated or acetylated variants of proteins, amidation variants of proteins, hydroxylation variants of proteins, methylation variants of proteins, ubiquitylation variants of proteins, sulfation variants of proteins, viral proteins (e.g., viral capsid, viral envelope, viral coat, viral accessory, viral glycoproteins, viral spike, etc.), extracellular and intracellular proteins, antibodies, and antigen binding fragments. In some embodiments, the analyte is an organelle (e.g., nuclei or mitochondria). In some embodiments, the analyte(s) can be localized to subcellular location(s), including, for example, organelles, e.g., mitochondria, Golgi apparatus, endoplasmic reticulum, chloroplasts, endocytic vesicles, exocytic vesicles, vacuoles, lysosomes, etc. In some embodiments, analyte(s) can be peptides or proteins, including without limitation antibodies and enzymes. Additional examples of analytes can be found in Section (I) (c) of WO 2020/176788 and/or U.S. Patent Application Publication No. 2020/0277663. In some embodiments, an analyte can be detected indirectly, such as through detection of an intermediate agent, for example, a connected probe (e.g., a ligation product) or an analyte capture agent (e.g., an oligonucleotide-conjugated antibody), such as those described herein. In some embodiments, analytes can include one or more intermediate agents, e.g., connected probes or analyte capture agents that bind to nucleic acid, protein, or peptide analytes in a sample.

Cell surface features corresponding to analytes can include, but are not limited to, a receptor, an antigen, a surface protein, a transmembrane protein, a cluster of differentiation protein, a protein channel, a protein pump, a carrier protein, a phospholipid, a glycoprotein, a glycolipid, a cell-cell interaction protein complex, an antigen-presenting complex, a major histocompatibility complex, an engineered T-cell receptor, a T-cell receptor, a B-cell receptor, a chimeric antigen receptor, an extracellular matrix protein, a posttranslational modification (e.g., phosphorylation, glycosylation, ubiquitination, nitrosylation, methylation, acetylation or lipidation) state of a cell surface protein, a gap junction, and an adherens junction.

Analytes can be derived from a specific type of cell and/or a specific sub-cellular region. For example, analytes can be derived from cytosol, from cell nuclei, from mitochondria, from microsomes, and more generally, from any other compartment, organelle, or portion of a cell. Permeabilizing agents that specifically target certain cell compartments and organelles can be used to selectively release analytes from cells for analysis.

Examples of nucleic acid analytes include DNA analytes such as genomic DNA, methylated DNA, specific methylated DNA sequences, fragmented DNA, mitochondrial DNA, in situ synthesized PCR products, and RNA/DNA hybrids.

Examples of nucleic acid analytes also include RNA analytes such as various types of coding and non-coding RNA. Examples of the different types of RNA analytes include messenger RNA (mRNA), ribosomal RNA (rRNA), transfer RNA (tRNA), microRNA (miRNA), and viral RNA. The RNA can be a transcript (e.g., present in a tissue section). The RNA can be small (e.g., less than 200 nucleic acid bases in length) or large (e.g., RNA greater than 200 nucleic acid bases in length). Small RNAs mainly include 5.8S ribosomal RNA (rRNA), 5S rRNA, transfer RNA (tRNA), microRNA (miRNA), small interfering RNA (siRNA), small nucleolar RNA (snoRNAs), Piwi-interacting RNA (piRNA), tRNA-derived small RNA (tsRNA), and small rDNA-derived RNA (srRNA). The RNA can be double-stranded RNA or single-stranded RNA. The RNA can be circular RNA. The RNA can be a bacterial IRNA (e.g., 16s rRNA or 23s rRNA).

Additional examples of analytes include mRNA and cell surface features (e.g., using the labelling agents described herein), mRNA and intracellular proteins (e.g., transcription factors), mRNA and cell methylation status, mRNA and accessible chromatin (e.g., ATAC-seq, DNase-seq, and/or MNase-seq), mRNA and metabolites (e.g., using the labelling agents described herein), a barcoded labelling agent (e.g., the oligonucleotide tagged antibodies described herein) and a V(D)J sequence of an immune cell receptor (e.g., T-cell receptor), mRNA and a perturbation agent (e.g., a CRISPR crRNA/sgRNA, TALEN, zinc finger nuclease, and/or antisense oligonucleotide as described herein). In some embodiments, a perturbation agent is a small molecule, an antibody, a drug, an aptamer, a miRNA, a physical environmental (e.g., temperature change), or any other known perturbation agents.

Analytes can include a nucleic acid molecule with a nucleic acid sequence encoding at least a portion of a V(D)J sequence of an immune cell receptor (e.g., a TCR or BCR). In some embodiments, the nucleic acid molecule is cDNA first generated from reverse transcription of the corresponding mRNA, using a poly(T) containing primer. The generated cDNA can then be barcoded using a capture probe, featuring a barcode sequence (and optionally, a UMI sequence) that hybridizes with at least a portion of the generated cDNA. In some embodiments, a template switching oligonucleotide hybridizes to a poly(C) tail added to a 3′ end of the cDNA by a reverse transcriptase enzyme. The original mRNA template and template switching oligonucleotide can then be denatured from the cDNA and the barcoded capture probe can then hybridize with the cDNA and a complement of the cDNA generated. Additional methods and compositions suitable for barcoding cDNA generated from mRNA transcripts including those encoding V(D)J regions of an immune cell receptor and/or barcoding methods and composition including a template switch oligonucleotide are described in PCT Patent Application PCT/US2017/057269, filed Oct. 18, 2017, and U.S. patent application Ser. No. 15/825,740, filed Nov. 29, 2017, both of which are incorporated herein by reference in their entireties. V(D)J analysis can also be completed with the use of one or more labelling agents that bind to particular surface features of immune cells and associated with barcode sequences. The one or more labelling agents can include an MHC or MHC multimer.

As described above, the analyte can include a nucleic acid capable of functioning as a component of a gene editing reaction, such as, for example, clustered regularly interspaced short palindromic repeats (CRISPR)-based gene editing. Accordingly, the capture probe can include a nucleic acid sequence that is complementary to the analyte (e.g., a sequence that can hybridize to the CRISPR RNA (crRNA), single guide RNA (sgRNA), or an adapter sequence engineered into a crRNA or sgRNA).

In certain embodiments, an analyte is extracted from a live cell. Processing conditions can be adjusted to ensure that a biological sample remains live during analysis, and analytes are extracted from (or released from) live cells of the sample. Live cell-derived analytes can be obtained only once from the sample or can be obtained at intervals from a sample that continues to remain in viable condition.

In general, the systems, apparatus, methods, and compositions can be used to analyze any number of analytes. For example, the number of analytes that are analyzed can be at least about 2, at least about 3, at least about 4, at least about 5, at least about 6, at least about 7, at least about 8, at least about 9, at least about 10, at least about 11, at least about 12, at least about 13, at least about 14, at least about 15, at least about 20, at least about 25, at least about 30, at least about 40, at least about 50, at least about 100, at least about 1,000, at least about 10,000, at least about 100,000 or more different analytes present in a region of the sample or within an individual capture spot of the substrate. Methods for performing multiplexed assays to analyze two or more different analytes will be discussed in a subsequent section of this disclosure.

In some embodiments, more than one analyte type (e.g., nucleic acids and proteins) from a biological sample can be detected (e.g., simultaneously or sequentially) using any appropriate multiplexing technique, such as those described in Section (IV) of WO 2020/176788 and/or U.S. Patent Application Publication No. 2020/0277663.

In some embodiments, detection of one or more analytes (e.g., protein analytes) can be performed using one or more analyte capture agents. As used herein, an “analyte capture agent” refers to an agent that interacts with an analyte (e.g., an analyte in a biological sample) and with a capture probe (e.g., a capture probe attached to a substrate or a feature) to identify the analyte. In some embodiments, the analyte capture agent includes: (i) an analyte binding moiety (e.g., that binds to an analyte), for example, an antibody or antigen-binding fragment thereof; (ii) analyte binding moiety barcode; and (iii) a capture handle sequence. As used herein, the term “analyte binding moiety barcode” refers to a barcode that is associated with or otherwise identifies the analyte binding moiety. As used herein, the term “analyte capture sequence” or “capture handle sequence” refers to a region or moiety configured to hybridize to, bind to, couple to, or otherwise interact with a capture domain of a capture probe. In some embodiments, a capture handle sequence is complementary to a capture domain of a capture probe. In some cases, an analyte binding moiety barcode (or portion thereof) may be able to be removed (e.g., cleaved) from the analyte capture agent.

Additional examples of analytes suitable for use in the present disclosure are described in U.S. Patent Application Publication Nos. US20210158522A1; US20210150707A1; US20210097684A1 and US20210155982A1, each of which is hereby incorporated herein by reference in its entirety.

### Barcodes

As used herein, the term “barcode” refers to a label, or identifier, that conveys or is capable of conveying information (e.g., information about an analyte in a sample, a bead, and/or a capture probe). A barcode can be part of an analyte, or independent of an analyte. A barcode can be attached to an analyte. A particular barcode can be unique relative to other barcodes. Barcodes suitable for use in the present disclosure are further described in U.S. Patent Application Publication Nos. US20210158522A1, US20210150707A1, US20210097684A1, and US20210155982A1, each of which is hereby incorporated herein by reference in its entirety.

### Biological Samples

As used herein, the term “sample” or “biological sample” refers to any material obtained from a subject for analysis using any of a variety of techniques including, but not limited to, biopsy, surgery, and laser capture microscopy (LCM), and generally includes cells and/or other biological material from the subject. In addition to the subjects described above, a biological sample can also be obtained from non-mammalian organisms (e.g., plants, insects, arachnids, nematodes, fungi, amphibians, and fish. A biological sample can be obtained from a prokaryote such as a bacterium, e.g., Escherichia coli, Staphylococci or Mycoplasma pneumoniae; archaea; a virus such as Hepatitis C virus or human immunodeficiency virus; or a viroid. A biological sample can also be obtained from a eukaryote, such as a patient derived organoid (PDO) or patient derived xenograft (PDX). The biological sample can include organoids, a miniaturized and simplified version of an organ produced in vitro in three dimensions that shows realistic micro-anatomy. Organoids can be generated from one or more cells from a tissue, embryonic stem cells, and/or induced pluripotent stem cells, which can self-organize in three-dimensional culture owing to their self-renewal and differentiation capacities. In some embodiments, an organoid is a cerebral organoid, an intestinal organoid, a stomach organoid, a lingual organoid, a thyroid organoid, a thymic organoid, a testicular organoid, a hepatic organoid, a pancreatic organoid, an epithelial organoid, a lung organoid, a kidney organoid, a gastruloid, a cardiac organoid, or a retinal organoid. Subjects from which biological samples can be obtained can be healthy or asymptomatic individuals, individuals that have or are suspected of having a disease (e.g., cancer) or a pre-disposition to a disease, and/or individuals that are in need of therapy or suspected of needing therapy.

The biological sample can include any number of macromolecules, for example, cellular macromolecules and organelles (e.g., mitochondria and nuclei). The biological sample can be a nucleic acid sample and/or protein sample. The biological sample can be a nucleic acid sample and/or protein sample. The biological sample can be a carbohydrate sample or a lipid sample. The biological sample can be obtained as a tissue sample, such as a tissue section, biopsy, a core biopsy, needle aspirate, or fine needle aspirate. The sample can be a fluid sample, such as a blood sample, urine sample, or saliva sample. The sample can be a skin sample, a colon sample, a cheek swab, a histology sample, a histopathology sample, a plasma or serum sample, a tumor sample, living cells, cultured cells, a clinical sample such as, for example, whole blood or blood-derived products, blood cells, or cultured tissues or cells, including cell suspensions and/or disaggregated cells.

Cell-free biological samples can include extracellular polynucleotides. Extracellular polynucleotides can be isolated from a bodily sample, e.g., blood, plasma, serum, urine, saliva, mucosal excretions, sputum, stool, and tears.

Biological samples can be derived from a homogeneous culture or population of the subjects or organisms mentioned herein or alternatively from a collection of several different organisms, for example, in a community or ecosystem.

Biological samples can include one or more diseased cells. A diseased cell can have altered metabolic properties, gene expression, protein expression, and/or morphologic features. Examples of diseases include inflammatory disorders, metabolic disorders, nervous system disorders, and cancer. Cancer cells can be derived from solid tumors, hematological malignancies, cell lines, or obtained as circulating tumor cells.

Biological samples can also include fetal cells. For example, a procedure such as amniocentesis can be performed to obtain a fetal cell sample from maternal circulation. Sequencing of fetal cells can be used to identify any of a number of genetic disorders, including, e.g., aneuploidy such as Down's syndrome, Edwards syndrome, and Patau syndrome. Further, cell surface features of fetal cells can be used to identify any of a number of disorders or diseases.

Biological samples can also include immune cells. Sequence analysis of the immune repertoire of such cells, including genomic, proteomic, and cell surface features, can provide a wealth of information to facilitate an understanding the status and function of the immune system. By way of example, determining the status (e.g., negative or positive) of minimal residue disease (MRD) in a multiple myeloma (MM) patient following autologous stem cell transplantation is considered a predictor of MRD in the MM patient (see, e.g., U.S. Patent Publication No. 2018/0156784, the entire contents of which are incorporated herein by reference).

Examples of immune cells in a biological sample include, but are not limited to, B cells, T cells (e.g., cytotoxic T cells, natural killer T cells, regulatory T cells, and T helper cells), natural killer cells, cytokine induced killer (CIK) cells, myeloid cells, such as granulocytes (basophil granulocytes, eosinophil granulocytes, neutrophil granulocytes/hyper-segmented neutrophils), monocytes/macrophages, mast cells, thrombocytes/megakaryocytes, and dendritic cells.

As discussed above, a biological sample can include a single analyte of interest, or more than one analyte of interest. Methods for performing multiplexed assays to analyze two or more different analytes in a single biological sample will be discussed in a subsequent section of this disclosure.

A variety of steps can be performed to prepare a biological sample for analysis. Except where indicated otherwise, the preparative steps for biological samples can generally be combined in any manner to appropriately prepare a particular sample for analysis.

For instance, in some embodiments, the biological sample is a tissue section. In some embodiments, the biological sample is prepared using tissue sectioning. A biological sample can be harvested from a subject (e.g., via surgical biopsy, whole subject sectioning, grown in vitro on a growth substrate or culture dish as a population of cells, or prepared for analysis as a tissue slice or tissue section). Grown samples may be sufficiently thin for analysis without further processing steps. Alternatively, grown samples, and samples obtained via biopsy or sectioning, can be prepared as thin tissue sections using a mechanical cutting apparatus such as a vibrating blade microtome. As another alternative, in some embodiments, a thin tissue section can be prepared by applying a touch imprint of a biological sample to a suitable substrate material. The thickness of the tissue section can be a fraction of (e.g., less than 0.9, 0.8, 0.7, 0.6, 0.5, 0.4, 0.3, 0.2, or 0.1) the maximum cross-sectional dimension of a cell. However, tissue sections having a thickness that is larger than the maximum cross-section cell dimension can also be used. For example, cryostat sections can be used, which can be, e.g., 10-20 micrometers thick.

More generally, the thickness of a tissue section typically depends on the method used to prepare the section and the physical characteristics of the tissue, and therefore sections having a wide variety of different thicknesses can be prepared and used. For example, the thickness of the tissue section can be at least 0.1, 0.2, 0.3, 0.4, 0.5, 0.7, 1.0, 1.5, 2, 3, 4, 5, 6, 7, 8, 9, 10, 11, 12, 13, 14, 15, 20, 30, 40, or 50 micrometers. Thicker sections can also be used if desired or convenient, e.g., at least 70, 80, 90, or 100 micrometers or more. Typically, the thickness of a tissue section is between 1-100 micrometers, 1-50 micrometers, 1-30 micrometers, 1-25 micrometers, 1-20 micrometers, 1-15 micrometers, 1-10 micrometers, 2-8 micrometers, 3-7 micrometers, or 4-6 micrometers, but as mentioned above, sections with thicknesses larger or smaller than these ranges can also be analysed.

In some embodiments, a tissue section is a similar size and shape to a substrate (e.g., the first substrate and/or the second substrate). In some embodiments, a tissue section is a different size and shape from a substrate. In some embodiments, a tissue section is on all or a portion of the substrate. In some embodiments a tissue section has dimensions roughly comparable to the substrate, such that a large proportion of the substrate is in contact with the tissue section. In some embodiments, several biological samples from a subject are concurrently analyzed. For instance, in some embodiments several different sections of a tissue are concurrently analyzed. In some embodiments, 1, 2, 3, 4, 5, 6, 7, 8, 9, 10, 11, 12, 13, 14, 15, 16, 17, 18, 19, or 20 different biological samples from a subject are concurrently analyzed. For example, in some embodiments, 1, 2, 3, 4, 5, 6, 7, 8, 9, 10, 11, 12, 13, 14, 15, 16, 17, 18, 19, or 20 different tissue sections from a single biological sample from a single subject are concurrently analyzed. In some embodiments, one or more images are acquired of each such tissue section.

In some embodiments, a tissue section on a substrate is a single uniform section. In some embodiments, multiple tissue sections are on a substrate. In some such embodiments, a single capture area can contain multiple tissue sections, where each tissue section is obtained from either the same biological sample and/or subject or from different biological samples and/or subjects. In some embodiments, a tissue section is a single tissue section that comprises one or more regions where no cells are present (e.g., holes, tears, or gaps in the tissue). Thus, in some embodiments, such as the above, an image of a tissue section on a substrate can contain regions where tissue is present and regions where tissue is not present.

Additional examples of tissue samples are shown in Table 1 and catalogued, for example, in 10×, 2019, “Visium Spatial Gene Expression Solution,” and in U.S. Patent Application Publication Nos. US20210158522A1, US20210150707A1; US20210097684A1, and US20210155982A1, each of which is hereby incorporated herein by reference in its entirety.

TABLE 1
Examples of tissue samples

| Organism | Tissue | Healthy/Diseased |
|---|---|---|
| Human | Brain | Cerebrum Glioblastoma Multiforme |
| Human | Breast | Healthy |
| Human | Breast | Invasive Ductal Carcinoma |
| Human | Breast | Invasive Lobular Carcinoma |
| Human | Heart | Healthy |
| Human | Kidney | Healthy |
| Human | Kidney | Nephritis |
| Human | Large Intestine | Colorectal Cancer |
| Human | Lung | Papillary Carcinoma |
| Human | Lymph Node | Healthy |
| Human | Lymph Node | Inflamed |
| Human | Ovaries | Tumor |
| Human | Spleen | Inflamed |
| Mouse | Brain | Healthy |
| Mouse | Eyes | Healthy |
| Mouse | Heart | Healthy |
| Mouse | Kidney | Healthy |
| Mouse | Large Intestine | Healthy |
| Mouse | Liver | Healthy |
| Mouse | Lungs | Healthy |
| Mouse | Ovary | Healthy |
| Mouse | Quadriceps | Healthy |
| Mouse | Small Intestine | Healthy |
| Mouse | Spleen | Healthy |
| Mouse | Stomach | Healthy |
| Mouse | Testes | Healthy |
| Mouse | Thyroid | Healthy |
| Mouse | Tongue | Healthy |
| Rat | Brain | Healthy |
| Rat | Heart | Healthy |
| Rat | Kidney | Healthy |
| Mouse | Tongue | Healthy |
| Rat | Brain | Healthy |
| Rat | Heart | Healthy |
| Rat | Kidney | Healthy |

Multiple sections can also be obtained from a single biological sample. For example, multiple tissue sections can be obtained from a surgical biopsy sample by performing serial sectioning of the biopsy sample using a sectioning blade. Spatial information among the serial sections can be preserved in this manner, and the sections can be analysed successively to obtain three-dimensional information about the biological sample.

In some embodiments, a biological sample is prepared using one or more steps including, but not limited to, freezing, fixation, embedding, formalin fixation and paraffin embedding, hydrogel embedding, biological sample transfer, isometric expansion, cell disaggregation, cell suspension, cell adhesion, permeabilization, lysis, protease digestion, selective permeabilization, selective lysis, selective enrichment, enzyme treatment, library preparation, and/or sequencing pre-processing. Methods for biological sample preparation that are contemplated in the present disclosure are described in further detail in U.S. Pat. No. 11,501,440; U.S. Patent Publication No. US20210150707A1; U.S. Pat. No. 11,514,575, and U.S. Patent Publication No. US20210155982A1, each of which is hereby incorporated herein by reference in its entirety.

In some embodiments, a biological sample is prepared by staining. To facilitate visualization, biological samples can be stained using a wide variety of stains and staining techniques. In some embodiments, for example, a sample can be stained using any number of biological stains, including but not limited to, acridine orange, Bismarck brown, carmine, Coomassie blue, cresyl violet, DAPI, eosin, ethidium bromide, acid fuchsine, hematoxylin, Hoechst stains, iodine, methyl green, methylene blue, neutral red, Nile blue, Nile red, osmium tetroxide, propidium iodide, rhodamine, safranin, or a combination thereof.

The sample can be stained using known staining techniques, including Can-Grunwald, Giemsa, hematoxylin and eosin (H&E), Jenner's, Leishman, Masson's trichrome, Papanicolaou, Romanowsky, silver, Sudan, Wright's, and/or Periodic Acid Schiff (PAS) staining techniques. PAS staining is typically performed after formalin or acetone fixation.

In some embodiments, the sample is stained using a detectable label (e.g., radioisotopes, fluorophores, chemiluminescent compounds, bioluminescent compounds, and dyes). In some embodiments, a biological sample is stained using only one type of stain or one technique. In some embodiments, staining includes biological staining techniques such as H&E staining. In some embodiments, staining includes identifying analytes using fluorescently-labeled antibodies. In some embodiments, a biological sample is stained using two or more different types of stains, or two or more different staining techniques. For example, a biological sample can be prepared by staining and imaging using one technique (e.g., H&E staining and bright-field imaging), followed by staining and imaging using another technique (e.g., IHC/IF staining and fluorescence microscopy) for the same biological sample.

In some embodiments, biological samples can be destained. Methods of destaining or discoloring a biological sample are known in the art, and generally depend on the nature of the stain(s) applied to the sample. For example, H&E staining can be destained by washing the sample in HCl, or any other low pH acid (e.g., selenic acid, sulfuric acid, hydroiodic acid, benzoic acid, carbonic acid, malic acid, phosphoric acid, oxalic acid, succinic acid, salicylic acid, tartaric acid, sulfurous acid, trichloroacetic acid, hydrobromic acid, hydrochloric acid, nitric acid, orthophosphoric acid, arsenic acid, selenous acid, chromic acid, citric acid, hydrofluoric acid, nitrous acid, isocyanic acid, formic acid, hydrogen selenide, molybdic acid, lactic acid, acetic acid, carbonic acid, hydrogen sulfide, or combinations thereof). In some embodiments, destaining can include 1, 2, 3, 4, 5, or more washes in a low pH acid (e.g., HCl). In some embodiments, destaining can include adding HCl to a downstream solution (e.g., permeabilization solution). In some embodiments, destaining can include dissolving an enzyme used in the disclosed methods (e.g., pepsin) in a low pH acid (e.g., HCl) solution. In some embodiments, after destaining hematoxylin with a low pH acid, other reagents can be added to the destaining solution to raise the pH for use in other applications. For example, SDS can be added to a low pH acid destaining solution in order to raise the pH as compared to the low pH acid destaining solution alone. As another example, in some embodiments, one or more immunofluorescence stains are applied to the sample via antibody coupling. Such stains can be removed using techniques such as cleavage of disulfide linkages via treatment with a reducing agent and detergent washing, chaotropic salt treatment, treatment with antigen retrieval solution, and treatment with an acidic glycine buffer. Methods for multiplexed staining and destaining are described, for example, in Bolognesi et al., 2017, J. Histochem. Cytochem. 65(8): 431-444, Lin et al., 2015, Nat Commun. 6:8390, Pirici et al., 2009, J. Histochem. Cytochem. 57:567-75, and Glass et al., 2009, J. Histochem. Cytochem. 57:899-905, the entire contents of each of which are incorporated herein by reference.

In some embodiments, the biological sample can be attached to a substrate (e.g., a slide and/or a chip). Examples of substrates suitable for this purpose are described in detail elsewhere herein (see, for example, Definitions: “Substrates,” below). Attachment of the biological sample can be irreversible or reversible, depending upon the nature of the sample and subsequent steps in the analytical method.

In certain embodiments, the sample can be attached to the substrate reversibly by applying a suitable polymer coating to the substrate and contacting the sample to the polymer coating. The sample can then be detached from the substrate using an organic solvent that at least partially dissolves the polymer coating. Hydrogels are examples of polymers that are suitable for this purpose. More generally, in some embodiments, the substrate can be coated or functionalized with one or more substances to facilitate attachment of the sample to the substrate. Suitable substances that can be used to coat or functionalize the substrate include, but are not limited to, lectins, poly-lysine, antibodies, and polysaccharides.

Biological samples contemplated for use in the present disclosure are further described in U.S. Pat. No. 11,501,440; U.S. Patent Publication No. US20210150707A1; U.S. Pat. No. 11,514,575, and U.S. Patent Publication No. US20210155982A1, each of which is hereby incorporated herein by reference in its entirety, each of which is hereby incorporated herein by reference in its entirety.

### Capture Probes

A “capture probe,” also interchangeably referred to herein as a “probe,” refers to any molecule capable of capturing (directly or indirectly) and/or labelling an analyte (e.g., an analyte of interest) in a biological sample. In some embodiments, the capture probe is a nucleic acid or a polypeptide. In some embodiments, the capture probe is a conjugate (e.g., an oligonucleotide-antibody conjugate). In some embodiments, the capture probe includes a barcode (e.g., a spatial barcode and/or a unique molecular identifier (UMI)) and a capture domain.

In some embodiments a capture probe is optionally coupled to a capture spot (feature) on an arrayed substrate, for example by a cleavage domain, such as a disulfide linker.

The capture probe can include functional sequences that are useful for subsequent processing, such as a first functional sequence, which can include a sequencer specific flow cell attachment sequence, e.g., a P5 sequence, as well as a second functional sequence, which can include sequencing primer sequences, e.g., an R1 primer binding site, an R2 primer binding site. In some embodiments, first functional sequence is a P7 sequence and second functional sequence is a R2 primer binding site.

A spatial barcode can be included within the capture probe for use in determining the relative location of the target analyte in the biological sample. Additional functional sequences can be selected for compatibility with a variety of different sequencing systems, e.g., 454 Sequencing, Ion Torrent Proton or PGM, Illumina sequencing instruments, PacBio, Oxford Nanopore, etc., and the requirements thereof. In some embodiments, functional sequences can be selected for compatibility with non-commercialized sequencing systems. Examples of such sequencing systems and techniques, for which suitable functional sequences can be used, include (but are not limited to) Ion Torrent Proton or PGM sequencing, Illumina sequencing, PacBio SMRT sequencing, and Oxford Nanopore sequencing. Further, in some embodiments, functional sequences can be selected for compatibility with other sequencing systems, including non-commercialized sequencing systems.

In some embodiments, the spatial barcode, first functional sequence (e.g., flow cell attachment sequence) and second functional sequence (e.g., sequencing primer sequences) can be common to all of the probes attached to a given capture spot. The capture probe also includes a capture domain for capturing a target analyte.

Other aspects of capture probes contemplated for use in the present disclosure are known in the art. For instance, example suitable cleavage domains are described in further detail in PCT publication No. 202020176788A1 the entire contents of which is incorporated herein by reference. Example suitable functional domains are described in further detail in U.S. Patent Application Publication No. US20210062272A1 as well as PCT publication No. 202020176788A1, entitled “Profiling of biological analytes with spatially barcoded oligonucleotide arrays,” each of which is hereby incorporated herein by reference. Example suitable spatial barcodes and unique molecular identifiers are described in further detail in U.S. Patent Application Publication No. US20210062272A1 and PCT publication No. 202020176788A1 each of which is hereby incorporated herein by reference.

Capture probes contemplated for use in the present disclosure are further described in U.S. Pat. No. 11,501,440; U.S. Patent Publication No. US20210150707A1; U.S. Pat. No. 11,514,575, and U.S. Patent Publication No. US20210155982A1, each of which is hereby incorporated herein by reference in its entirety, each of which is hereby incorporated herein by reference in its entirety.

### Capture Spots

As used interchangeably herein, the terms “capture spot,” “capture feature,” “feature”, “capture area,” or “capture probe plurality” refer to an entity that acts as a support or repository for various molecular entities used in sample analysis. Examples of capture spots include, but are not limited to, a bead, a spot of any two- or three-dimensional geometry (e.g., an ink jet spot, a masked spot, a square on a grid), a well, and a hydrogel pad. In some embodiments, a capture spot is an area on a substrate at which capture probes labelled with spatial barcodes are located. Specific non-limiting embodiments of capture spots and substrates are further described below in the present disclosure.

In some embodiments, capture spots are directly or indirectly attached or fixed to a substrate (e.g., of a chip or a slide). In some embodiments, the capture spots are not directly or indirectly attached or fixed to a substrate, but instead, for example, are disposed within an enclosed or partially enclosed three dimensional space (e.g., wells or divots). In some embodiments, some or all capture spots in an array include a capture probe.

In some embodiments, a capture spot includes different types of capture probes attached to the capture spot. For example, the capture spot can include a first type of capture probe with a capture domain designed to bind to one type of analyte, and a second type of capture probe with a capture domain designed to bind to a second type of analyte. In general, capture spots can include one or more (e.g., two or more, three or more, four or more, five or more, six or more, eight or more, ten or more, 12 or more, 15 or more, 20 or more, 30 or more, 50 or more) different types of capture probes attached to a single capture spot.

In some embodiments, a capture spot on the array includes a bead. In some embodiments, two or more beads are dispersed onto a substrate to create an array, where each bead is a capture spot on the array. Beads can optionally be dispersed into wells on a substrate, e.g., such that only a single bead is accommodated per well.

Further details and non-limiting embodiments relating to capture spots (features), including but not limited to beads, bead arrays, bead properties (e.g., structure, materials, construction, cross-linking, degradation, reagents, and/or optical properties), and for covalently and non-covalently bonding beads to substrates are described in U.S. Patent Application Publication No. US20210062272A1, U.S. Patent Application Publication No. 20110059865A1, U.S. Provisional Application No. 62/839,346, U.S. Pat. No. 9,012,022, PCT publication No. 202020176788A1, entitled “Profiling of biological analytes with spatially barcoded oligonucleotide arrays,” U.S. Pat. No. 11,501,440, U.S. Patent Application Publication No. US20210150707A1; U.S. Pat. No. 11,514,575; and U.S. Patent Application Publication No. US20210155982A1, each of which is hereby incorporated herein by reference in its entirety.

### Capture Spot Arrays

In some embodiments, capture spots are collectively positioned on a substrate. As used herein, the term “capture spot array” or “array” refers to a specific arrangement of a plurality of capture spots (also termed “features”) that is either irregular or forms a regular pattern. Individual capture spots in the array differ from one another based on their relative spatial locations. In general, at least two of the plurality of capture spots in the array include a distinct capture probe (e.g., any of the examples of capture probes described herein).

Arrays can be used to measure large numbers of analytes simultaneously. In some embodiments, oligonucleotides are used, at least in part, to create an array. For example, one or more copies of a single species of oligonucleotide (e.g., capture probe) can correspond to or be directly or indirectly attached to a given capture spot in the array. In some embodiments, a given capture spot in the array includes two or more species of oligonucleotides (e.g., capture probes). In some embodiments, the two or more species of oligonucleotides (e.g., capture probes) attached directly or indirectly to a given capture spot on the array include a common (e.g., identical) spatial barcode.

In some embodiments, a substrate and/or an array (e.g., two-dimensional array) comprises a plurality of capture spots. In some embodiments, a substrate and/or an array includes between 4000 and 10,000 capture spots, or any range within 4000 to 6000 capture spots. For example, a substrate and/or an array includes between 4,000 to 4,400 capture spots, 4,000 to 4,800 capture spots, 4,000 to 5,200 capture spots, 4,000 to 5,600 capture spots, 5,600 to 6,000 capture spots, 5,200 to 6,000 capture spots, 4,800 to 6,000 capture spots, or 4,400 to 6,000 capture spots. In some embodiments, the substrate and/or array includes between 4,100 and 5,900 capture spots, between 4,200 and 5,800 capture spots, between 4,300 and 5,700 capture spots, between 4,400 and 5,600 capture spots, between 4,500 and 5,500 capture spots, between 4,600 and 5,400 capture spots, between 4,700 and 5,300 capture spots, between 4,800 and 5,200 capture spots, between 4,900 and 5,100 capture spots, or any range within the disclosed sub-ranges. For example, the substrate and/or array can include about 4,000 capture spots, about 4,200 capture spots, about 4,400 capture spots, about 4,800 capture spots, about 5,000 capture spots, about 5,200 capture spots, about 5,400 capture spots, about 5,600 capture spots, or about 6,000 capture spots. In some embodiments, the substrate and/or array comprises at least 4,000 capture spots. In some embodiments, the substrate and/or array includes approximately 5,000 capture spots.

Arrays suitable for use in the present disclosure are further described in PCT publication 202020176788A1, entitled “Profiling of biological analytes with spatially barcoded oligonucleotide arrays”, as well as U.S. Pat. No. 11,501,440; U.S. Patent Publication No. US20210150707A1; U.S. Pat. No. 11,514,575, and U.S. Patent Publication No. US20210155982A1, each of which is hereby incorporated herein by reference in its entirety, each of which is hereby incorporated herein by reference in its entirety.

### Contact

As used herein, the terms “contact,” “contacted,” and/or “contacting” of a biological sample with a substrate comprising capture spots refers to any contact (e.g., direct or indirect) such that capture probes can interact (e.g., capture) with analytes from the biological sample. For example, the substrate may be near or adjacent to the biological sample without direct physical contact, yet capable of capturing analytes from the biological sample. In some embodiments the biological sample is in direct physical contact with the substrate. In some embodiments, the biological sample is in indirect physical contact with the substrate. For example, a liquid layer may be between the biological sample and the substrate. In some embodiments, the analytes diffuse through the liquid layer. In some embodiments the capture probes diffuse through the liquid layer. In some embodiments, reagents may be delivered via the liquid layer between the biological sample and the substrate. In some embodiments, indirect physical contact may be the presence of a second substrate (e.g., a hydrogel, a film, a porous membrane) between the biological sample and the first substrate comprising capture spots with capture probes. In some embodiments, reagents are delivered by the second substrate to the biological sample.

In some embodiments, a cell immobilization agent can be used to contact a biological sample with a substrate (e.g., by immobilizing non-aggregated or disaggregated sample on a spatially-barcoded array prior to analyte capture). A “cell immobilization agent” as used herein can refer to an agent (e.g., an antibody), attached to a substrate, which can bind to a cell surface marker. Non-limiting examples of a cell surface marker include CD45, CD3, CD4, CD8, CD56, CD19, CD20, CD11c, CD14, CD33, CD66b, CD34, CD41, CD61, CD235a, CD146, and epithelial cellular adhesion molecule (EpCAM). A cell immobilization agent can include any probe or component that can bind to (e.g., immobilize) a cell or tissue when on a substrate. A cell immobilization agent attached to the surface of a substrate can be used to bind a cell that has a cell surface maker. The cell surface marker can be a ubiquitous cell surface marker, where the purpose of the cell immobilization agent is to capture a high percentage of cells within the sample. The cell surface marker can be a specific, or more rarely expressed, cell surface marker, where the purpose of the cell immobilization agent is to capture a specific cell population expressing the target cell surface marker. Accordingly, a cell immobilization agent can be used to selectively capture a cell expressing the target cell surface marker from a population of cells that do not have the same cell surface marker.

Generally, analytes can be captured when contacting a biological sample with, e.g., a substrate comprising capture probes (e.g., substrate with capture probes embedded, spotted, printed on the substrate or a substrate with capture spots (e.g., beads, wells) comprising capture probes). Capture can be performed using passive capture methods and/or active capture methods.

In some embodiments, capture of analytes is facilitated by treating the biological sample with permeabilization reagents. If a biological sample is not permeabilized sufficiently, the amount of analyte captured on the substrate can be too low to enable adequate analysis. Conversely, if the biological sample is too permeable, the analyte can diffuse away from its origin in the biological sample, such that the relative spatial relationship of the analytes within the biological sample is lost. Hence, a balance between permeabilizing the biological sample enough to obtain good signal intensity while still maintaining the spatial resolution of the analyte distribution in the biological sample is desired. Methods of preparing biological samples to facilitate capture are known in the art and can be modified depending on the biological sample and how the biological sample is prepared (e.g., fresh frozen, FFPE, etc.). Examples of analyte capture suitable for use in the present disclosure are further described in U.S. Pat. No. 11,501,440; U.S. Patent Publication No. US20210150707A1; U.S. Pat. No. 11,514,575, and U.S. Patent Publication No. US20210155982A1, each of which is hereby incorporated herein by reference in its entirety, each of which is hereby incorporated herein by reference in its entirety.

### Fiducials

As used interchangeably herein, the terms “fiducial,” “spatial fiducial,” “fiducial marker,” and “fiducial spot” generally refers to a point of reference or measurement scale. In some embodiments, imaging is performed using one or more fiducial markers, i.e., objects placed in the field of view of an imaging system that appear in the image produced. Fiducial markers can include, but are not limited to, detectable labels such as fluorescent, radioactive, chemiluminescent, calorimetric, and colorimetric labels. The use of fiducial markers to stabilize and orient biological samples is described, for example, in Carter et al., Applied Optics 46:421-427, 2007), the entire contents of which are incorporated herein by reference.

In some embodiments, a fiducial marker can be present on a substrate to provide orientation of the biological sample. In some embodiments, a microsphere can be coupled to a substrate to aid in orientation of the biological sample. In some examples, a microsphere coupled to a substrate can produce an optical signal (e.g., fluorescence). In another example, a microsphere can be attached to a portion (e.g., corner) of an array in a specific pattern or design (e.g., hexagonal design) to aid in orientation of a biological sample on an array of capture spots on the substrate. In some embodiments, a fiducial marker can be an immobilized molecule with which a detectable signal molecule can interact to generate a signal. For example, a marker nucleic acid can be linked or coupled to a chemical moiety capable of fluorescing when subjected to light of a specific wavelength (or range of wavelengths). Such a marker nucleic acid molecule can be contacted with an array before, contemporaneously with, or after the tissue sample is stained to visualize or image the tissue section. In some embodiments, it can be advantageous to use a marker that can be detected using the same conditions (e.g., imaging conditions) used to detect an analyte of interest.

In some embodiments, fiducial markers are included to facilitate the orientation of a tissue sample or an image thereof in relation to an immobilized capture probes on a substrate. Any number of methods for marking an array can be used such that a marker is detectable only when a tissue section is imaged. For instance, a molecule, e.g., a fluorescent molecule that generates a signal, can be immobilized directly or indirectly on the surface of a substrate. Markers can be provided on a substrate in a pattern (e.g., an edge, one or more rows, one or more lines, etc.).

In some embodiments, a fiducial marker can be stamped, attached, or synthesized on the substrate and contacted with a biological sample. Typically, an image of the sample and the fiducial marker is taken, and the position of the fiducial marker on the substrate can be confirmed by viewing the image.

In some examples, fiducial markers can surround the array. In some embodiments the fiducial markers allow for detection of, e.g., mirroring. In some embodiments, the fiducial markers may completely surround the array. In some embodiments, the fiducial markers may not completely surround the array. In some embodiments, the fiducial markers identify the corners of the array. In some embodiments, one or more fiducial markers identify the center of the array.

Example spatial fiducials suitable for use in the present disclosure are further described in U.S. Pat. No. 11,501,440; U.S. Patent Publication No. US20210150707A1; U.S. Pat. No. 11,514,575, and U.S. Patent Publication No. US20210155982A1, each of which is hereby incorporated herein by reference in its entirety, each of which is hereby incorporated herein by reference in its entirety.

### Imaging

In the context of a first image for a particular nucleic acid analyte, “imaging” refers to a count of unique molecular identifiers for a particular nucleic acid analyte at particular locations on a substrate onto which a biological sample (e.g., tissue or array of cells) is overlayed. In the context of a second image for a particular non-nucleic acid analyte, in some embodiments “imaging” refers to a count of unique molecular identifiers for a particular non-nucleic acid analyte at particular locations on a substrate onto which the biological sample (e.g., tissue or array of cells) is overlayed.

In alternative embodiments, as used herein, the term “imaging” refers, in the context of a second image for a non-nucleic acid analyte, to any method of obtaining an image, e.g., a microscope image. For example, images include bright-field images, which are transmission microscopy images where broad-spectrum, white light is placed on one side of the sample mounted on a substrate and the camera objective is placed on the other side and the sample itself filters the light in order to generate colors or grayscale intensity images.

In some embodiments, in addition to or instead of bright-field imaging, emission imaging, such as fluorescence imaging is used. In emission imaging approaches, the sample on the substrate is exposed to light of a specific narrow band (first wavelength band) of light and the light that is re-emitted from the sample at a slightly different wavelength (second wavelength band) is measured. This absorption and re-emission is due to the presence of a fluorophore that is sensitive to the excitation used and can be either a natural property of the sample or an agent the sample has been exposed to in preparation for the imaging. As an example, in an immunofluorescence experiment, an antibody that binds to a certain protein or class of proteins, and that is labeled with a certain fluorophore, is added to the sample. The locations on the sample that include the protein or class of proteins will then emit the second wavelength band. In some implementations, multiple antibodies with multiple fluorophores can be used to label multiple proteins in the sample. Each such fluorophore undergoes excitation with a different wavelength of light and further emits a different unique wavelength of light. In order to spatially resolve each of the different emitted wavelengths of light, the sample is subjected to the different wavelengths of light that will excite the multiple fluorophores on a serial basis and images for each of these light exposures is saved as an image thus generating a plurality of images. For instance, the image is subjected to a first wavelength that excites a first fluorophore to emit at a second wavelength and a first image of the sample is taken while the sample is being exposed to the first wavelength. The exposure of the sample to the first wavelength is discontinued and the sample is exposed to a third wavelength (different from the first wavelength) that excites a second fluorophore at a fourth wavelength (different from the second wavelength) and a second image of the sample is taken while the sample is being exposed to the third wavelength. Such a process is repeated for each different fluorophore in the multiple fluorophores (e.g., two or more fluorophores, three or more fluorophores, four or more fluorophores, five or more fluorophores). In this way, a series of images of the tissue, each depicting the spatial arrangement of some different parameter such as a particular protein or protein class, is obtained. In some embodiments, more than one fluorophore is imaged at the same time. In such an approach a combination of excitation wavelengths are used, each for one of the more than one fluorophores, and a single image is collected.

In some embodiments, each of the images collected through emission imaging is a grayscale image. To differentiate such grayscaled images, in some embodiments each of the images are assigned a color (shades of red, shades of blue, etc.). In some implementations, each image is then combined into one composite color image for viewing. This allows for the spatial analysis of analytes (e.g., spatial proteomics, spatial transcriptomics, etc.) in the sample. In some embodiments, spatial analysis of one type of analyte is performed independently of any other analysis. In some embodiments, spatial analysis is performed together for a plurality of types of analytes.

### Nucleic Acid and Nucleotide

As used herein, the terms “nucleic acid” and “nucleotide” are intended to be consistent with their use in the art and to include naturally-occurring species or functional analogs thereof. Particularly useful functional analogs of nucleic acids are capable of hybridizing to a nucleic acid in a sequence-specific fashion (e.g., capable of hybridizing to two nucleic acids such that ligation can occur between the two hybridized nucleic acids) or are capable of being used as a template for replication of a particular nucleotide sequence. Naturally-occurring nucleic acids generally have a backbone containing phosphodiester bonds. An analog structure can have an alternate backbone linkage including any of a variety of those known in the art. Naturally-occurring nucleic acids generally have a deoxyribose sugar (e.g., found in deoxyribonucleic acid (DNA)) or a ribose sugar (e.g., found in ribonucleic acid (RNA)).

A nucleic acid can contain nucleotides having any of a variety of analogs of these sugar moieties that are known in the art. A nucleic acid can include native or non-native nucleotides. In this regard, a native deoxyribonucleic acid can have one or more bases selected from the group consisting of adenine (A), thymine (T), cytosine (C), or guanine (G), and a ribonucleic acid can have one or more bases selected from the group consisting of uracil (U), adenine (A), cytosine (C), or guanine (G). Useful non-native bases that can be included in a nucleic acid or nucleotide are known in the art.

### Region of Interest

As used herein, the term “region of interest” generally refers to a region or area within a biological sample that is selected for specific analysis (e.g., a region in a biological sample that has morphological features of interest). A biological sample can have regions that show morphological feature(s) that may indicate the presence of disease or the development of a disease phenotype. For example, morphological features at a specific site within a tumor biopsy sample can indicate the aggressiveness, therapeutic resistance, metastatic potential, migration, stage, diagnosis, and/or prognosis of cancer in a subject. A change in the morphological features at a specific site within a tumor biopsy sample often correlate with a change in the level or expression of an analyte in a cell within the specific site, which can, in turn, be used to provide information regarding the aggressiveness, therapeutic resistance, metastatic potential, migration, stage, diagnosis, and/or prognosis of cancer in a subject. A region of interest in a biological sample can be used to analyze a specific area of interest within a biological sample, and thereby, focus experimentation and data gathering to a specific region of a biological sample (rather than an entire biological sample). This results in increased time efficiency of the analysis of a biological sample.

A region of interest can be identified in a biological sample using a variety of different techniques, e.g., expansion microscopy, bright field microscopy, dark field microscopy, phase contrast microscopy, electron microscopy, fluorescence microscopy, reflection microscopy, interference microscopy, and confocal microscopy, and combinations thereof. For example, the staining and imaging of a biological sample can be performed to identify a region of interest. In some examples, the region of interest can correspond to a specific structure of cytoarchitecture. In some embodiments, a biological sample can be stained prior to visualization to provide contrast between the different regions of the biological sample. The type of stain can be chosen depending on the type of biological sample and the region of the cells to be stained. In some embodiments, more than one stain can be used to visualize different aspects of the biological sample, e.g., different regions of the sample, specific cell structures (e.g., organelles), or different cell types. In other embodiments, the biological sample can be visualized or imaged without staining the biological sample.

In some examples, a region of interest can be removed from a biological sample and then the region of interest can be contacted to the substrate and/or array (e.g., as described herein). A region of interest can be removed from a biological sample using microsurgery, laser capture microdissection, chunking, a microtome, dicing, trypsinization, labelling, and/or fluorescence-assisted cell sorting.

### Subject

As used herein, the term “subject” refers to an animal, such as a mammal (e.g., human or a non-human simian), avian (e.g., bird), or other organism, such as a plant. Examples of subjects include, but are not limited to, a mammal such as a rodent, mouse, rat, rabbit, guinea pig, ungulate, horse, sheep, pig, goat, cow, cat, dog, primate (e.g., human or non-human primate); a plant such as Arabidopsis thaliana, corn, sorghum, oat, wheat, rice, canola, or soybean; an algae such as Chlamydomonas reinhardtii; a nematode such as Caenorhabditis elegans; an insect such as Drosophila melanogaster, mosquito, fruit fly, honey bee or spider; a fish such as zebrafish; a reptile; an amphibian such as a frog or Xenopus laevis; a Dictyostelium discoideum; a fungi such as Pneumocystis carinii, Takifugu rubripes, yeast, Saccharomyces cerevisiae or Schizosaccharomyces pombe; or a Plasmodium falciparum.

### Substrates

As used herein, a “substrate” refers to a support that is insoluble in aqueous liquid and that allows for positioning of biological samples, analytes, capture spots, and/or capture probes on the substrate. For instance, a substrate can be any surface onto which a sample and/or capture probes can be affixed (e.g., a chip, solid array, a bead, a slide, a coverslip, etc.). For the spatial analytical methods described in this section, a substrate is used to provide support to a biological sample, particularly, for example, a thin tissue section. In addition, in some embodiments, a substrate (e.g., the same substrate or a different substrate) functions as a support for direct or indirect attachment of capture probes to capture spots of the array.

A wide variety of different substrates can be used for the foregoing purposes. In general, a substrate can be any suitable support material. Exemplary substrates include, but are not limited to, glass, modified and/or functionalized glass, hydrogels, films, membranes, plastics (including e.g., acrylics, polystyrene, copolymers of styrene and other materials, polypropylene, polyethylene, polybutylene, polyurethanes, Teflon™, cyclic olefins, polyimides, etc.), nylon, ceramics, resins, Zeonor, silica or silica-based materials including silicon and modified silicon, carbon, metals, inorganic glasses, optical fiber bundles, and polymers, such as polystyrene, cyclic olefin copolymers (COCs), cyclic olefin polymers (COPs), polypropylene, polyethylene and polycarbonate.

The substrate can also correspond to a flow cell. Flow cells can be formed of any of the foregoing materials, and can include channels that permit reagents, solvents, capture spots, and molecules to pass through the flow cell.

The substrate can generally have any suitable form or format. For example, the substrate can be flat, curved, e.g., convexly or concavely curved towards the area where the interaction between a biological sample, e.g., tissue sample, and the substrate takes place. In some embodiments, the substrate is a flat, e.g., planar, chip or slide. The substrate can contain one or more patterned surfaces within the substrate (e.g., channels, wells, projections, ridges, divots, etc.). A substrate can be of any desired shape. For example, a substrate can be typically a thin, flat shape (e.g., a square or a rectangle). In some embodiments, a substrate structure has rounded corners (e.g., for increased safety or robustness). In some embodiments, a substrate structure has one or more cut-off corners (e.g., for use with a slide clamp or cross-table). In some embodiments, where a substrate structure is flat, the substrate structure can be any appropriate type of support having a flat surface (e.g., a chip or a slide such as a microscope slide).

In some embodiments, a substrate includes one or more markings on a surface of the substrate, e.g., to provide guidance for correlating spatial information with the characterization of the analyte of interest. For example, a substrate can be marked with a grid of lines (e.g., to allow the size of objects seen under magnification to be easily estimated and/or to provide reference areas for counting objects). In some embodiments, fiducials (e.g., fiducial markers, fiducial spots, or fiducial patterns) can be included on the substrate. Fiducials can be made using techniques including, but not limited to, printing, sand-blasting, and depositing on the surface. In some embodiments, the substrate (e.g., or a bead or a capture spot on an array) includes a plurality of oligonucleotide molecules (e.g., capture probes). In some embodiments, the substrate includes tens to hundreds of thousands or millions of individual oligonucleotide molecules (e.g., at least about 10,000, 50,000, 100,000, 500,000, 1,000,000, 10,000,000, 100,000,000, 1,000,000,000 or 10,000,000,000 oligonucleotide molecules). In some embodiments a substrate can include a substrate identifier, such as a serial number.

Further examples of substrates, including for example fiducial markers on such substrates, are disclosed in PCT publication 202020176788A1, entitled “Profiling of biological analytes with spatially barcoded oligonucleotide arrays”; as well as U.S. Pat. No. 11,501,440; U.S. Patent Publication No. US20210150707A1; U.S. Pat. No. 11,514,575, and U.S. Patent Publication No. US20210155982A1, each of which is hereby incorporated herein by reference in its entirety, each of which is hereby incorporated herein by reference in its entirety.

Spatial Analyte Data. As used herein, “spatial analyte data” refers to any data measured, either directly, from the capture of analytes on capture probes, or indirectly, through intermediate agents disclosed herein that bind to analytes in a sample, e.g., connected probes disclosed herein, analyte capture agents or portions thereof (such as, e.g., analyte binding moieties and their associated analyte binding moiety barcodes). Spatial analyte data thus may, in some aspects, include two different labels from two different classes of barcodes. One class of barcode identifies the analyte, while the other class of barcodes identifies the specific capture probe in which an analyte was detected.

### (B) Methods for Spatial Analysis of Analytes

Array-based spatial analysis methods involve the transfer of one or more analytes from a biological sample to an array of capture spots on a substrate, each of which is associated with a unique spatial location on the array. Subsequent analysis of the transferred analytes includes determining the identity of the analytes and the spatial location of each analyte within the sample. The spatial location of each analyte within the sample is determined based on the capture spot to which each analyte is bound on the array, and the capture spot's relative spatial location within the array.

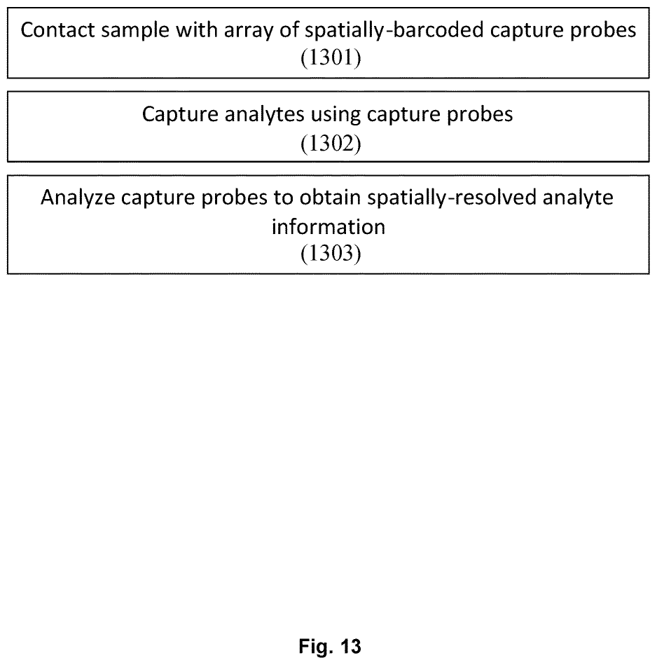

There are at least two general methods to associate a spatial barcode with one or more neighboring cells, such that the spatial barcode identifies the one or more cells, and/or contents of the one or more cells, as associated with a particular spatial location. One general method is to promote analytes out of a cell and towards the spatially-barcoded array. FIG. 13 depicts an exemplary embodiment of this general method. In FIG. 13, the spatially-barcoded array populated with capture probes (as described further herein) is contacted with a sample 1301, and the sample is permeabilized, allowing the target analyte to migrate away from the sample and toward the array 1302. The target analyte interacts with a capture probe on the spatially-barcoded array. Once the target analyte hybridizes to or is captured by the capture probe, the sample is optionally removed from the array and the capture probes are analyzed in order to obtain spatially-resolved analyte information 1303.

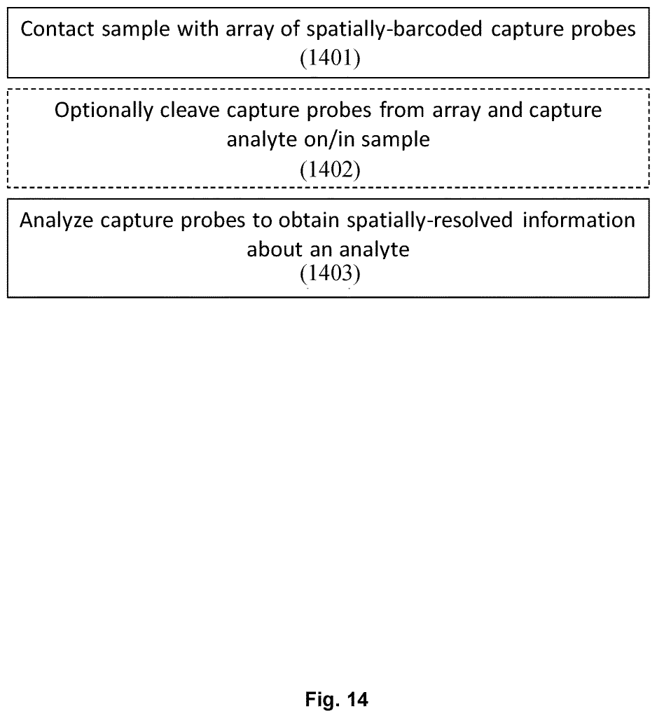

Another general method is to cleave the spatially-barcoded capture probes from an array and promote the spatially-barcoded capture probes towards and/or into or onto the sample. FIG. 14 depicts an exemplary embodiment of this general method, the spatially-barcoded array populated with capture probes (as described further herein) can be contacted with a sample 1401. The spatially-barcoded capture probes are cleaved and then interact with analytes within the cells within the provided sample 1402. The interaction can be a covalent or non-covalent cell-surface interaction. The interaction can be an intracellular interaction facilitated by a delivery system or a cell penetration peptide. Once the spatially-barcoded capture probe is associated with a particular analyte in a cell, the sample can be optionally removed. The sample can be optionally dissociated before analysis. Once the tagged analyte from the cell is associated with the spatially-barcoded capture probe, the capture probes can be analyzed to obtain spatially-resolved information about the tagged analyte in the cell 1403.

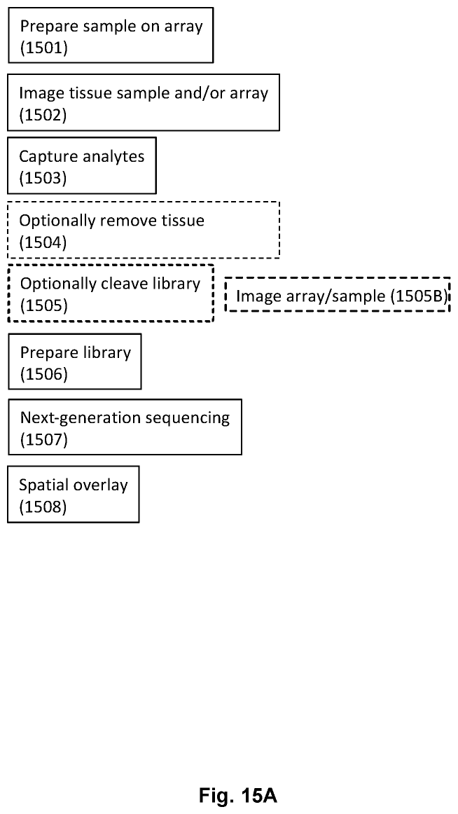

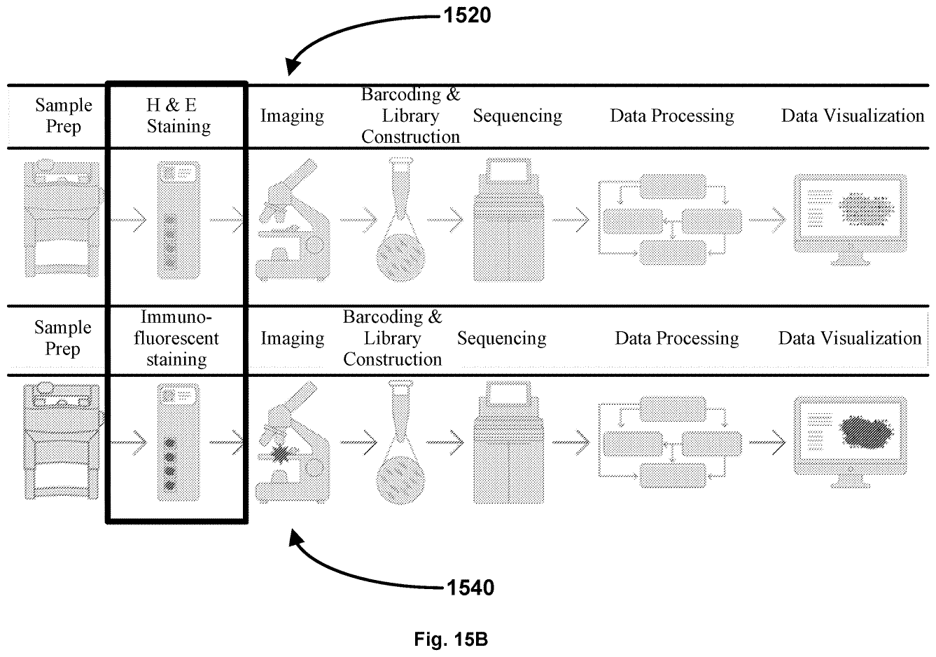

FIGS. 15A and 15B illustrate exemplary workflows that include preparing a sample on a spatially-barcoded array 1501. Sample preparation may include placing the sample on a substrate (e.g., chip, slide, etc.), fixing the sample, and/or staining the sample for imaging. The sample (stained or not stained) is then imaged on the array 1502 using bright-field (to image the sample, e.g., using a hematoxylin and eosin stain) or fluorescence (to image capture spots) as illustrated in the upper panel 1540 of FIG. 15B) and/or emission imaging modalities (as illustrated in the lower panel 1542 of FIG. 15B).

In some embodiments where the sample is analyzed with transcriptomics, along with the bright-field and/or emission imaging (e.g., fluorescence imaging), target analytes are released from the sample and capture probes forming a spatially-barcoded array hybridize or bind the released target analytes 1503. The sample can be optionally removed from the array 1504 and the capture probes can be optionally cleaved from the array 1505. Additionally, the targets can be migrated to the array from a non-arrayed substrate upon which is the biological sample. The sample and array are then optionally imaged a second time in both modalities 1505B while the analytes are reverse transcribed into cDNA, and an amplicon library is prepared 1506 and sequenced 1507. The images are spatially-overlaid in order to correlate spatially-identified sample information 1508. When the sample and array are not imaged a second time, 1505B, a spot coordinate file is supplied instead. The spot coordinate file replaces the second imaging step 1505B. Further, amplicon library preparation 1506 can be performed with a unique PCR adapter and sequenced 1507.

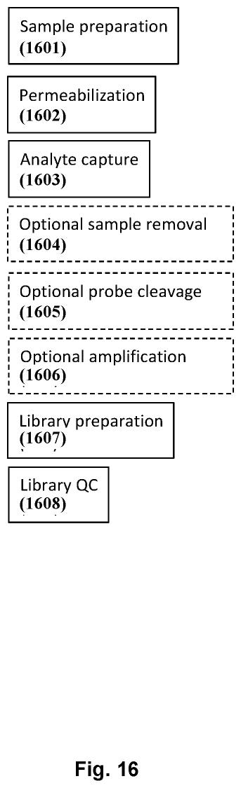

FIG. 16 shows another exemplary workflow that utilizes a spatially-barcoded array on a substrate (e.g., chip), where spatially-barcoded capture probes are located at areas called capture spots. The spatially-labelled capture probes can include a cleavage domain, one or more functional sequences, a spatial barcode, a unique molecular identifier, and a capture domain. The spatially-labelled capture probes can also include a 5′ end modification for reversible attachment to the substrate. The spatially-barcoded array is contacted with a sample 1601, and the sample is permeabilized through application of permeabilization reagents 1602. Permeabilization reagents may be administered by placing the array/sample assembly within a bulk solution. Alternatively, permeabilization reagents may be administered to the sample via a diffusion-resistant medium and/or a physical barrier such as a lid, where the sample is sandwiched between the diffusion-resistant medium and/or barrier and the array-containing substrate. The analytes are migrated toward the spatially-barcoded capture array using any number of techniques disclosed herein. For example, analyte migration can occur using a diffusion-resistant medium lid and passive migration (e.g., gravity). As another example, analyte migration can be active migration, using an electrophoretic transfer system, for example. Once the analytes are in close proximity to the spatially-barcoded capture probes, the capture probes can hybridize or otherwise bind a target analyte 1603. The sample can be optionally removed from the array 1604.

The capture probes can be optionally cleaved from the array 1605, and the captured analytes such as mRNA can be spatially-barcoded by performing a reverse transcriptase first strand cDNA reaction. A first strand cDNA reaction can be optionally performed using template switching oligonucleotides. For example, a template switching oligonucleotide can hybridize to a poly(C) tail added to a 3′ end of the cDNA by a reverse transcriptase enzyme. Template switching is described, for example, in U.S. Patent Application Publication Nos. US20210158522A1, US20210150707A1, US20210097684A1 and US20210155982A1, each of which is hereby incorporated herein by reference in its entirety. The original mRNA template and template switching oligonucleotide can be denatured from the cDNA and the spatially-barcoded capture probe can hybridize with the cDNA and second strand synthesis resulting in a complement of the cDNA can be generated. The first strand cDNA can be purified and collected for downstream amplification steps. The first strand cDNA can be optionally amplified using PCR 1606, where the forward and reverse primers flank the spatial barcode and target analyte regions of interest, generating a library associated with a particular spatial barcode 1607. In some embodiments, the library preparation can be quantified and/or subjected to quality control to verify the success of the library preparation steps 1608. In some embodiments, the cDNA comprises a sequencing by synthesis (SBS) primer sequence. The library amplicons are sequenced and analyzed to decode spatial information 1607, with an additional library quality control (QC) step 1608.

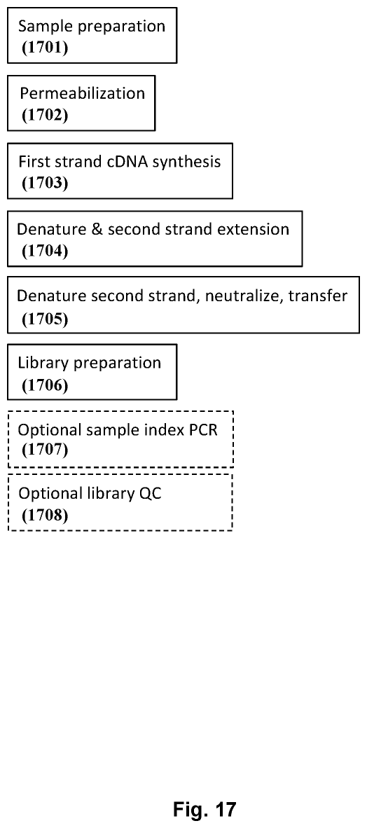

FIG. 17 depicts an exemplary workflow where the sample is removed from the spatially-barcoded array and the spatially-barcoded capture probes are removed from the array for barcoded analyte amplification and library preparation. Another embodiment includes performing first strand synthesis using template switching oligonucleotides on the spatially-barcoded array without cleaving the capture probes. In this embodiment, sample preparation 1701 and permeabilization 1702 are performed as described elsewhere herein. Once the capture probes capture the target analyte(s), first strand cDNA created by template switching and reverse transcriptase 1703 is then denatured and the second strand is then extended 1704. The second strand cDNA is then denatured from the first strand cDNA, neutralized, and transferred to a tube 1705. cDNA quantification and amplification can be performed using standard techniques discussed herein. The cDNA can then be subjected to library preparation 1706 and indexing 1707, including fragmentation, end-repair, and a-tailing, and indexing PCR steps. The library can also be optionally tested for quality control (QC) 1708.

Further details and non-limiting embodiments relating to methods for spatial analysis of analytes in biological samples, including removal of a sample from the array, release and amplification of analytes, analysis of captured analytes (e.g., by sequencing and/or multiplexing), and spatial resolution of analyte information (e.g., using lookup tables) are described in U.S. Pat. No. 11,501,440; U.S. Patent Publication No. US20210150707A1; U.S. Pat. No. 11,514,575, and U.S. Patent Publication No. US20210155982A1, each of which is hereby incorporated herein by reference in its entirety, each of which is hereby incorporated herein by reference in its entirety.

### II. Exemplary System Embodiments

Now that a general summary of the methods and terminology has been presented, detailed descriptions of various implementations of the present disclosure will now be described in conjunction with the figures.

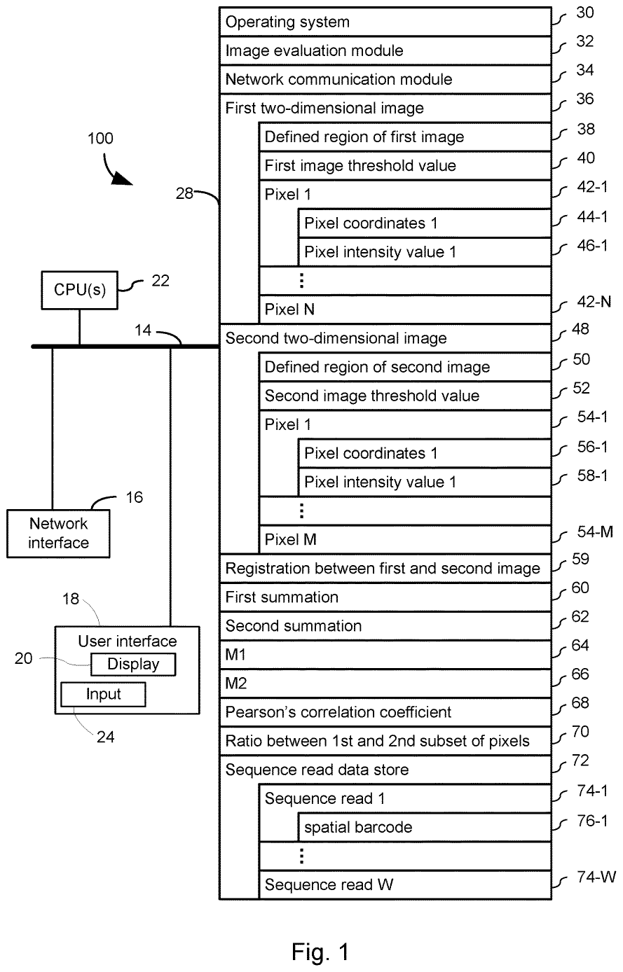

FIG. 1 illustrates a block diagram illustrating an exemplary, non-limiting system for colocalization of analyte expression data in accordance with some implementations. The system 100 in some implementations includes one or more processing units CPU(s) 22 (also referred to as processors), one or more network interfaces 16, a user interface 18, a memory 28, and one or more communication buses 14 for interconnecting these components. The one or more communication buses 14 optionally include circuitry (sometimes called a chipset) that interconnects and controls communications between system components. The memory 28 typically includes high-speed random access memory, such as DRAM, SRAM, DDR RAM, ROM, EEPROM, flash memory, CD-ROM, digital versatile disks (DVD) or other optical storage, magnetic cassettes, magnetic tape, magnetic disk storage or other magnetic storage devices, other random access solid state memory devices, or any other medium which can be used to store desired information; and optionally includes non-volatile memory, such as one or more magnetic disk storage devices, optical disk storage devices, flash memory devices, or other non-volatile solid state storage devices. The memory 28 optionally includes one or more storage devices remotely located from the CPU(s) 22. The memory 28, or alternatively the non-volatile memory device(s), comprises a non-transitory computer readable storage medium. It will be appreciated that this memory 28 can be distributed across one or more computers. In some implementations, the memory 28 or alternatively the non-transitory computer readable storage medium stores the following programs, modules and data structures, or a subset thereof:
an optional operating system 30, which includes procedures for handling various basic system services and for performing hardware dependent tasks;
an optional network communication module (or instructions) 34 for connecting the device 100 with other devices, or a communication network;
a first two-dimensional image 36, encoding spatial analyte unique molecular identifier (UMI) abundance data for a nucleic acid analyte from a biological sample, the first image comprising a defined region 38 that comprises a first plurality of pixels, each respective pixel 44 defined by corresponding two-dimensional coordinates 44 and a corresponding pixel intensity value 46, the first image further associated with a first image threshold value 40;
a second two-dimensional image 48, encoding spatial analyte abundance data for a non-nucleic acid analyte from the biological sample, the second image comprising a defined region 50 that comprises a second plurality of pixels, each respective pixel 54 defined by corresponding two-dimensional coordinates 56 and a corresponding pixel intensity value 58, the first image further associated with a first image threshold value 52;
a registration 59 between the image 36 and image 48 that allows for the comparison of corresponding pixels between the image 36 and the image 48;
a first summation 60 of the respective UMI abundance value of each respective pixel in a first subset of the first plurality of pixels in which the corresponding pixel in the first subset of the second plurality of pixels exceeds the threshold value 52;
a second summation 62 of each UMI abundance value of each pixel in the first subset of the first plurality of pixels;
a Pearson's correlation coefficient 68 from (i) the intensity of each pixel in the first subset of the first plurality of pixels, (ii) the intensity of each pixel in the second image corresponding to a pixel in the first subset of the first plurality of pixels, (iii) a first mean value across the first subset of the first plurality of pixels, and (iv) a second mean value across the pixels in the second image corresponding to the first subset of pixels in the first plurality of pixels;
a ratio 70 between a first subset of pixels in image 36 and a second subset of pixels in image 48; and
a sequence data store 72 comprising a plurality of sequence reads, each sequence read 74 including a spatial barcode 76.

In some implementations, the user interface 18 includes an input device (e.g., a keyboard, a mouse, a touchpad, a track pad, and/or a touch screen) 24 for a user to interact with the system 100 and a display 20.

In some implementations, one or more of the above identified elements are stored in one or more of the previously mentioned memory devices and correspond to a set of instructions for performing a function described above. The above identified modules or programs (e.g., sets of instructions) need not be implemented as separate software programs, procedures or modules, and thus various subsets of these modules may be combined or otherwise re-arranged in various implementations. In some implementations, the memory 28 optionally stores a subset of the modules and data structures identified above. Furthermore, in some embodiments, the memory stores additional modules and data structures not described above. In some embodiments, one or more of the above identified elements is stored in a computer system, other than that of system 100, that is addressable by system 100 so that system 100 may retrieve all or a portion of such data when needed.

Although FIG. 1 shows an exemplary system 100, the figure is intended more as functional description of the various features that may be present in computer systems than as a structural schematic of the implementations described herein. In practice, and as recognized by those of ordinary skill in the art, items shown separately could be combined and some items could be separated.

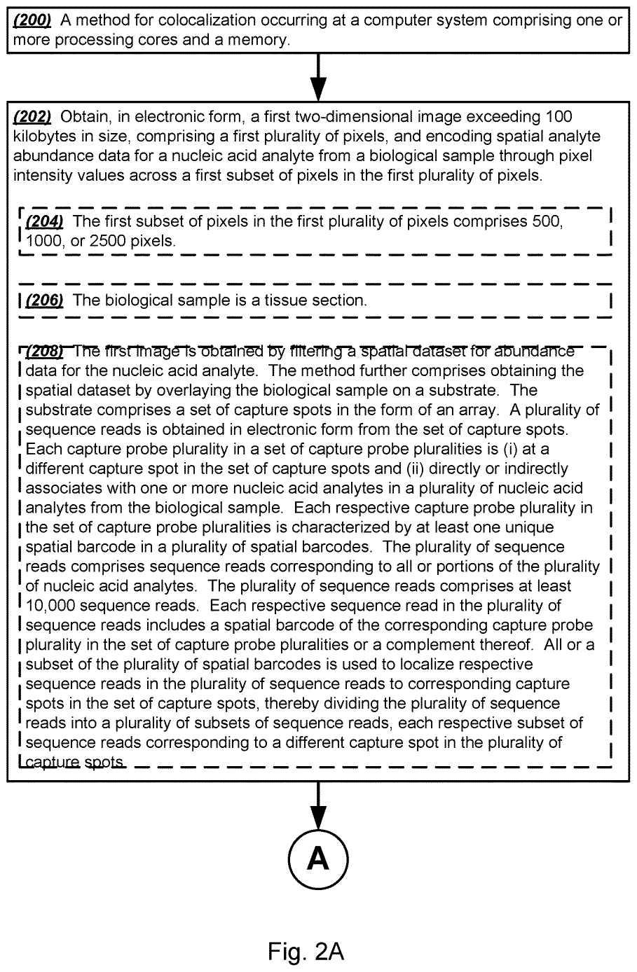

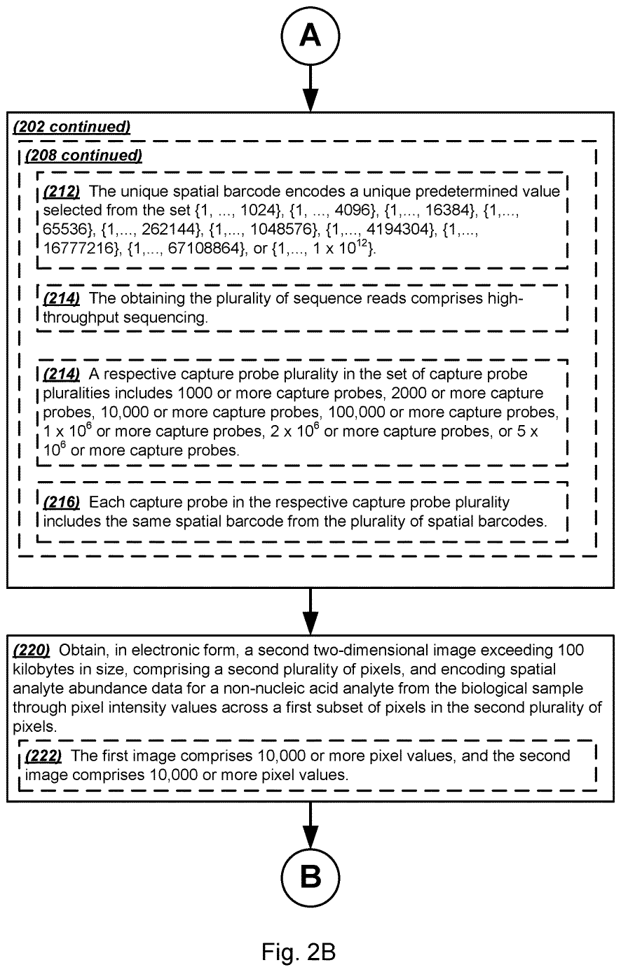

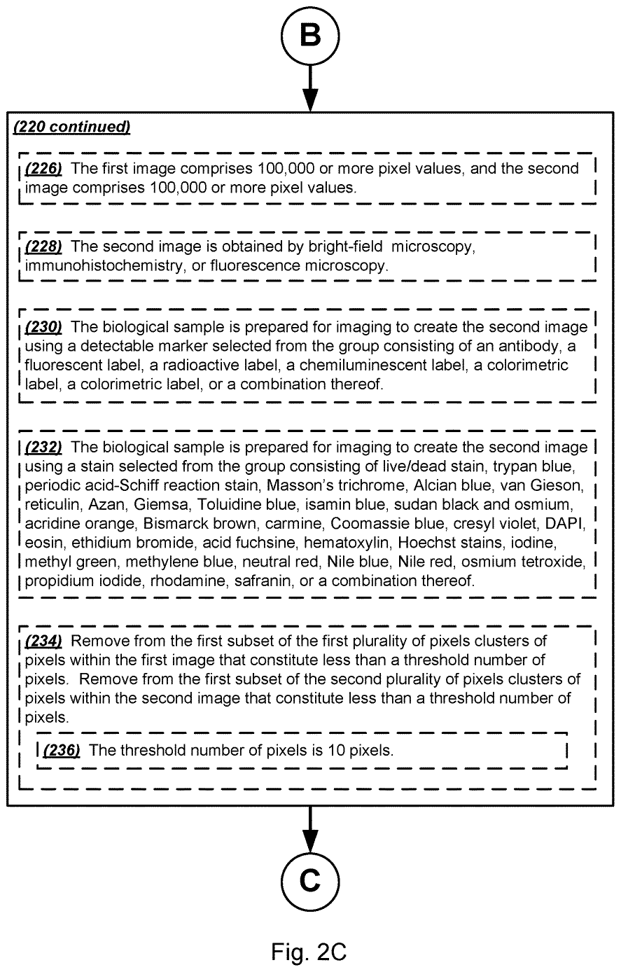

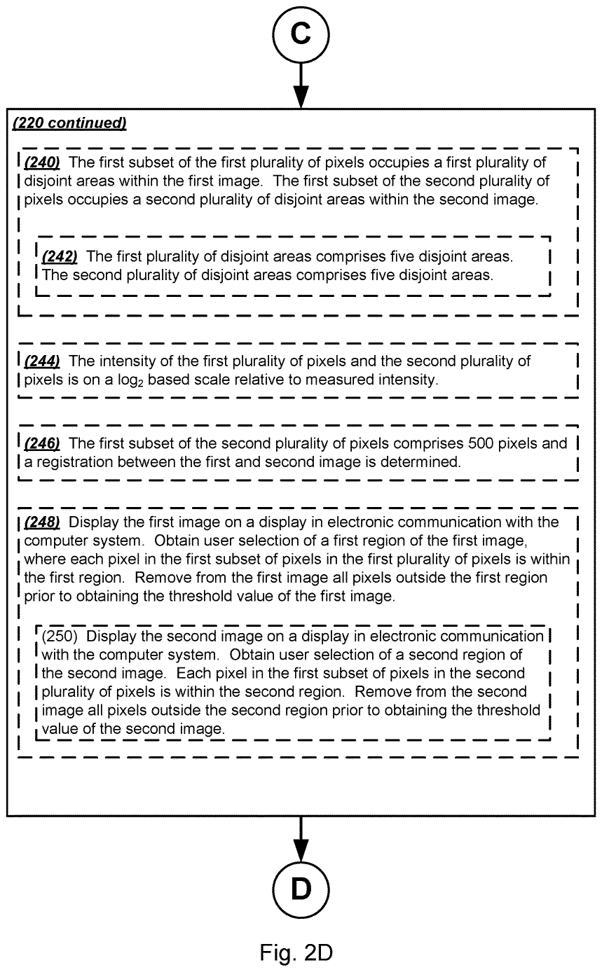

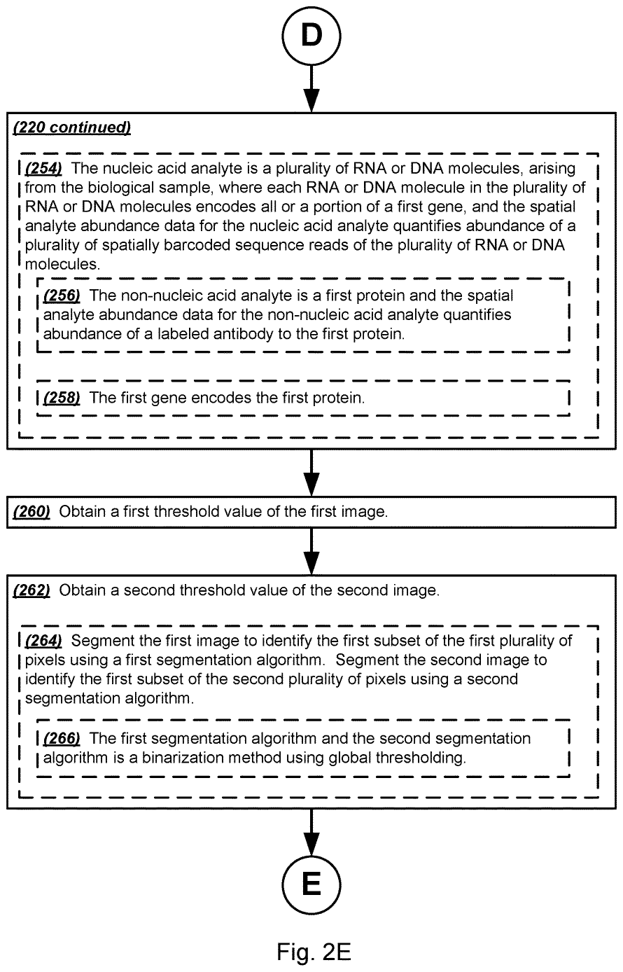

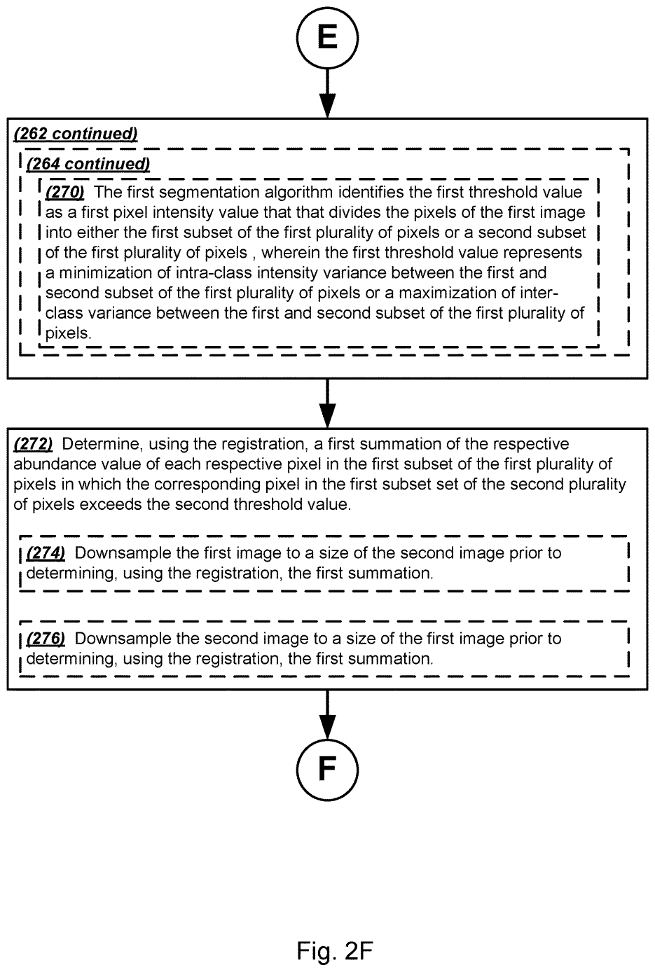

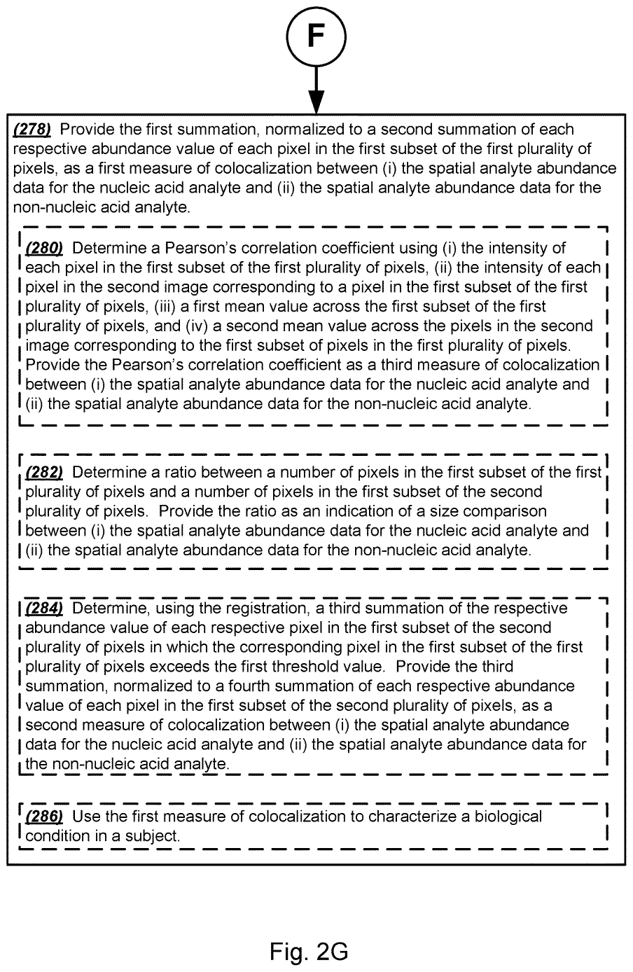

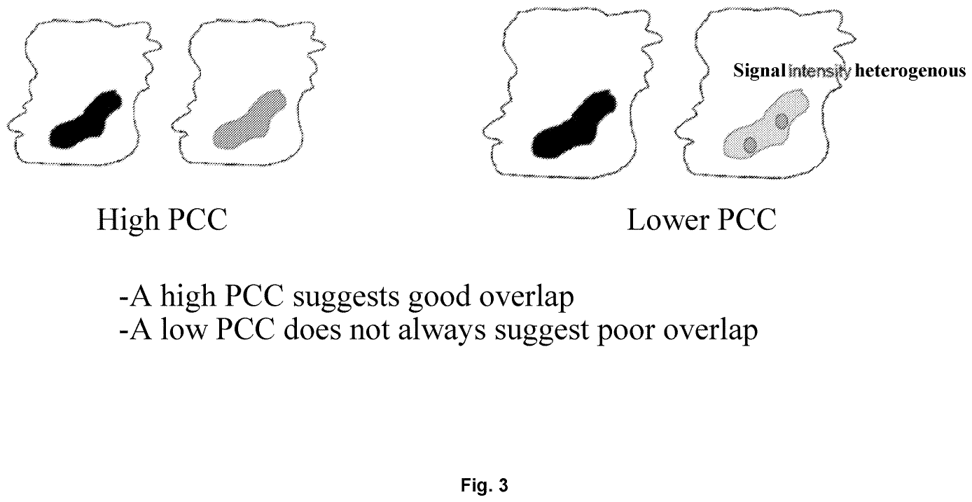

While a system in accordance with the present disclosure has been disclosed with reference to FIG. 1, example methods in accordance with the present disclosure are now detailed with reference to FIGS. 2A, 2B, 2C, 2D, 2E, 2F, and 2G.

### III. Specific Embodiments

This disclosure also provides methods and systems for colocalization between (i) the spatial analyte UMI abundance data or gene expression data for a nucleic acid analyte and (ii) the spatial analyte abundance data or expression data for a non-nucleic acid analyte. Provided below are detailed descriptions and explanations of various embodiments of the present disclosure. These embodiments are non-limiting and do not preclude any alternatives, variations, changes, and substitutions that can occur to those skilled in the art from the scope of this disclosure.

Block 200. Referring to block 200 of FIG. 2A, the present disclosure provides a method for colocalization occurring at a computer system 100, where the computer system comprises one or more processing cores 22 and a memory 28.

### Blocks 202-218.

Referring to block 202 of FIG. 2A, there is obtained, in electronic form, a first two-dimensional image. In some embodiments the first two-dimensional image exceeds 10, 20, 30, 40, 100, 500 or 900 kilobytes in size. The first two-dimensional image comprises a first plurality of pixels. The first two-dimensional image encodes spatial analyte unique molecular identifier (UMI) abundance data or gene expression data for a nucleic acid analyte from a biological sample of a subject through pixel intensity values across a first subset of pixels in the first plurality of pixels. Referring to block 204 of FIG. 2A, in some such embodiments, the first subset of pixels in the first plurality of pixels comprises 50, 100, 250, 500, 1000, or 2500 pixels.

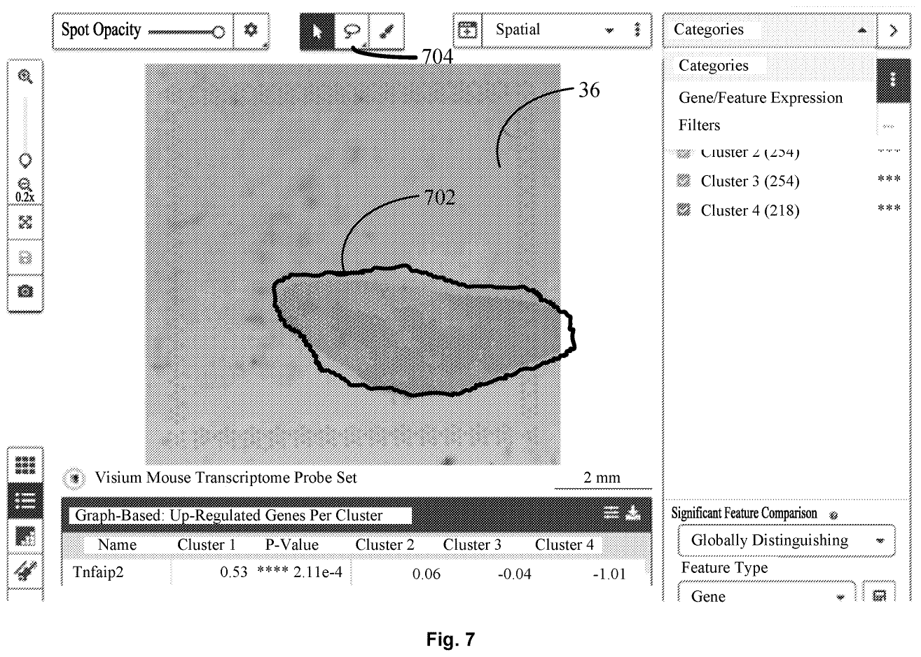

FIG. 7 illustrates obtaining, in electronic form, the first two-dimensional image 36. In some embodiments the first two-dimensional image 36 is superimposed on a picture of the substrate with the tissue sample on it. In some embodiments the picture of the substrate with the tissue sample on it is part of the first image. The first plurality of pixels include those pixels that represent UMI counts for the nucleic acid analyte. In FIG. 7, region 702 includes pixels that that represent UMI counts for the nucleic acid analyte.

Referring to block 206 of FIG. 2A, in some such embodiments the biological sample is a tissue section. In some embodiments, the biological sample is a sectioned tissue sample having a depth of 500 microns or less. In some embodiments, the biological sample is a sectioned tissue sample having a depth of 100 microns or less. In some embodiments, the sectioned tissue sample has a depth of 80 microns or less, 70 microns or less, 60 microns or less, 50 microns or less, 40 microns or less, 25 microns or less, 20 microns or less, 15 microns or less, 10 microns or less, 5 microns or less, 2 microns or less, or 1 micron or less. In some embodiments, the biological sample is a sectioned tissue sample having a depth of at least 0.1 microns, at least 1 micron, at least 5 microns, at least 10 microns, at least 15 microns, at least 20 microns, at least 30 microns, at least 50 microns, or at least 80 microns. In some embodiments, the sectioned tissue sample has a depth of between 10 microns and 20 microns, between 1 and 10 microns, between 0.1 and 5 microns, between 20 and 100 microns, between 1 and 50 microns, or between 0.5 and 10 microns. In some embodiments, the sectioned tissue sample falls within another range starting no lower than 0.1 microns and ending no higher than 500 microns. Further embodiments of tissue sections are provided herein (see, “Definitions: (A) General Definitions: Biological Samples,” above).

In some embodiments, the biological sample is a plurality of cells. In some embodiments, for instance, the biological sample is a plurality of spatially arrayed cells. Examples of suitable biological samples contemplated for use in the present disclosure are described in further detail herein (see, “Definitions: (A) General Definitions: Biological Samples,” above).

In some embodiments, the biological sample is attached (e.g., mounted) onto a substrate. In some embodiments, the substrate comprises a sample area onto which the sample is placed. In some embodiments, the substrate further includes a sample area indicator identifying the sample area. In some embodiments, the substrate includes one or more fiducials. For instance, in some embodiments, fiducials are used to aid alignment of a sample area on the substrate with an image of a sample. In some embodiments, the substrate does not include fiducials. Example suitable embodiments for substrates that are contemplated for use in the present disclosure include any of the embodiments described herein, such as those disclosed above (see, “Definitions: (A) General Definitions: Substrates”) and in PCT publication 202020176788A1, entitled “Profiling of biological analytes with spatially barcoded oligonucleotide arrays”; and U.S. Pat. No. 11,501,440; U.S. Patent Publication No. US20210150707A1; U.S. Pat. No. 11,514,575, and U.S. Patent Publication No. US20210155982A1, each of which is hereby incorporated herein by reference in its entirety, each of which is hereby incorporated herein by reference in its entirety.

In some embodiments, the one or more fiducials comprise any suitable indicator that denotes a point of reference on the substrate (e.g., chip). In some embodiments, the one or more fiducials comprise one or more fiducial marks. Examples of suitable spatial fiducials contemplated for use in the present disclosure are described in further detail herein (see, “Definitions: (A) General Definitions: Spatial fiducials,” above).

Referring to block 208 of FIG. 2A, in some embodiments the first image is obtained by filtering a spatial dataset for UMI abundance data for the nucleic acid analyte. The method further comprises obtaining the spatial dataset by overlaying the biological sample on a substrate. The substrate comprises a set of capture spots in the form of an array.

In some embodiments, the set of capture spots comprises at least 50, at least 100, at least 200, at least 300, at least 400, at least 500, at least 1000, at least 2000, at least 3000, at least 4000, at least 5000, at least 10,000, at least 15,000, at least 20,000, or at least 40,000 capture spots. In some embodiments, the set of capture spots consists of no more than 100,000, no more than 50,000, no more than 20,000, no more than 10,000, no more than 5000, no more than 1000, no more than 500, or no more than 100 capture spots. In some embodiments, the set of capture spots consists of from 100 to 500, between 500 and 1000, from 1000 to 5000, from 5000 to 10,000, from 10,000 to 15,000, or from 15,000 to 20,000 capture spots. In some embodiments, the set of capture spots falls within another range starting no lower than 50 capture spots and ending no higher than 100,000 capture spots.

In some embodiments, each respective capture spot in the set of capture spots includes a plurality of capture probes. In some embodiments, the plurality of capture probes includes 500 or more, 1000 or more, 2000 or more, 3000 or more, 5000 or more, 10,000 or more, 20,000 or more, 30,000 or more, 50,000 or more, 100,000 or more, 500,000 or more, 1×10⁶ or more, 2×10⁶ or more, or 5×10⁶ or more capture probes. In some embodiments, the plurality of capture probes consists of no more than 1×10⁷, no more than 1×10⁶, no more than 100,000, no more than 50,000, no more than 10,000, no more than 5000, no more than 2000, or no more than 1000 capture probes. In some embodiments, the plurality of capture probes is from 500 to 10,000, from 5000 to 100,000, from 1000 to 1×10⁶, from 10,000 to 500,000, or from 1×10⁶ to 1×10⁷ capture probes. In some embodiments, the plurality of capture probes falls within another range starting no lower than 500 capture probes and ending no higher than 1×10⁷ capture probes.

In some embodiments, a respective capture spot comprises any area of any two- or three-dimensional geometry (e.g., of any shape). For instance, in some embodiments, a respective capture spot is circular. In some embodiments, a respective capture spot is not circular. In some embodiments, the set of capture spots is positioned on a respective substrate (e.g., the second substrate) in a specific arrangement. In some such embodiments, the set of capture spots is provided as a capture spot array.

Numerous additional embodiments of capture domain types, capture spot sizes, arrays, probes, spatial barcodes, analytes, and/or other features of capture spots including but not limited to dimensions, designs, and modifications, and any substitutions and/or combinations thereof, are discussed in detail at length above (e.g., in “Definitions: (A) General Definitions: Capture Probes,” “Definitions: (A) General Definitions: Capture spots,” and “Definitions: (A) General Definitions: Capture spot arrays,” above).

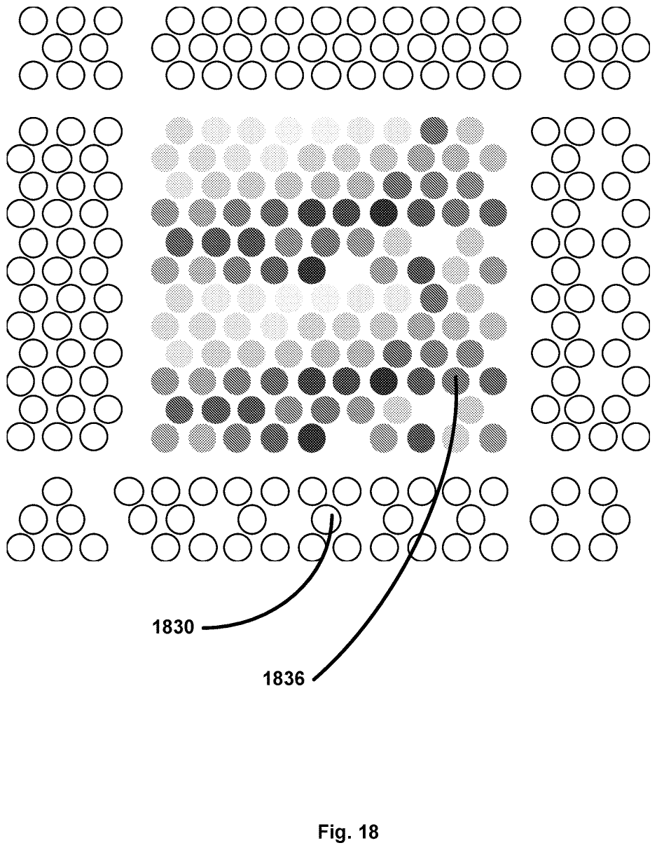

For instance, FIG. 18 illustrates a substrate (e.g., a chip) that has a plurality of spatial fiducials 1830 and a set of capture spots 1836, in accordance with an embodiment of the present disclosure.

The plurality of sequence reads is obtained in electronic form from the set of capture spots. Each capture probe plurality in a set of capture probe pluralities is (i) at a different capture spot in the set of capture spots and (ii) directly or indirectly associates with one or more nucleic acid analytes in a plurality of nucleic acid analytes from the biological sample. Each respective capture probe plurality in the set of capture probe pluralities is characterized by at least one unique spatial barcode in a plurality of spatial barcodes. The plurality of sequence reads comprises sequence reads corresponding to all or portions of the plurality of nucleic acid analytes. In some embodiments, the plurality of sequence reads comprises at least 1,000, 5,000, 10,000, 100,000, or 1×10⁶ sequence reads. Each respective sequence read in the plurality of sequence reads includes a spatial barcode of the corresponding capture probe plurality in the set of capture probe pluralities or a complement thereof as well as a unique molecular identifier (UMI) that associates the respective sequence read to a particular nucleic acid in the biological sample. All or a subset of the plurality of spatial barcodes is used to localize respective sequence reads in the plurality of sequence reads to corresponding capture spots in the set of capture spots, thereby dividing the plurality of sequence reads into a plurality of subsets of sequence reads, each respective subset of sequence reads corresponding to a different capture spot in the plurality of capture spots. In such embodiments, each respective pixel or group of pixels of the first image is associated with a particular capture spot and the intensity of the respective pixel or group of pixels is determined by a count of unique UMIs for the nucleic acid of block 202 measured in the particular capture spot.

Referring to block 212 of FIG. 2B, in some such embodiments, the unique spatial barcode encodes a unique predetermined value selected from the set 1, . . . , 1024}, {1, . . . , 4096}, {1, . . . , 16384}, {1, . . . , 65536}, {1, . . . , 262144}, {1, . . . , 1048576}, {1, . . . , 4194304}, {1, . . . , 16777216}, {1, . . . , 67108864}, or {1, . . . , 1×10¹²}. In some embodiments, the plurality of spatial barcodes is used to localize respective sequence reads in the plurality of sequence reads to corresponding capture spots in the set of capture spots, thereby dividing a plurality of sequence reads of a respective image into a plurality of subsets of sequence reads. Each respective subset of sequence reads corresponds to a different capture spot in the plurality of capture spots.

In some embodiments, the plurality of spatial barcodes is used to localize respective sequence reads in the plurality of sequence reads to corresponding regions of the substrate, where such regions have a resolution on the scale of micrometers (e.g., 100 microns or less, 80 microns or less, 50 microns or less, 40 microns or less, or 20 microns or less). That is, in some embodiments, capture spots are not implemented on the substrate.

Referring to block 214 of FIG. 2B, in some embodiments, the obtaining the plurality of sequence reads comprises high-throughput sequencing. A wide variety of different sequencing methods can be used to obtain the plurality of sequence reads. In general, sequence reads can be obtained from, for example, nucleic acid molecules such as deoxyribonucleic acid (DNA) or ribonucleic acid (RNA), including variants or derivatives thereof (e.g., single stranded DNA or DNA/RNA hybrids, and nucleic acid molecules with a nucleotide analog). Sequencing can be performed by various commercial systems. More generally, sequencing can be performed using nucleic acid amplification, polymerase chain reaction (PCR) (e.g., digital PCR and droplet digital PCR (ddPCR), quantitative PCR, real time PCR, multiplex PCR, PCR-based singleplex methods, emulsion PCR), and/or isothermal amplification.

Other examples of methods for sequencing include, but are not limited to, DNA hybridization methods (e.g., Southern blotting), restriction enzyme digestion methods, Sanger sequencing methods, next-generation sequencing methods (e.g., single-molecule real-time sequencing, nanopore sequencing, and Polony sequencing), ligation methods, and microarray methods. Additional examples of sequencing methods that can be used include targeted sequencing, single molecule real-time sequencing, exon sequencing, electron microscopy-based sequencing, panel sequencing, transistor-mediated sequencing, direct sequencing, random shotgun sequencing, Sanger dideoxy termination sequencing, whole-genome sequencing, sequencing by hybridization, pyrosequencing, capillary electrophoresis, gel electrophoresis, duplex sequencing, cycle sequencing, single-base extension sequencing, solid-phase sequencing, high-throughput sequencing, massively parallel signature sequencing, co-amplification at lower denaturation temperature-PCR (COLD-PCR), sequencing by reversible dye terminator, paired-end sequencing, near-term sequencing, exonuclease sequencing, sequencing by ligation, short-read sequencing, single-molecule sequencing, sequencing-by-synthesis, real-time sequencing, reverse-terminator sequencing, nanopore sequencing, 454 sequencing, Solexa Genome Analyzer sequencing, SOLID™ sequencing, MS-PET sequencing, and any combinations thereof.

In some embodiments, the plurality of sequence reads comprises 10,000 or more sequence reads, 50,000 or more sequence reads, 100,000 or more sequence reads, or 1×10⁶ or more sequence reads. In some embodiments, the plurality of sequence reads comprises at least 100,000, at least 200,000, at least 500,000, at least 800,000, at least 1×10⁶, at least 2×10⁶, at least 5×10⁶, at least 8×10⁶, at least 1×10⁷, or at least 1×10⁸ sequence reads. In some embodiments, the plurality of sequence reads comprises no more than 1×10⁹, no more than 1×10⁸, no more than 1×10⁷, no more than 1×10⁶, no more than 500,000, no more than 200,000 or no more than 100,000 sequence reads. In some embodiments, the plurality of sequence reads consists of from 10,000 to 1×10⁷, from 100,000 to 1×10⁸, from 1×10⁵ to 1×10⁸, or from 10,000 to 500,000 sequence reads. In some embodiments, the plurality of sequence reads falls within another range starting no lower than 10,000 sequence reads and ending no higher than 1×10⁹ sequence reads. In practice, only those sequence reads that map to a sequence of the nucleic acid analyte are of interest. Thus, the plurality of sequence reads is filtered for those sequences that uniquely map to a sequence of the nucleic acid analyte are of interest. In some embodiments the nucleic acid analyte of interest has a sequence of at least 20, 30, 40, 50, 60, 80, 100, 150, 200, 250, 300, 350, or 400 nucleic acids. In some embodiments the sequence read includes a contiguous sequence of at least 60 percent, 70 percent, 80 percent, 90 percent or all of the sequence of the nucleic acid analyte of interest. In some embodiments less than 40 percent, less than 30 percent, or less than 20 percent of the sequence reads in the plurality of sequence reads map to the sequence of the nucleic acid analyte of interest by such a criteria.

In some embodiments, the plurality of sequence reads is obtained using a sequencing device such as, without limitation, a sequencing system by Illumina®, Pacific Biosciences (PacBio®), Oxford Nanopore®, or Life Technologies (Ion Torrent®). Alternatively or in addition, the plurality of sequence reads may be obtained by sequencing using nucleic acid amplification, polymerase chain reaction (PCR) (e.g., digital PCR, quantitative PCR, or real time PCR), or isothermal amplification. In some examples, such systems provide sequencing reads (also “reads” herein). A read may include a string of nucleic acid bases corresponding to a sequence of a nucleic acid molecule that has been sequenced. Apparatuses suitable for obtaining the plurality of sequence reads are further described in, e.g., U.S. Patent Application No. 63/080,547, entitled “Sample Handling Apparatus and Image Registration Methods,” filed Sep. 18, 2020, U.S. Patent Application No. 63/080,514, entitled “Sample Handling Apparatus and Fluid Delivery Methods,” filed Sep. 18, 2020, U.S. Patent Application No. 63/155,173, entitled “Sample Handling Apparatus and Image Registration Methods,” filed Mar. 1, 2021, and PCT Application No. US2019/065100, entitled “Imaging system hardware,” filed Dec. 6, 2019, each of which is hereby incorporated by reference herein in its entirety.

Referring to block 216 of FIG. 2B, in some embodiments a respective capture probe plurality in the set of capture probe pluralities includes 1000 or more capture probes, 2000 or more capture probes, 10,000 or more capture probes, 100,000 or more capture probes, 1×10⁶ or more capture probes, 2×10⁶ or more capture probes, or 5×10⁶ or more capture probes comprises high-throughput sequencing. Referring to block 218 of FIG. 2B, in some such embodiments each capture probe in the respective capture probe plurality includes the same spatial barcode from the plurality of spatial barcodes. In some embodiments, each respective capture probe in the respective capture probe plurality includes a different and unique set of spatial barcodes from the plurality of spatial barcodes that is different than the spatial barcodes of any other capture probe in the capture probe plurality.

### Blocks 220-258.

Referring to block 220 of FIG. 2B, there is also obtained, in electronic form, a second two-dimensional image. In some embodiments the second two-dimensional image exceeds 10, 20, 30, 40, 100, 500 or 900 kilobytes in size. The second two-dimensional image comprises a second plurality of pixels. The second two-dimensional image encodes spatial analyte abundance data for a non-nucleic acid analyte from the biological sample through pixel intensity values across a first subset of pixels in the second plurality of pixels. In some embodiments, the first subset of pixels in the second plurality of pixels comprises 50, 100, 250, 500, 1000, or 2500 pixels.

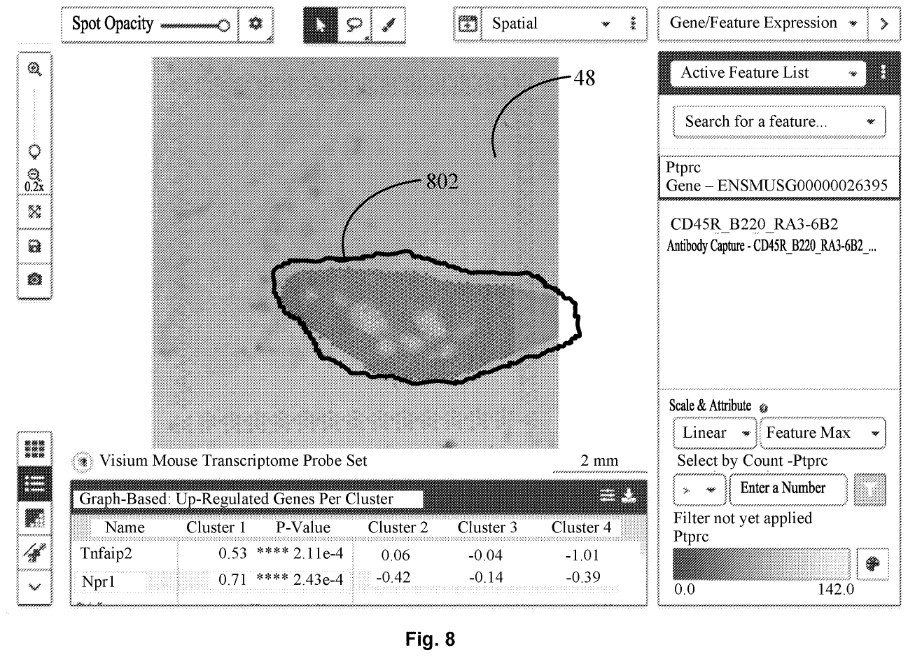

FIG. 8 illustrates obtaining, in electronic form, the second two-dimensional image 48. In some embodiments the second two-dimensional image 48 is superimposed on a picture of the substrate with the tissue sample on it. In some embodiments the picture of the substrate with the tissue sample on it is part of the second image. The second plurality of pixels includes those pixels that represent UMI counts for the non-nucleic acid analyte. In FIG. 8, region 802 includes the plurality of pixels that represent UMI counts for the non-nucleic acid analyte.

In some embodiments the second image is obtained by incubating the biological sample that is placed on a substrate (e.g., slide, chip, etc.) in which each capture area on the slide incorporates thousands of molecularly barcoded, spatially encoded capture spots (features). These capture spots (features) are functionalized with barcoded nucleic acid oligonucleotides that have complementary capture sequences to antibody-derived nucleic acid oligonucleotides. The biological sample on the substrate is incubated with a probe-based RNA panel followed by an immunology (oligonucleotide-tagged) antibody panel. Subsequently, the biological sample is permealized and paired gene and non-nucleic (e.g., protein) sequence read libraries are created for the biological sample. In some embodiments, the sequence reads from the probe-based RNA panel form the plurality of sequence reads that are used to create the first two-dimensional image. Each such sequence read includes (i) a spatial barcode that indicates where on the substrate the sequence read is associated and (ii) a unique molecular identifier (UMI) that is unique to a particular nucleic acid molecule in the biological sample. The first image, then, is a two-dimensional measurement of UMI count for a particular nucleic acid analyte of interest. The sequence reads from the oligonucleotide-tagged antibody panel form the plurality of sequence reads that are used to create the second two-dimensional image. Each such sequence read includes (i) a spatial barcode that indicates where on the substrate the sequence read is associated and (ii) a unique molecular identifier that is unique to a particular non-nucleic acid analyte (e.g., protein) in the biological sample. Moreover, the sequence of the sequence read indicates the identity of this protein. The second image, then, is a two-dimensional measurement of UMI count for a particular non-nucleic acid analyte of interest that is paired to the nucleic acid analyte of interest of the first image. Examples of such multi-model paired image generation are described in Uytingco et al., “Multiomic characterization of the tumor microenvironment in FFPE tissue by simultaneous protein and gene expression profiling,” Cancer Research, 2022—AACR, which is hereby incorporated by reference.

Referring to blocks 222 and 226 of FIGS. 2B and 2C, respectively, in some embodiments the first and second images each comprise 500 or more pixel values, 1000 or more pixel values, 2500 or more pixel values, 5000 or more pixel values, 10,000 or more pixel values, 50,000 or more pixel values, 100,000 or more pixel values, 500,000 or more pixel values, 1×10⁶ or more pixel values, 2×10⁶ or more pixel values, or 10×10⁶ or more pixel values, each pixel value for a different pixel in the image. In some embodiments, the first and second images each comprise at least 100, at least 500, at least 1000, at least 5000, at least 10,000, at least 50,000, at least 100,000, at least 500,000, at least 1×10⁶, at least 2×10⁶, at least 3×10⁶, at least 5×10⁶, at least 8×10⁶, at least 1×10⁷, at least 1×10⁸, at least 1×10⁹, at least 1×10¹⁰, or at least 1×10¹¹ pixels. In some embodiments, the first and second images each consist of no more than 1×10¹², no more than 1×10¹¹, no more than 1×10¹⁰, no more than 1×10⁹, no more than 1×10⁸, no more than 1×10⁷, no more than 1×10⁶, no more than 100,000, no more than 10,000, or no more than 1000 pixels. In some embodiments, the first and second images each consists of from 1000 to 100,000, from 10,000 to 500,000, from 100,000 to 1×10⁶, from 500,000 to 1×10⁹, or from 1×10⁶ to 1×10⁸ pixels. In some embodiments, the first and second images each include a plurality of pixels that falls within another range starting no lower than 100 pixels and ending no higher than 1×10¹² pixels.

In some embodiments the first or second two-dimensional image is in any electronic image file format, including but not limited to JPEG/JFIF, TIFF, Exif, PDF, EPS, GIF, BMP, PNG, PPM, PGM, PBM, PNM, WebP, HDR raster formats, HEIF, BAT, BPG, DEEP, DRW, ECW, FITS, FLIF, ICO, ILBM, IMG, PAM, PCX, PGF, JPEG XR, Layered Image File Format, PLBM, SGI, SID, CD5, CPT, PSD, PSP, XCF, PDN, CGM, SVG, PostScript, PCT, WMF, EMF, SWF, XAML, and/or RAW.

Additional suitable embodiments for obtaining and/or receiving images (e.g., the first two-dimensional image and/or the two-dimensional second image) that are contemplated for use in the present disclosure include any of the embodiments described herein, such as those disclosed above (see, “Definitions: (A) General Definitions: Imaging”) and Nos. US20210158522A1; US20210150707A1; US20210097684A1 and US20210155982A1, each of which is hereby incorporated herein by reference in its entirety. However, the first image represents UMI counts of the nucleic acid analyte rather than spectroscopic data.

In some embodiments, a respective image (e.g., the first two-dimensional image and/or the second two-dimensional image) is represented as an array (e.g., matrix) comprising a plurality of pixels, such that the location of each respective pixel in the plurality of pixels in the array (e.g., matrix) corresponds to its original location in the image. In some embodiments, a respective image is represented as a vector comprising a plurality of pixels, such that each respective pixel in the plurality of pixels in the vector comprises spatial information corresponding to its original location in the image.

In some embodiments, the plurality of pixels in a respective image (e.g., the first two-dimensional image and/or the second two-dimensional image) corresponds to the location of each capture spot in a set of capture spots on a substrate. In some embodiments, each capture spot in the set of capture spots is represented by five or more, ten or more, 100 or more, 1000 or more, 10,000 or more, 50,000 or more, 100,000 or more, or 200,000 or more contiguous pixels in a respective image. In some embodiments, each capture spot in the set of capture spots is represented by no more than 500,000, no more than 200,000, no more than 100,000, no more than 50,000, no more than 10,000, no more than 1000, or more than 100, no more than 50, no more than 20, or no more than 10 contiguous pixels in a respective image. In some embodiments, each capture spot is represented by between 8 and 250,000, between 8 and 500,000, between 8 and 100,000, or between 100 and 100,000 contiguous pixels in a respective image. In some embodiments, each capture spot is represented by another range of contiguous pixels in a respective image starting no lower than 5 pixels and ending no higher than 500,000 pixels.

In some embodiments, a respective image (e.g., the first image and/or the second image) has an image size between 1 KB and 1 MB, between 1 MB and 0.5 GB, between 0.5 GB and 5 GB, between 5 GB and 10 GB, or greater than 10 GB.

In some implementations, the second two-dimensional image is suitable for resolving subcellular histological and pathological features having a resolution less than 50 microns, less than 40 microns, less than 30 microns, less than 20 microns or between 5 microns and 10 microns.

In some embodiments, the second two-dimensional image is obtained using an image capture device, such as a microscope. In some embodiments, the second image is obtained by a high-resolution image capture device (e.g., a bright-field and/or fluorescent microscope). In some embodiments, the second image is obtained by a low-resolution image capture device attached to a sample handling apparatus.

In some embodiments, the first two-dimensional image and/or the second two-dimensional image is modified prior to use. In some embodiments, such image modification comprises adjusting a brightness of the two-dimensional image, adjusting a contrast of the two-dimensional image, flipping the two-dimensional image, rotating the two-dimensional image, cropping the two-dimensional image, zooming a view of the two-dimensional image, panning across the two-dimensional image, or overlaying a grid onto the respective two-dimensional image.

In some embodiments, the image modification comprises preprocessing the two-dimensional image. For example, in some embodiments, preprocessing is performed on the first two-dimensional image and/or the second two-dimensional image. In some embodiments, the preprocessing includes matching pixelwise resolution (upsampling), mirror image flipping, angular rotation, or any combination thereof. In some embodiments, an initial image transformation is generated based on an initial transform type and an initial transformation matrix. The initial transformation matrix type includes, in some implementations, a similarity transformation matrix based on translation, rotation, and scale. In some embodiments, the initial transformation matrix includes an affine transformation matrix based on translation, rotation, scale, and shear. Preprocessing of images is further described in, e.g., U.S. Patent Application No. 63/080,547, entitled “Sample Handling Apparatus and Image Registration Methods,” filed Sep. 18, 2020, U.S. Patent Application No. 63/080,514, entitled “Sample Handling Apparatus and Fluid Delivery Methods,” filed Sep. 18, 2020, U.S. Patent Application No. 63/155,173, entitled “Sample Handling Apparatus and Image Registration Methods,” filed Mar. 1, 2021, and PCT Application No. US2019/065100, entitled “Imaging system hardware,” filed Dec. 6, 2019, each of which is hereby incorporated by reference herein in its entirety.

In some embodiments, the second two-dimensional image, like the first two-dimensional image, is a two-dimensional UMI count of an analyte (here, the non-nucleic analyte of block 220). However, referring to block 228 of FIG. 2C, the present disclosure is not so limited. In some embodiments the second image is acquired by bright-field microscopy, immunohistochemistry, or fluorescence microscopy image. In some embodiments, the second image is obtained by immunofluorescence microscopy. More generally, any suitable imaging technique, and any substitutions, modifications, additions, deletions, and/or combinations thereof, as will be apparent to one skilled in the art can be used to obtain the second two-dimensional image. For example, in some embodiments, the second two-dimensional image is a histological image of the biological sample. In some embodiments, a histological image generally refers to any image that contains structural information for a biological sample and/or a biological tissue. In some embodiments, a histological image is obtained using any suitable stain, as described in further detail herein.

Referring to block 230 of FIG. 2C, in some embodiments the biological sample is prepared for imaging to create the second image using a detectable marker selected from the group consisting of an antibody, a fluorescent label, a radioactive label, a chemiluminescent label, a colorimetric label, a colorimetric label, or a combination thereof.

Referring to block 232 of FIG. 2C, in some embodiments biological sample is prepared for imaging to create the second two-dimensional image using a stain selected from the group consisting of live/dead stain, trypan blue, periodic acid-Schiff reaction stain, Masson' trichrome, Alcian blue, van Gieson, reticulin, Azan, Giemsa, Toluidine blue, isamin blue, sudan black and osmium, acridine orange, Bismarck brown, carmine, Coomassie blue, cresyl violet, DAPI, eosin, ethidium bromide, acid fuchsine, hematoxylin, Hoechst stains, iodine, methyl green, methylene blue, neutral red, Nile blue, Nile red, osmium tetroxide, propidium iodide, rhodamine, safranin, or a combination thereof.

In some embodiments the second two-dimensional image is acquired using transmission light microscopy (e.g., bright field transmission light microscopy, dark field transmission light microscopy, oblique illumination transmission light microscopy, dispersion staining transmission light microscopy, phase contrast transmission light microscopy, differential interference contrast transmission light microscopy, emission imaging, etc.). See, for example, Methods in Molecular Biology, 2018, Light Microscopy Method and Protocols, Markaki and Harz eds., Humana Press, New York, New York, ISBN-13:978-1493983056, which is hereby incorporated by reference.

In some embodiments, the second two-dimensional image is a bright-field microscopy image in which the imaged biological sample appears dark on a bright background. In some such embodiments, the biological sample has been stained. For instance, in some embodiments, the biological sample has been stained with Hematoxylin and Eosin and the second two-dimensional image is a bright-field microscopy image. In some embodiments the biological sample has been stained with a Periodic acid-Schiff reaction stain (stains carbohydrates and carbohydrate rich macromolecules a deep red color) and the second two-dimensional image is a bright-field microscopy image. In some embodiments the two-dimensional sample has been stained with a Masson's trichrome stain (nuclei and other basophilic structures are stained blue, cytoplasm, muscle, erythrocytes and keratin are stained bright-red, collagen is stained green or blue, depending on which variant of the technique is used) and the second two-dimensional image is a bright-field microscopy image. In some embodiments, the biological sample has been stained with an Alcian blue stain (a mucin stain that stains certain types of mucin blue, and stains cartilage blue and can be used with H&E, and with van Gieson stains) and the second two-dimensional image is a bright-field microscopy image. In some embodiments the two-dimensional sample has been stained with a van Gieson stain (stains collagen red, nuclei blue, and erythrocytes and cytoplasm yellow, and can be combined with an elastin stain that stains elastin blue/black) and the second two-dimensional image is a bright-field microscopy image. In some embodiments the biological sample has been stained with a reticulin stain, an Azan stain, a Giemsa stain, a Toluidine blue stain, an isamin blue/eosin stain, a Nissl and methylene blue stain, and/or a sudan black and osmium stain and the second two-dimensional image is a bright-field microscopy image.

In some embodiments, rather than being a bright-field microscopy image of a sample, the second two-dimensional image is an immunohistochemistry (IHC) image. IHC imaging may utilize a staining technique using antibody labels. One form of immunohistochemistry (IHC) imaging is immunofluorescence (IF) imaging. In an example of IF imaging, primary antibodies are used that specifically label a protein (here the non-nucleic analyte) in the biological sample, and then a fluorescently labelled secondary antibody or other form of probe is used to bind to the primary antibody, to show up where the first (primary) antibody has bound. A light microscope, equipped with fluorescence, is used to visualize the staining. The fluorescent label is excited at one wavelength of light and emits light at a different wavelength. Using the right combination of filters, the staining pattern produced by the emitted fluorescent light is observed. In some embodiments, a biological sample is exposed to several different primary antibodies (or other forms of probes) in order to quantify several different proteins in a biological sample. In some such embodiments, each such respective different primary antibody (or probe) is then visualized with a different fluorescence label (different channel) that fluoresces at a unique wavelength or wavelength range (relative to the other fluorescence labels used). In this way, several different proteins in the biological sample can be visualized.

In some embodiments of the present disclosure, in addition to bright-field imaging or instead of bright-field imaging, fluorescence imaging can be used to acquire the second two-dimensional image of the sample. As used herein the term “fluorescence imaging” refers to imaging that relies on the excitation and re-emission of light by fluorophores, regardless of whether they are added experimentally to the sample and bound to antibodies (or other compounds) or naturally occurring features of the sample. The above-described IHC imaging, and in particular IF imaging, is just one form of fluorescence imaging. Accordingly, in some embodiments, the second two-dimensional image of the biological sample represents a respective channel in one or more channels, where each respective channel in the one or more channels represents an independent (e.g., different) wavelength or a different wavelength range (e.g., corresponding to a different emission wavelength).

In some embodiments in which fluorescence imaging is conducted, the second two-dimensional image is acquired using Epi-illumination mode, where both the illumination and detection are performed from one side of the biological sample. In some such embodiments, the second two-dimensional image is acquired using confocal microscopy, two-photon imaging, wide-field multiphoton microscopy, single plane illumination microscopy or light sheet fluorescence microscopy. See, for example, Adaptive Optics for Biological Imaging, 2013, Kubby ed., CRC Press, Boca Raton, Florida; and Confocal and Two-Photon Microscopy: Foundations, Applications and Advances, 2002, Diaspro ed., Wiley Liss, New York, New York; and Handbook of Biological Confocal Microscopy, 2002, Pawley ed., Springer Science+Business Media, LLC, New York, New York each of which is hereby incorporated by reference.

In some embodiments, the second two-dimensional image is obtained using one or more immunohistochemistry (IHC) probes that excite at various different wavelengths. See, for example, Day and Davidson, 2014, “The Fluorescent Protein Revolution (In Cellular and Clinical Imaging),” CRC Press, Taylor & Francis Group, Boca Raton, Florida; “Quantitative Imaging in Cell Biology” Methods in Cell Biology 123, 2014, Wilson and Tran, eds.; Advanced Fluorescence Reporters in Chemistry and Biology II: Molecular Constructions, Polymers and Nanoparticles (Springer Series on Fluorescence), 2010, Demchenko, ed., Springer-Verlag, Berlin, Germany; Fluorescence Spectroscopy and Microscopy: Methods and Protocols (Methods in Molecular Biology) 2014th Edition, 2014, Engelborghs and Visser, eds., HumanPress, each of which is hereby incorporated by reference for their disclosure on fluorescence imaging.

In some embodiments, the first or second two-dimensional image is obtained in any electronic color mode, including but not limited to grayscale, bitmap, indexed, RGB, CMYK, HSV, lab color, duotone, and/or multichannel. In some embodiments, the first or second two-dimensional image is manipulated (e.g., stitched, compressed and/or flattened). In some embodiments, a respective two-dimensional image is a color image (e.g., 3×8 bit, 512×512 pixel resolution). In some embodiments, a respective two-dimensional image is a grey-scale image (e.g., 1×8 bit, 512×512 pixel resolution).

For instance, in some embodiments, the first two-dimensional image is a color image, and the second two-dimensional image is a grayscale image.

In some embodiments, a respective two-dimensional image (e.g., the second image) is obtained using a plurality of channels, each respective channel in the plurality of channels comprising a respective instance of the image acquired at a different respective illumination. In some such embodiments, each respective channel in the plurality of channels represents an independent (e.g., different) wavelength or a different wavelength range (e.g., corresponding to a different emission wavelength). In some embodiments, the plurality of channels includes at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 channels. In some embodiments, the plurality of channels includes no more than 40, no more than 20, no more than 15, no more than 10, no more than 8, or no more than 5 channels. In some embodiments, the plurality of channels comprises between 2 and 5 channels, between 2 and 10 channels, or between 3 and 15 channels. In some embodiments, the plurality of channels includes at least 3 channels corresponding to a red channel, a green channel, and a blue channel.

In some embodiments, a two-dimensional image (e.g., the second image) is a multichannel image comprising, for each respective channel in a plurality of channels, a respective instance of the image acquired at a different respective illumination, and the obtaining (e.g., receiving) the second image comprises using all of the instances of the image across the plurality of channels.

In some embodiments, a respective two-dimensional image (e.g., the second two-dimensional image) is a multichannel image comprising, for each respective channel in a plurality of channels, a respective instance of the image acquired at a different respective illumination, and the obtaining (e.g., receiving) the second two-dimensional image comprises selecting a respective channel for the two-dimensional image from the plurality of channels, thereby obtaining (e.g., receiving) the respective instance of the image corresponding to the respective channel.

In some embodiments, a respective two-dimensional image (e.g., the second two-dimensional image) is a multichannel two-dimensional image comprising, for each respective channel in a plurality of channels, a respective instance of the image acquired at a different respective illumination, and the obtaining (e.g., receiving) the second two-dimensional image comprises selecting a first respective channel for the image from the plurality of channels for a first process and selecting a second respective channel for the image from the plurality of channels for a second process, thereby obtaining (e.g., receiving) a first instance of the image for the first process and a second instance of the image for the second process. Thus, in some such implementations, different instances of a respective image obtained at different illuminations are utilized for different processes relating to image analysis, such as fiducial registration and tissue segmentation.

As an example, in some embodiments, the receiving a second two-dimensional image comprises selecting a channel for the second image from a plurality of channels, each respective channel in the plurality of channels comprising a respective instance of the second image acquired at a different respective illumination. In some embodiments, the plurality of channels comprises a first instance of the second image acquired at a first respective illumination that causes a contrast of the biological sample to be lower than a contrast of the one or more spatial fiducials of the second substrate, and a second instance of the second image acquired at a second respective illumination that causes a contrast of the biological sample to be higher than a contrast of the one or more spatial fiducials of the second substrate. Thus, in some embodiments, the first instance of the second image can be used to perform a detection of spatial fiducials, and the second instance of the second image can be used to perform a tissue segmentation to locate the biological sample in the image.

In some embodiments, the first illumination includes a wavelength between 564 nm and 580 nm or a wavelength between 700 nm and 1 mm. In some embodiments, the second illumination includes a wavelength between 534 nm and 545 nm.

Referring to block 234 of FIG. 2C, in some embodiments there is removed, from the first subset of the first plurality of pixels, clusters of pixels within the first image that constitute less than a threshold number of pixels. Further there is removed, from the first subset of the second plurality of pixels, clusters of pixels within the second image that constitute less than a threshold number of pixels. Referring to block 236 of FIG. 2C, in some such embodiments, the threshold number of pixels is 10, 11, 12, 13, 14, 15, 16, 17, 18, 19, 20, 21, 22, 23, 24, 25, 26, 27, 28, 29, or 30 pixels. That is, each pixel that is not part of a contiguous cluster of pixels having this minimum number of pixels is removed from the image. Removal of a respective pixel from an image in this context means resetting the pixel value of the respective pixel to zero or some other nominal value that will indicate that the pixel value is not to be used in image comparisons. The purpose of such pixel removal is to remove small contaminants or artifacts in the two-dimensional images.

In some embodiments, block 234 is performed after the thresholding of blocks 260 and 262 described below.

Referring to block 240 of FIG. 2D, in some embodiments the first subset of the first plurality of pixels occupies a first plurality of disjoint areas within the first image. The first subset of the second plurality of pixels occupies a second plurality of disjoint areas within the second image. Referring to block 242 of FIG. 2D, in some such embodiments the first plurality of disjoint areas comprises five disjoint areas and the second plurality of disjoint areas comprises five disjoint areas. In some embodiments, each such disjoint area represents a cluster of contiguous pixels that satisfies the threshold requirement for a minimum number of contiguous pixels references above with respect to block 234. Each such disjoint area in the first two-dimensional image represents measured abundance of the nucleic acid analyte in a different portion of the biological sample. Each such disjoint area in the second two-dimensional image represents measured abundance of the non-nucleic acid analyte in a different portion of the biological sample. In some embodiments the first plurality of disjoint areas is two or more, three or more, four or more, five or more, six or more, seven or more, eight or more, nine or more, or ten or more disjoint areas. In some embodiments the first plurality of disjoint areas is between five and two hundred disjoint areas. In some embodiments the first plurality of disjoint areas is between five and 5,000 disjoint areas. In some embodiments the second plurality of disjoint areas is two or more, three or more, four or more, five or more, six or more, seven or more, eight or more, nine or more, or ten or more disjoint areas. In some embodiments the second plurality of disjoint areas is between five and two hundred disjoint areas. In some embodiments the second plurality of disjoint areas is between five and 5,000 disjoint areas. In some embodiments, each disjoint area in the first and second plurality of disjoint areas satisfies a threshold requirement described in block 234. In some embodiments, there are more disjoint areas in the first plurality of disjoint areas than in the second plurality of disjoint areas. In some embodiments, there are more disjoint areas in the second plurality of disjoint areas than in the first plurality of disjoint areas. In some embodiments, the number of disjoint areas in the first plurality of disjoint areas and the second plurality of disjoint areas is the same.

Referring to block 244 of FIG. 2D, in some embodiments the intensity of the first plurality of pixels and the second plurality of pixels is on a log₂ based scale relative to measured intensity. In some such embodiments, the method comprises performing a normalization of pixel values within the first image and/or the second image. In some embodiments, the normalization is a log normalization. In some such embodiments, the performing the normalization comprises, for each respective pixel in the first image, reassigning the pixel value to the log of the pixel value when the respective pixel has a corresponding pixel value that is greater than a threshold value, such as 1, and performing a linear transformation across the plurality of pixels in the first image, such that the pixel value of each respective pixel in the first image is normalized to a corresponding value within a particular range, such as between 0 and 1. In some embodiments, the threshold value is a value other than 1, such as a number between 0.5 and 100, and such that the pixel value of each respective pixel in the first image is normalized to a corresponding value within the range 0 to this number (between 0.5 and 100). For example, in some embodiments, for each respective pixel in the first image, the pixel value is reassigned to the log of the pixel value when the respective pixel has a corresponding pixel value that is greater than 10, and a linear transformation is performed across the plurality of pixels in the first image, such that the pixel value of each respective pixel in the first image is normalized to a corresponding value within the particular range 0 and 10.

In some such embodiments, the performing the normalization comprises, for each respective pixel in the second image, reassigning the pixel value to the log of the pixel value when the respective pixel has a corresponding pixel value that is greater than 1, and performing a linear transformation across the plurality of pixels in the second image, such that the pixel value of each respective pixel in the second image is normalized to a corresponding value between 0 and 1. In some embodiments, the threshold value is a value other than 1, such as a number between 0.5 and 100, and such that the pixel value of each respective pixel in the second image is normalized to a corresponding value within the range 0 to this number (between 0.5 and 100). For example, in some embodiments, for each respective pixel in the second image, the pixel value is reassigned to the log of the pixel value when the respective pixel has a corresponding pixel value that is greater than 10, and a linear transformation is performed across the plurality of pixels in the second image, such that the pixel value of each respective pixel in the second image is normalized to a corresponding value within the particular range 0 and 10.

Other suitable methods of image normalization and modification are contemplated, including smoothing, noise reduction, color normalization, contrast stretching, histogram stretching, Reinhard method, Macenko method, stain color descriptor (SCD), complete color normalization and structure preserving color normalization (SPCN), as will be apparent to one skilled in the art. See, e.g., Roy et al., “Novel Color Normalization Method for Hematoxylin & Eosin Stained Histopathology Images,” 2019 IEEE Access 7:2169-3536; doi: 10.1109/ACCESS.2019.2894791, which is hereby incorporated herein by reference in its entirety.

Referring to block 246 of FIG. 2D, in some embodiments the first subset of the second plurality of pixels comprises 100, 200, 300, 400, 500, or 1000 pixels and a registration between the first and second image is determined. In some embodiments the first subset of the second plurality of pixels consists of between 50 and 5000 pixels and a registration between the first and second image is determined. In some embodiments, the first and second image are the same size (e.g., same number of pixels in a two-dimensional array). In some such embodiments, the registration between the first and second image is based on the fact that that the two images are the same size. Thus, for example, considering the first image as a first two-dimensional array of pixels and the second image as a second two-dimensional array of pixels, a pixel at coordinates x, y in the first image corresponds to a pixel at coordinates x, y in the second image. In some embodiments, the registration imposes a geometric transformation or local displacement of one of the first and second image onto the other of the first and second image. In some embodiments, the geometric transformation is a rigid transformation. A rigid transformation allows only for translation and rotation. Thus, when the registration between the first and second images is a rigid transformation, the one of the first and second images is rotated and/or translated onto the other of the first and second images in accordance with the transformation. In some embodiments, the transformation is a non-rigid transform that comprises anisotropic scaling and skewing of one of the first and second images to the other of the first and second images. In some embodiments the non-rigid transform is an affine transformation.

In some embodiments, the transformation transforms a first plurality of pixel coordinates for the first image relative to a second plurality of pixel coordinates for the second image, using any of the transformation methods disclosed herein. In some embodiments, the transformation transforms a second plurality of pixel coordinates for the second image relative to a first plurality of pixel coordinates for the first image, using any of the transformation methods disclosed herein.

In some embodiments, the transformation is a two-dimensional similarity transform. In some embodiments, the transformation is selected from the group consisting of: affine transform, azimuth elevation to cartesian transform, BSpline deformable transform, centered affine transform, centered Euler 3D transform, centered rigid 2D transform, centered similarity 2D transform, elastic body reciprocal spline kernel transform, elastic body spline kernel transform, Euler 2D transform, Euler 3D transform, fixed center of rotation affine transform, identity transform, kernel transform, matrix offset transform, quaternion rigid transform, rigid 2D transform, rigid 3D perspective transform, rigid 3D transform, scalable affine transform, scale logarithmic transform, scale skew versor 3D transform, scale transform, scale versor 3D transform, similarity 2D transform, similarity 3D transform, thin plate R2 Log R spline kernel transform, thin plate spline kernel transform, transform, transform base, translation transform, versor rigid 3D transform, versor transform, and volume spline kernel transform.

In some embodiments the alignment algorithm used to determine the registration between the first and second images is a coherent point drift algorithm. See, Myronenko et al., 2007, “Non-rigid point set registration: Coherent Point Drift,” NIPS, 1009-1016; and Myronenko and Song, “Point Set Registration: Coherent Point Drift,” arXiv:0905.2635v1, 15 May 2009, each of which is hereby incorporated by reference, for disclosure on the coherent point drift algorithm. In some embodiments, the coherent point drift algorithm that is used is an implementation in Python called pycpd. See, the Internet at github.com/siavashk/pycpd, which is hereby incorporated by reference.

In some embodiments the alignment algorithm that is used to determine the registration between the first and second images is an iterative closest point algorithm. See, for example, Chetverikov et al., 2002, “the Trimmed Iterative Closest Point Algorithm,” Object recognition supported by user interaction for service robots, Quebec City, Quebec, Canada, ISSN: 1051-4651; and Chetverikov et al., 2005, “Robust Euclidean alignment of 3D point sets; the trimmed iterative closest point algorithm,” Image and Vision Computing 23(3), pp. 299-309, each of which is hereby incorporated by reference.

In some embodiments the alignment algorithm used to determine the registration between the first and second images is a robust point matching algorithm (See, for example, Chui and Rangarajanb, 2003, “A new point matching algorithm for non-rigid registration,” Computer Vision and Image Understanding 89(2-3), pp. 114-141, which is hereby incorporated by reference) or a thin-plate-spline robust point matching algorithm (See, for example, Yang, 2011, “The thin plate spline robust point matching (TPS-RPM) algorithm: A revisit,” Pattern Recognition Letters 32(7), pp. 910-918, which is hereby incorporated by reference.)

Referring to block 248 of FIG. 2D, in some embodiments the first image 36 is displayed on a display in electronic communication with the computer system. User selection of a first region of the first image is obtained, where each pixel in the first subset of pixels in the first plurality of pixels is within the first region. For instance, referring to FIG. 7, a user can use Lasso tool 704 or some other selection tool to select the pixels 42 in region 702 that represent the UMI counts of the nucleic acid analyte. Each respective pixel 42 outside the first region (e.g., region 702 of FIG. 7) is removed from the first image 36 (e.g., the pixel intensity value 46 for the respective pixel 42 is set to zero) prior to obtaining the threshold value of the first image.

Referring to block 250 of FIG. 2D, in some such embodiments, a second region 802 is defined in the second image 48 as illustrated in FIG. 8. The second region 802 includes a corresponding pixel 54 for each pixel in the first subset of the second plurality of pixels of the second image. In some embodiments, each pixel 54 in the second region 802 in the second image 48 corresponds to a pixel 42 in the first region 702 of the first image 36 in accordance with the registration. Each pixel 54 in the first subset of pixels in the second plurality of pixels is within the second region 802. Further, each respective pixel 54 outside the second region 802 is removed from the second image 48 (e.g. the pixel intensity value 58 of the respective pixel is set to a value of zero) prior to obtaining the threshold value of the second image 48.

Referring to block 254 of FIG. 2E, in some embodiments, the nucleic acid analyte is a plurality of RNA or DNA molecules, arising from the biological sample, where each RNA or DNA molecule in the plurality of RNA or DNA molecules encodes all or a portion of a first gene, and the spatial analyte UMI abundance data for the nucleic acid analyte quantifies UMI abundance of a plurality of spatially barcoded sequence reads of the plurality of RNA or DNA molecules. Referring to block 256, in some embodiments the non-nucleic acid analyte is a first protein and the spatial analyte abundance data for the non-nucleic acid analyte quantifies abundance of a labeled antibody to the first protein. Referring to block 258, in some embodiments the first gene encodes the first protein.

In some embodiments, to extract the signal from the biological sample, the first and second images are transformed from RGB (red, green, blue) image space to HSV (hue saturation value) image space. In some embodiments the hue of each respective pixel within the HSV image space is used as the pixel intensity value for the respective pixel although the present disclosure is not so limited. In some embodiments, any pixel intensity value derived from RGB or HSV values, or subsets thereof, can be used to store the intensity value for a pixel in accordance with the present disclosure.

Referring to block 260 of FIG. 2E, a first threshold value of the first image is obtained. Referring to block 262 of FIG. 2E, a second threshold value of the second image is obtained. Referring to block 264 of FIG. 2E, in some such embodiments the first image is segmented to identify the first subset of the first plurality of pixels using a first segmentation algorithm and the second image is segmented to identify the first subset of the second plurality of pixels using a second segmentation algorithm. In some embodiments, only those pixels that were retained after performance of block 234 and/or block 248 and/or block 250 are used in this thresholding.

Referring to block 266 of FIG. 2E, in some such embodiments, the first segmentation algorithm and the second segmentation algorithm is a binarization method using global thresholding. See for example, Chacki et al, 2014, “A Comprehensive Survey on Image Binarization Techniques,” in Studies in Computational Intelligence 560, DOI: 10.1007/978-81-322-1907-1_2, which is hereby incorporated by reference.

Referring to block 270 of FIG. 2F, in some embodiments the first segmentation algorithm identifies the first threshold value as a first pixel intensity value that that divides the pixels of the first image into either the first subset of the first plurality of pixels or a second subset of the first plurality of pixels, where the first threshold value represents a minimization of intra-class intensity variance between the first and second subset of the first plurality of pixels or a maximization of inter-class variance between the first and second subset of the first plurality of pixels. See, for example, Bangare et al., 2015, “Reviewing Otsu's Method For Image Thresholding,” International Journal of Applied Engineering Research 10(9), pp. 21777-21783, which is hereby incorporated by reference.

In some embodiments, the first segmentation algorithm and the second segmentation algorithm is a Graph cut segmentation algorithm. Graph cut is an optimization-based binarization technique which uses polynomial-order computations to achieve robust segmentation even when foreground and background pixel intensities are poorly segregated. See, Rother et al., 2004, “‘GrabCut’—Interactive Foreground Extraction using Iterated Graph Cuts,” ACM Transactions on Graphics. 23 (3): 309-314, doi:10.1145/1186562.1015720, which is hereby incorporated herein by reference in its entirety. See also, Boykov and Jolly, 2001, “Interactive graph cuts for optimal boundary and region segmentation of objects in N-D images,” Proc. IEEE Int. Conf. on Computer Vision, CD-ROM, and Greig et al., 1989, “Exact MAP estimation for binary images,” J. Roy. Stat. Soc. B. 51, 271-279, for details on graph cut segmentation algorithms; and Chuang et al., 2001, “A Bayesian approach to digital matting,” Proc. IEEE Conf. Computer Vision and Pattern Recog., CD-ROM, for details on Bayes matting models and alpha-mattes, each of which is hereby incorporated herein by reference in its entirety. In some embodiments, the graph cut segmentation algorithm is a GrabCut segmentation algorithm.

In some embodiments, the first segmentation algorithm and the second segmentation algorithm is a Graph cut segmentation algorithm implemented in accordance with Tao et al., 2008, “Image Thresholding Using Graph Cuts,” IEEE Transactions on Systems, Man, and Cybernetics—Part A: Systems and Humans 38(5), pp. 1181-1195, which is hereby incorporated by reference.

Referring to block 272 of FIG. 2F, there is determined, using the registration 59 between the first and second images, a first summation of the respective UMI abundance value of each respective pixel in the first subset of the first plurality of pixels in which the corresponding pixel in the first subset of the second plurality of pixels exceeds the second threshold value.

That is, the first summation 60 is summation of the pixels 42 in the first two-dimensional image 36 having pixel intensity values 46 that are above the first image threshold value 40 that are in regions where there are pixel values 58 in corresponding regions in the second image 48 that are above the second image threshold value 52 of the second image. In some embodiments, the first summation represents the nonzero pixels of the first image where there are also nonzero pixels in corresponding pixels in the second image (e.g., mutual signal overlap between the first image and the second image).

In some embodiments, the registration between the first image and the second image is determined by downsampling the first image and/or the second image so that they are the same size. In some embodiments, the registration between the first image and the second image is determined by virtue of the fact that the first image and the second image are the same size.

Referring to block 274 of FIG. 2F, in some embodiments the first image is downsampled to the size of the second image prior to determining, using the registration, the first summation. In some such embodiments, the method comprises downsampling the first image (e.g., where the first image is a high resolution image, and the second image is a low resolution image). In some embodiments, the downsampling the first image comprises downsampling the first image to a resolution that is no lower than the resolution of the second image. In some embodiments, the downsampling the first image comprises downsampling the first image to a resolution that is no lower than 2× the resolution of the second image. In some embodiments, the downsampling the first image comprises downsampling the first image to a resolution that is no lower than 1×, no lower than 1.5×, no lower than 2×, no lower than 3×, no lower than 4×, no lower than 5×, no lower than 6×, no lower than 7×, no lower than 8×, no lower than 9×, or no lower than 10× the resolution of the second image. In some embodiments, the downsampling the first image comprises downsampling the first image to a resolution that is no higher than 20×, no higher than 15×, no higher than 10×, or no higher than 5× the resolution of the second image. In some embodiments, the downsampling the first image comprises downsampling the first image to a resolution that is from 1× to 5×, from 2× to 10×, or from 1.5× to 20× the resolution of the second image. In some embodiments, the downsampling the first image comprises downsampling the first image to a resolution that falls within another range starting no lower than 1× the resolution of the second image and ending no higher than 20× the resolution of the second image.

Referring to block 276 of FIG. 2F, in some embodiments the second image is downsampled to a size of the first image prior to determining, using the registration, the first summation. In some such embodiments, the method comprises downsampling the second image (e.g., where the second image is a high resolution image, and the first image is a low resolution image). In some embodiments, the downsampling the second image comprises downsampling the second image to a resolution that is no lower than the resolution of the first image. In some embodiments, the downsampling the second image comprises downsampling the second image to a resolution that is no lower than 2× the resolution of the first image. In some embodiments, the downsampling the second image comprises downsampling the second image to a resolution that is no lower than 1×, no lower than 1.5×, no lower than 2×, no lower than 3×, no lower than 4×, no lower than 5×, no lower than 6×, no lower than 7×, no lower than 8×, no lower than 9×, or no lower than 10× the resolution of the first image. In some embodiments, the downsampling the second image comprises downsampling the second image to a resolution that is no higher than 20×, no higher than 15×, no higher than 10×, or no higher than 5× the resolution of the first image. In some embodiments, the downsampling the second image comprises downsampling the second image to a resolution that is from 1× to 5×, from 2× to 10×, or from 1.5× to 20× the resolution of the first image. In some embodiments, the downsampling the second image comprises downsampling the second image to a resolution that falls within another range starting no lower than 1× the resolution of the first image and ending no higher than 20× the resolution of the first image.

Referring to block 278 of FIG. 2G, the first summation, normalized to a second summation of each respective UMI abundance value of each pixel in the first subset of the first plurality of pixels, is provided as a first measure of colocalization between (i) the spatial analyte UMI abundance data for the nucleic acid analyte and (ii) the spatial analyte abundance data for the non-nucleic acid analyte.

In some embodiments in accordance with block 278, the normalized first summation has the form:

$${M}_{1} = \frac{{\sum }_{i} {R}_{i} , colocal}{{\sum }_{i} {R}_{i}}$$

where,
M₁ (Mander's Overlap Coefficient) is the first summation, normalized to the second summation,
Σᵢ Rᵢ, colocal is the first summation—the summation of each respective UMI abundance value of each pixel in the first subset of the first plurality of pixels (of the first image) in which the corresponding pixel in the first subset of the second plurality of pixels (of the second image) exceeds the second threshold value,
Σᵢ Rᵢ is the second summation—the summation of each respective UMI abundance value of each pixel in the first subset of the first plurality of pixels (of the first image), and
i is an index to each pixel in the first subset of the first plurality of pixels.

In some embodiments, each respective pixel in the second plurality of pixels in the second image corresponds to a respective pixel in the first plurality of pixels in the first image. In some embodiments, each respective pixel in the second plurality of pixels in the second image corresponds to multiple pixels in the first plurality of pixels in the first image. In some embodiments, each respective pixel in the first plurality of pixels in the first image corresponds to a respective pixel in the corresponding pixels in the second plurality of pixels in the second image. In some embodiments, each respective pixel in the first plurality of pixels in the first image corresponds to multiple pixels in the corresponding pixels in the second plurality of pixels in the second image. Accordingly, in some embodiments, the pixels in a respective image (e.g., the first image and/or the second image) used for determining the normalized first summation comprises at least 0.005%, at least 0.008%, at least 0.01%, at least 0.02%, at least 0.03%, at least 0.04%, at least 0.05%, at least 0.06%, at least 0.07%, at least 0.08%, at least 0.09%, at least 0.1%, at least 0.2%, at least 0.3%, at least 0.4%, at least 0.5%, at least 0.6%, at least 0.7%, at least 0.8%, at least 0.9%, at least 1%, at least 2%, at least 3%, at least 4%, at least 5%, at least 6%, at least 7%, at least 8%, at least 9%, at least 10%, at least 15%, at least 20%, at least 30%, or at least 50% of the total pixels in the respective image. In some embodiments, the plurality of pixels in a respective image (e.g., the first image and/or the second image) used for determining the normalized first summation comprises no more than 70%, no more than 50%, no more than 30%, no more than 20%, no more than 10%, no more than 5%, no more than 1%, no more than 0.5%, or no more than 0.1% of the total pixels in the respective image. In some embodiments, the plurality of pixels in a respective image (e.g., the first image and/or the second image) used for determining the normalized first summation comprises from 0.01% to 10%, from 0.1% to 20%, from 0.05% to 1%, from 0.005% to 30%, from 0.5% to 15%, or from 1% to 10% of the total pixels in the respective image.

Referring to block 280 of FIG. 2G, in some embodiments a Pearson's correlation coefficient is also determined using (i) the intensity of each pixel in the first subset of the first plurality of pixels, (ii) the intensity of each pixel in the second image corresponding to a pixel in the first subset of the first plurality of pixels, (iii) a first mean value across the first subset of the first plurality of pixels, and (iv) a second mean value across the pixels in the second image corresponding to the first subset of pixels in the first plurality of pixels. The Pearson's correlation coefficient is then provided as a third measure of colocalization between (i) the spatial analyte UMI abundance data for the nucleic acid analyte and (ii) the spatial analyte abundance data for the non-nucleic acid analyte. In some embodiments, the Pearson's correlation coefficient has the form:

$$PCC = \frac{{\sum }_{i} ( {R}_{i} - \overline{R} ) \times ( {G}_{i} - \overline{G} )}{\sqrt{{\sum }_{i} {( {R}_{i} - \overline{R} )}^{2} \times {( {G}_{i} - \overline{G} )}^{2}}}$$

where
Σᵢ is a summation across all pixels in the first subset of the first plurality of pixels in the first image,
i is an index to each pixel in the first subset of the first plurality of pixels in the first image,
Rᵢ is the UMI count of the nucleic acid analyte at pixel i in the first image,
Gᵢ is an abundance of the non-nucleic analyte (in the form of a UMI count or a count of a marker associated with the non-nucleic analyte) at a pixel in second image that corresponds to pixel i in the first image,
R̅ is a mean of the UMI count of the nucleic acid analyte across all pixels i in the first subset of the first plurality of pixels in the first image, and
G̅ is a mean count of the of the non-nucleic analyte (in the form of a UMI count or a count of a marker associated with the non-nucleic analyte) across all pixels in the second image that correspond to a pixel in the first subset of the first plurality of pixels in the first image.

Referring to block 282 of FIG. 2G, in some embodiments a ratio is determined between a number of pixels in the first subset of the first plurality of pixels and a number of pixels in the first subset of the second plurality of pixels. The ratio is then provided as an indication of a size comparison between (i) the spatial analyte UMI abundance data for the nucleic acid analyte and (ii) the spatial analyte abundance data for the non-nucleic acid analyte.

Referring to block 284 of FIG. 2G, in some embodiments there is determined, using the registration, a third summation of the respective UMI abundance value of each respective pixel in the first subset of the second plurality of pixels in which the corresponding pixel in the first subset of the first plurality of pixels exceeds the first threshold value. The third summation, normalized to a fourth summation of each respective abundance value of each pixel in the first subset of the second plurality of pixels, is then provided as a second measure of colocalization between (i) the spatial analyte UMI abundance data for the nucleic acid analyte and (ii) the spatial analyte abundance data for the non-nucleic acid analyte.

In some embodiments in accordance with block 284, the third summation normalized to the fourth summation has the form:

$${M}_{2} = \frac{{\sum }_{i} {G}_{i} , colocal}{{\sum }_{i} {G}_{i}}$$

where,
M₂ (Mander's Overlap Coefficient) is the third summation,
Σᵢ Gᵢ, colocal is the third summation—the summation of each respective UMI abundance value of each pixel in the first subset of the second plurality of pixels (of the second image) in which the corresponding pixel in the first subset of the first plurality of pixels (of the first image) exceeds the first threshold value,
Σᵢ G is the fourth summation—the summation of each respective UMI abundance value (or other form of abundance value) of each pixel in the first subset of the second plurality of pixels (of the second image), and
i is an index to each pixel in the first subset of the second plurality of pixels.

Referring to block 286 of FIG. 2G, in some embodiments the first measure of colocalization is used to characterize a biological condition of the subject. In some embodiments, the first measure of colocalization and the second measure of colocalization is used to characterize a biological condition of the subject. In some embodiments, the first measure of colocalization, the second measure of colocalization, the ratio between the number of pixels in the first subset of the first plurality of pixels and the number of pixels in the first subset of the second plurality of pixels is used to characterize a biological condition of the subject. In some embodiments, the first measure of colocalization of block 278, the second measure of colocalization of block 284, the ratio between the number of pixels in the first subset of the first plurality of pixels and the number of pixels in the first subset of the second plurality of pixels of block 282, and the Pearson's correlation coefficient of block 280 is used to characterize a biological condition in a subject. In some embodiments, the first measure of colocalization of block 278 and any combination of (i) the second measure of colocalization of block 284, (ii) the ratio between the number of pixels in the first subset of the first plurality of pixels and the number of pixels in the first subset of the second plurality of pixels of block 282, and (iii) the Pearson's correlation coefficient of block 280 is used to characterize a biological condition in a subject.

In some embodiments, the biological condition is absence or presence of a disease. In some embodiments, the biological condition is a stage of a disease. In some embodiments the biological condition is a neurological condition (e.g., autism spectrum disorder, schizophrenia, or attention-deficit/hyperactivity disorder (ADHD), neurodegenerative condition (e.g., amyotrophic lateral sclerosis (ALS), Alzheimer's disease, Parkinson's disease, and Huntington's disease), or a cancer (e.g., pediatric cancer). In some embodiments, the biological condition is absence or presence of a cancer such as a carcinoma, lymphoma, blastoma, sarcoma, and leukemia or lymphoid malignancy. More particular examples of such cancers include squamous cell cancer (e.g., epithelial squamous cell cancer), lung cancer including small-cell lung cancer, non-small cell lung cancer (“NSCLC”), adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, as well as head and neck cancer.

In some embodiments the first measure of colocalization provides an extent to which (i) the spatial analyte UMI abundance data for the nucleic acid analyte and (ii) the spatial analyte abundance data for the non-nucleic acid analyte are colocalized in the biological sample. In some embodiments, the first measure of colocalization and the second measure of colocalization provides an extent to which (i) the spatial analyte UMI abundance data for the nucleic acid analyte and (ii) the spatial analyte abundance data for the non-nucleic acid analyte are colocalized in the biological sample. In some embodiments, the first measure of colocalization, the second measure of colocalization, the ratio between the number of pixels in the first subset of the first plurality of pixels and the number of pixels in the first subset of the second plurality of pixels provides an extent to which (i) the spatial analyte UMI abundance data for the nucleic acid analyte and (ii) the spatial analyte abundance data for the non-nucleic acid analyte are colocalized in the biological sample. In some embodiments, the first measure of colocalization of block 278, the second measure of colocalization of block 284, the ratio between the number of pixels in the first subset of the first plurality of pixels and the number of pixels in the first subset of the second plurality of pixels of block 282, and the Pearson's correlation coefficient of block 280 provides an extent to which (i) the spatial analyte UMI abundance data for the nucleic acid analyte and (ii) the spatial analyte abundance data for the non-nucleic acid analyte are colocalized in the biological sample. In some embodiments, the first measure of colocalization of block 278 and any combination of (i) the second measure of colocalization of block 284, (ii) the ratio between the number of pixels in the first subset of the first plurality of pixels and the number of pixels in the first subset of the second plurality of pixels of block 282, and (iii) the Pearson's correlation coefficient of block 280 provides an extent to which (i) the spatial analyte UMI abundance data for the nucleic acid analyte and (ii) the spatial analyte abundance data for the non-nucleic acid analyte are colocalized in the biological sample.

In some embodiments the first measure of colocalization provides an extent to which (i) the spatial analyte UMI abundance data for the nucleic acid analyte and (ii) the spatial analyte abundance data for the non-nucleic acid analyte colocalize to a common component of a plurality of cells within the biological sample. In some embodiments, the first measure of colocalization and the second measure of colocalization provides an extent to which (i) the spatial analyte UMI abundance data for the nucleic acid analyte and (ii) the spatial analyte abundance data for the non-nucleic acid analyte colocalize to a common component of a plurality of cells within the biological sample. In some embodiments, the first measure of colocalization, the second measure of colocalization, the ratio between the number of pixels in the first subset of the first plurality of pixels and the number of pixels in the first subset of the second plurality of pixels provides an extent to which (i) the spatial analyte UMI abundance data for the nucleic acid analyte and (ii) the spatial analyte abundance data for the non-nucleic acid analyte colocalize to a common component of a plurality of cells within the biological sample. In some embodiments, the first measure of colocalization of block 278, the second measure of colocalization of block 284, the ratio between the number of pixels in the first subset of the first plurality of pixels and the number of pixels in the first subset of the second plurality of pixels of block 282, and the Pearson's correlation coefficient of block 280 provides an extent to which (i) the spatial analyte UMI abundance data for the nucleic acid analyte and (ii) the spatial analyte abundance data for the non-nucleic acid analyte colocalize to a common component of a plurality of cells within the biological sample. In some embodiments, the first measure of colocalization of block 278 and any combination of (i) the second measure of colocalization of block 284, (ii) the ratio between the number of pixels in the first subset of the first plurality of pixels and the number of pixels in the first subset of the second plurality of pixels of block 282, and (iii) the Pearson's correlation coefficient of block 280 provides an extent to which (i) the spatial analyte UMI abundance data for the nucleic acid analyte and (ii) the spatial analyte abundance data for the non-nucleic acid analyte colocalize to a common component of a plurality of cells within the biological sample. In any of the forgoing embodiments, the common component is a cell membrane, cytoplasm, a nucleus, a nuclear envelope, a nucleolus, an endoplasmic reticulum, a ribosome, a Golgi apparatus, a mitochondria, a lysosome, a vacuole, a peroxisome, a cytoskeleton, and/or a centriole.

The colocalization of gene expression with protein abundance in any of the forgoing embodiments is used in some embodiments to determine the precise cellular locations where specific proteins are synthesized. Such information is then used to further understanding of the roles and functions of proteins within different cellular compartments.

The extent to which (i) the spatial analyte UMI abundance data for the nucleic acid analyte and (ii) the spatial analyte abundance data for the non-nucleic acid analyte colocalize to a common component in any of the forgoing embodiments can be used, for example, to identify a function of a protein, for instance when a protein is found to colocalize with a marker of a particular organelle. In some such embodiments the non-nucleic acid analyte is the protein and the nucleic acid analyte is known to concentrate in the particular organelle.

The extent to which (i) the spatial analyte UMI abundance data for the nucleic acid analyte and (ii) the spatial analyte abundance data for the non-nucleic acid analyte colocalize to a common component in any of the forgoing embodiments can be used, as another example, to associate a protein with a particular biological pathway, for instance when the protein is found to colocalize with a marker of a particular pathway. In some such embodiments the non-nucleic acid analyte is the protein and the nucleic acid analyte is known to be in the particular pathway.

In some embodiments the first measure of colocalization is used to validate gene expression data. In some embodiments, the first measure of colocalization and the second measure of colocalization is used to validate gene expression data. In some embodiments, the first measure of colocalization, the second measure of colocalization, the ratio between the number of pixels in the first subset of the first plurality of pixels and the number of pixels in the first subset of the second plurality of pixels is used to validate gene expression data. In some embodiments, the first measure of colocalization of block 278, the second measure of colocalization of block 284, the ratio between the number of pixels in the first subset of the first plurality of pixels and the number of pixels in the first subset of the second plurality of pixels of block 282, and the Pearson's correlation coefficient of block 280 is used to validate gene expression data. In some embodiments, the first measure of colocalization of block 278 and any combination of (i) the second measure of colocalization of block 284, (ii) the ratio between the number of pixels in the first subset of the first plurality of pixels and the number of pixels in the first subset of the second plurality of pixels of block 282, and (iii) the Pearson's correlation coefficient of block 280 is used to validate gene expression data. For instance, such colocalization analysis, where the nucleic acid analyte is for a gene and the non-nucleic acid analyte is for a protein product of the gene, helps validate the accuracy of gene expression data obtained through techniques like RNA sequencing. For instance, such colocalization in accordance with the present disclosure is used in some embodiments to confirm that a gene is expressed at the mRNA level is also being translated into a functional protein. This colocalization allows for the verification that the protein corresponding to the mRNA is indeed present in the same cellular location.

In some embodiments the first measure of colocalization is used to elucidate a regulatory process that controls gene expression. In some embodiments, the first measure of colocalization and the second measure of colocalization is used to elucidate a regulatory process that controls gene expression. In some embodiments, the first measure of colocalization, the second measure of colocalization, the ratio between the number of pixels in the first subset of the first plurality of pixels and the number of pixels in the first subset of the second plurality of pixels is used to elucidate a regulatory process that controls gene expression. In some embodiments, the first measure of colocalization of block 278, the second measure of colocalization of block 284, the ratio between the number of pixels in the first subset of the first plurality of pixels and the number of pixels in the first subset of the second plurality of pixels of block 282, and the Pearson's correlation coefficient of block 280 is used to elucidate a regulatory process that controls gene expression. In some embodiments, the first measure of colocalization of block 278 and any combination of (i) the second measure of colocalization of block 284, (ii) the ratio between the number of pixels in the first subset of the first plurality of pixels and the number of pixels in the first subset of the second plurality of pixels of block 282, and (iii) the Pearson's correlation coefficient of block 280 is used to elucidate a regulatory process that controls gene expression. For instance, in such colocalization analysis, the nucleic acid analyte is for the gene and the non-nucleic acid analyte is for a possible regulatory element of the gene. For instance, in some embodiments such colocalization in accordance with the present disclosure is to confirm that a gene is regulated by the regulatory element. In some embodiments, failure to see such colocalization in accordance with the present disclosure is used to confirm that possible regulatory element does not regulate expression of the gene. In some embodiments the regulatory element is an RNA-binding protein.

In some embodiments the first measure of colocalization is used to elucidate whether gene expression and protein abundance patterns are altered in diseased cells or tissues. In some embodiments, the first measure of colocalization and the second measure of colocalization is used to elucidate whether gene expression and protein abundance patterns are altered in diseased cells or tissues. In some embodiments, the first measure of colocalization, the second measure of colocalization, the ratio between the number of pixels in the first subset of the first plurality of pixels and the number of pixels in the first subset of the second plurality of pixels is used to elucidate whether gene expression and protein abundance patterns are altered in diseased cells or tissues. In some embodiments, the first measure of colocalization of block 278, the second measure of colocalization of block 284, the ratio between the number of pixels in the first subset of the first plurality of pixels and the number of pixels in the first subset of the second plurality of pixels of block 282, and the Pearson's correlation coefficient of block 280 is used to elucidate whether gene expression and protein abundance patterns are altered in diseased cells or tissues. In some embodiments, the first measure of colocalization of block 278 and any combination of (i) the second measure of colocalization of block 284, (ii) the ratio between the number of pixels in the first subset of the first plurality of pixels and the number of pixels in the first subset of the second plurality of pixels of block 282, and (iii) the Pearson's correlation coefficient of block 280 is used to elucidate whether gene expression and protein abundance patterns are altered in diseased cells or tissues. For instance, in such colocalization analysis, the nucleic acid analyte is for the gene and the non-nucleic acid analyte is for a protein gene product (e.g., of the gene or another gene). In some such embodiments such colocalization studies provide context for disease research by helping identify whether gene expression and protein abundance patterns are altered in diseased cells or tissues. Additionally, in some embodiments the identification of colocalized genes and proteins is used to discover new biomarkers for various diseases.

In some embodiments the first measure of colocalization is used to determine stability and turnover rates of proteins. In some embodiments, the first measure of colocalization and the second measure of colocalization is used to determine stability and turnover rates of proteins. In some embodiments, the first measure of colocalization, the second measure of colocalization, the ratio between the number of pixels in the first subset of the first plurality of pixels and the number of pixels in the first subset of the second plurality of pixels is used to determine stability and turnover rates of proteins. In some embodiments, the first measure of colocalization of block 278, the second measure of colocalization of block 284, the ratio between the number of pixels in the first subset of the first plurality of pixels and the number of pixels in the first subset of the second plurality of pixels of block 282, and the Pearson's correlation coefficient of block 280 is used to determine stability and turnover rates of proteins. In some embodiments, the first measure of colocalization of block 278 and any combination of (i) the second measure of colocalization of block 284, (ii) the ratio between the number of pixels in the first subset of the first plurality of pixels and the number of pixels in the first subset of the second plurality of pixels of block 282, and (iii) the Pearson's correlation coefficient of block 280 is used to determine stability and turnover rates of proteins. For instance, in such colocalization analysis, the nucleic acid analyte is for the gene and the non-nucleic acid analyte is for the protein gene product of the gene. In some such embodiments such colocalization compare colocalized gene expression (of the gene, nucleic acid analyte) with protein abundance (non-nucleic acid analyte, protein gene product of the gene) to provide insights into the stability and turnover rates of the protein gene product of the gene. Certain proteins might be produced at high mRNA levels, but their abundance might be low due to rapid degradation. Understanding protein turnover, through such colocalization studies, is important for understanding cellular homeostasis and the dynamics of protein regulation.

In some embodiments the first measure of colocalization is used in drug discovery and development. In some embodiments, the first measure of colocalization and the second measure of colocalization is used in drug discovery and development. In some embodiments, the first measure of colocalization, the second measure of colocalization, the ratio between the number of pixels in the first subset of the first plurality of pixels and the number of pixels in the first subset of the second plurality of pixels is used in drug discovery and development. In some embodiments, the first measure of colocalization of block 278, the second measure of colocalization of block 284, the ratio between the number of pixels in the first subset of the first plurality of pixels and the number of pixels in the first subset of the second plurality of pixels of block 282, and the Pearson's correlation coefficient of block 280 is used in drug discovery and development. In some embodiments, the first measure of colocalization of block 278 and any combination of (i) the second measure of colocalization of block 284, (ii) the ratio between the number of pixels in the first subset of the first plurality of pixels and the number of pixels in the first subset of the second plurality of pixels of block 282, and (iii) the Pearson's correlation coefficient of block 280 is used in drug discovery and development. For instance, in any of the forgoing colocalization analysis, through determining which genes (localized by the nucleic acid analyte) are colocalized with disease-related proteins (localized by the non-nucleic acid analyte), potential drug targets and design therapies that modulate the expression or function of these genes and proteins are developed.

Thus, the colocalization of gene expression and protein abundance data through colocalization analysis of the present disclosure enhances the understanding of gene regulation, cellular processes, and disease mechanisms.

Another aspect of the present disclosure provides a computer system comprising one or more processors, memory, and one or more programs, where the one or more programs are stored in the memory and configured to be executed by the one or more processors. The one or more programs include instructions for any of the methods, workflows, processes, or embodiments disclosed herein, and/or any substitutions, modifications, additions, deletions, and/or combinations thereof, as will be apparent to one skilled in the art.

Another aspect of the present disclosure provides a non-transitory computer readable storage medium storing one or more programs, the one or more programs comprising instructions, which when executed by an electronic device with one or more processors and a memory cause the electronic device to perform any of the methods, workflows, processes, or embodiments disclosed herein, and/or any substitutions, modifications, additions, deletions, and/or combinations thereof, as will be apparent to one skilled in the art.

### EXAMPLES

Example 1. Serial (5 μm) sections of FFPE human samples were placed on Visium Gene Expression (GEX) slides. Each capture area incorporates ~5,000 molecularly barcoded, spatially encoded capture spots onto which tissue sections are mounted, stained, and imaged. After imaging, samples were incubated with a human whole transcriptome, probe-based RNA panel followed by an immunology (oligo-tagged) antibody panel. Subsequently, the sections were permeabilized, and the paired gene and protein libraries were generated for each individual section, which were then sequenced on an Illumina NovaSeq at a depth of ~50,000 reads per spot. The resulting reads from both libraries were aligned enabling analysis of both mRNA and protein expression. Additional analyses and data visualization were performed on the 10× Loupe Browser desktop software. In particular the UMI coded, spatially barcoded resulting sequence reads for both the mRNA and protein expression are used to construct the first and second image, respectively. Further details are described in Uytingco et al., “Multiomic characterization of the tumor microenvironment in FFPE tissue by simultaneous protein and gene expression profiling,” Cancer Research, 2022-AACR, which is hereby incorporated by reference.

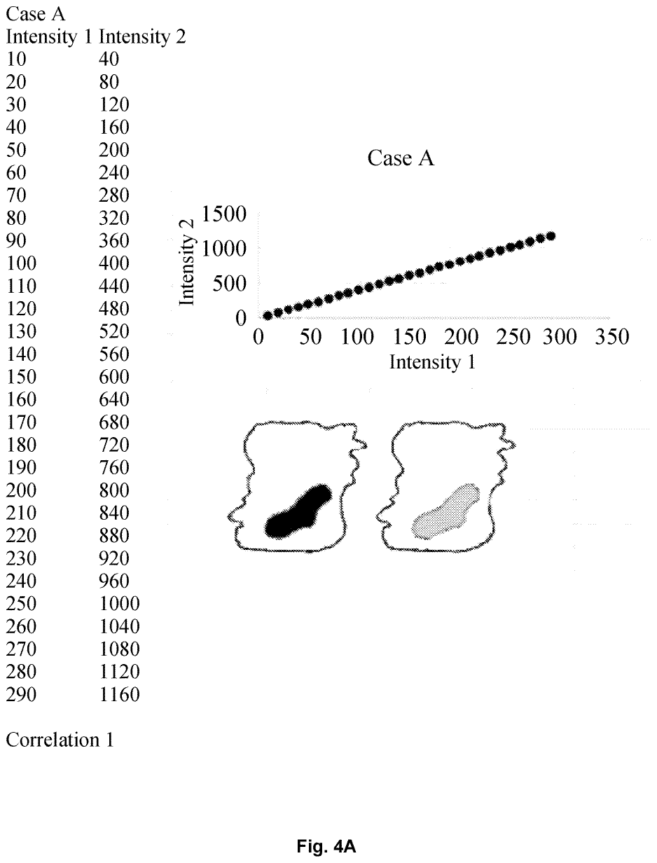

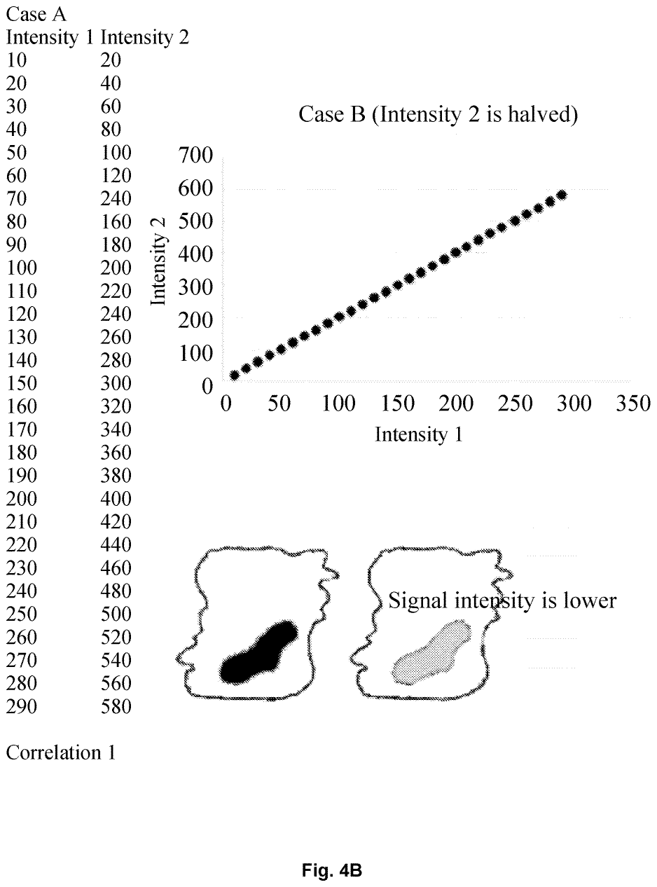

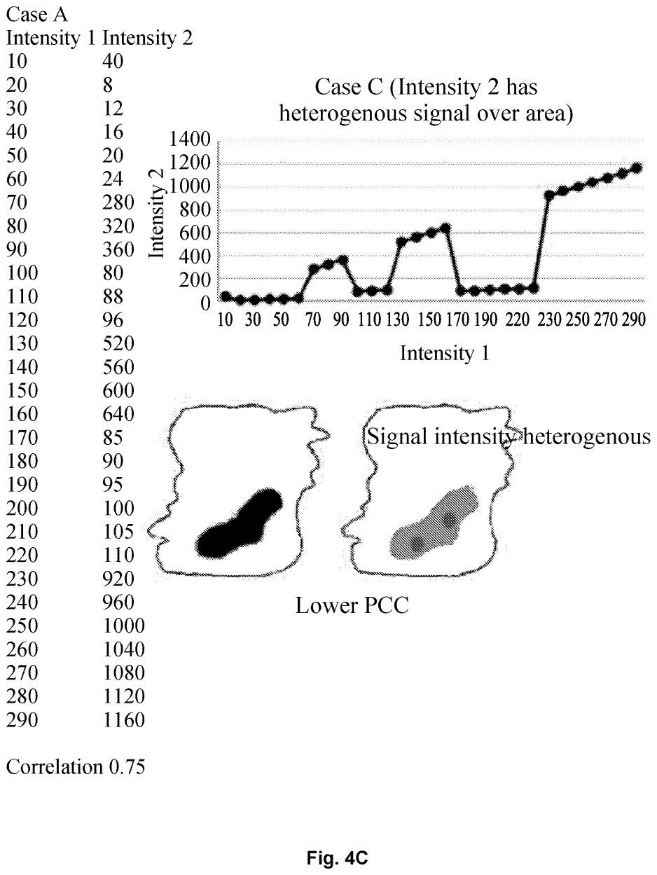

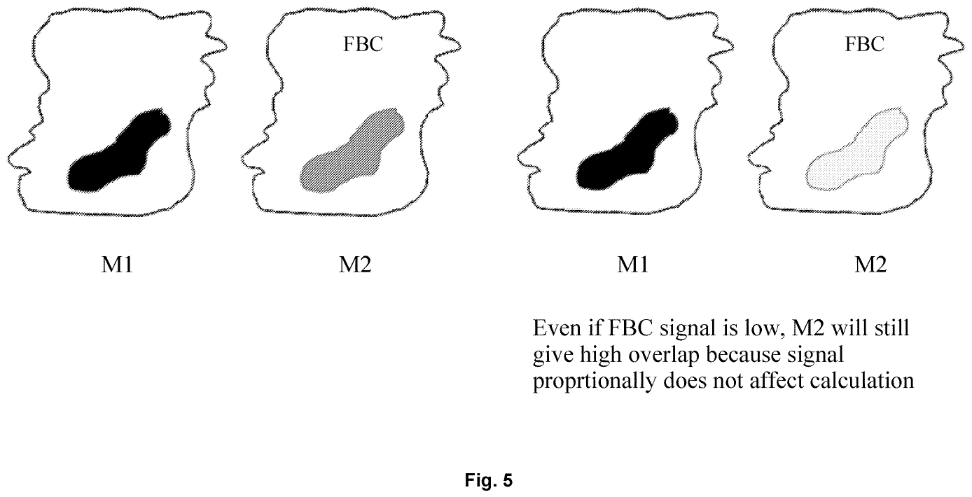

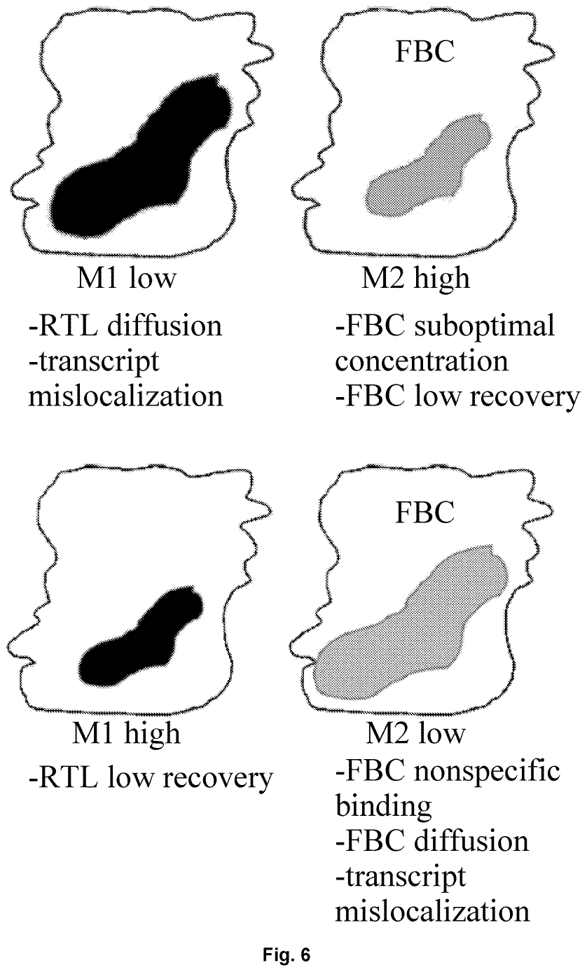

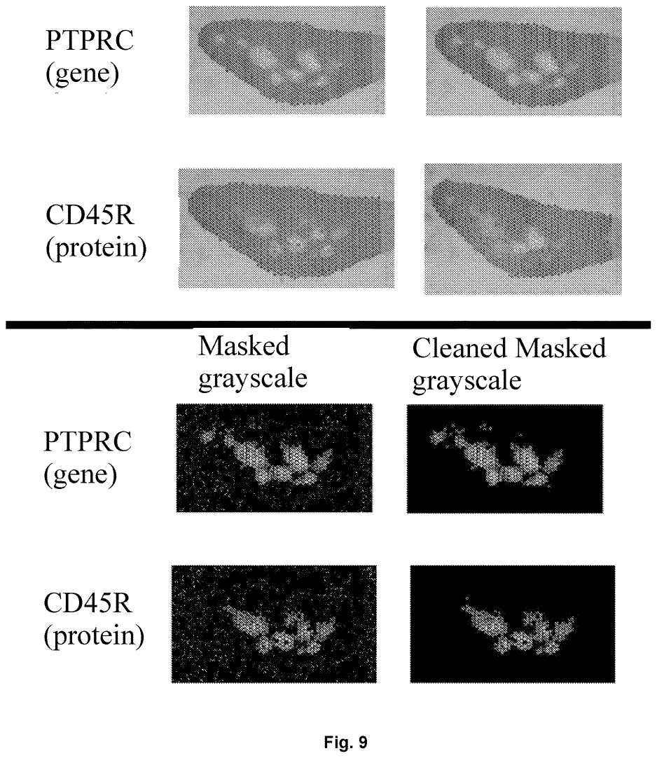

Example 2. FIGS. 4A, 4B, and 4C respectively illustrate three different intensity scenarios between (i) the spatial analyte abundance data for a nucleic acid analyte and (ii) the spatial analyte abundance data for a corresponding non-nucleic acid analyte and how these different scenarios affect the computation of the PCC value in accordance with block 280. In FIG. 4A, the intensity of the two analytes is equal and co-localize, leading to a high PCC value of 1. In FIG. 4B, the intensity of the right-hand analyte is uniformly half the intensity of the left-hand intensity and the two analytes co-localize, leading to a high PCC value of 1. In FIG. 4C, the intensity of the right-hand analyte is heterogeneous with respect to the intensity of the right-hand intensity but the two analytes co-localize. Despite the fact that the two analytes perfectly colocalize in FIG. 4C, they have a lower PCC score because of the heterogeneous intensity of the right-hand analyte with the respect to the left-hand analyte. FIG. 5 illustrates how the drawback of the PCC calculation of block 280 is addressed using the first measure of colocalization of block 278 (M1) and the second measure of colocalization of block 284 (M2). In this instance, even when the feature barcoding (FBC) protein signal (second analyte) is low, as was in the case of the right hand side of FIG. 5 and in FIG. 5C, the M2 (the second measure of colocalization of block 284) will give a high value because signal proportionality does not affect the calculation and the important aspect here is the degree of signal overlap in a given space. As such, the M1 and M2 values for both cases illustrated in FIG. 5 will be the same, as desired, and will be high. FIG. 6 upper panel illustrates a scenario in which M1 (the first measure of colocalization of block 278) is low whereas M2 (the second measure of colocalization of block 284) is high. This scenario could be an indication of suboptimal assay conditions such as FBC concentration used is too low or the FBC recovery is too low. FIG. 6 lower panel illustrates a scenario in which M1 (the first measure of colocalization of block 278) is high whereas M2 (the second measure of colocalization of block 284) is low. This scenario could be an indication of nonspecific FBC binding on the tissue or transcript mislocalization. In FIG. 6, FBC represent the second, or protein, analyte. FIG. 9 illustrates the first (PTPRC gene) and second images (CD45R protein) as obtained from 10× Loupe, after grayscale, after masked greyscale (in accordance with blocks 260 and 262) and cleaned masked greyscale (in accordance with block 234).

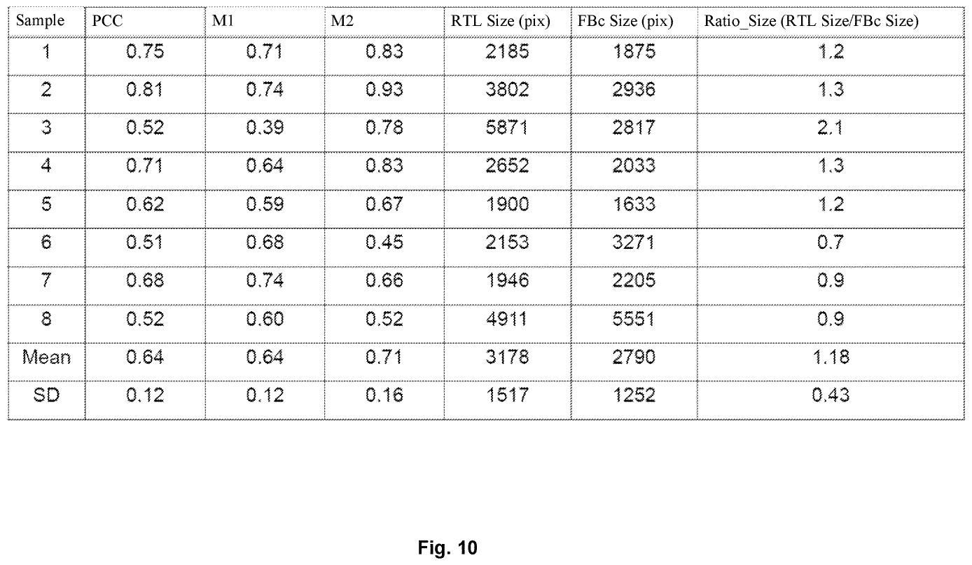

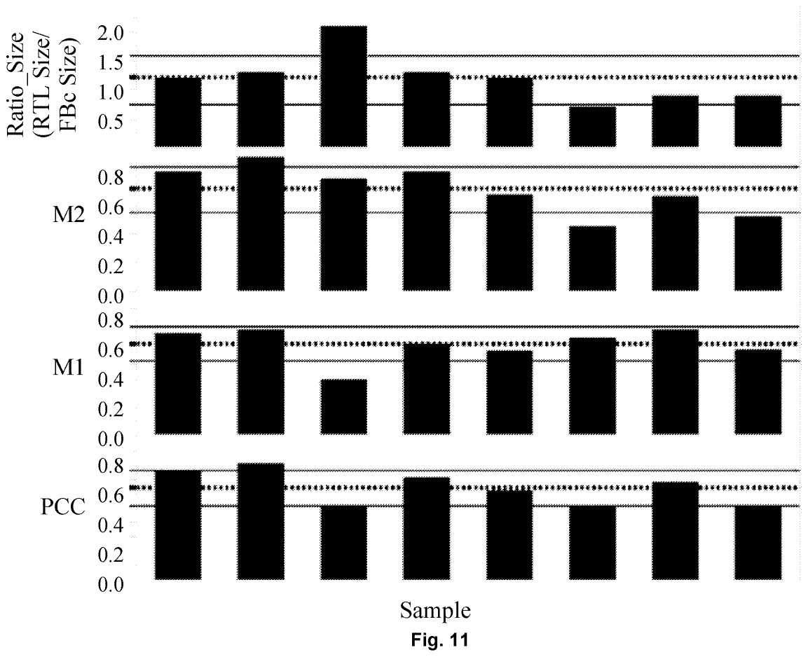

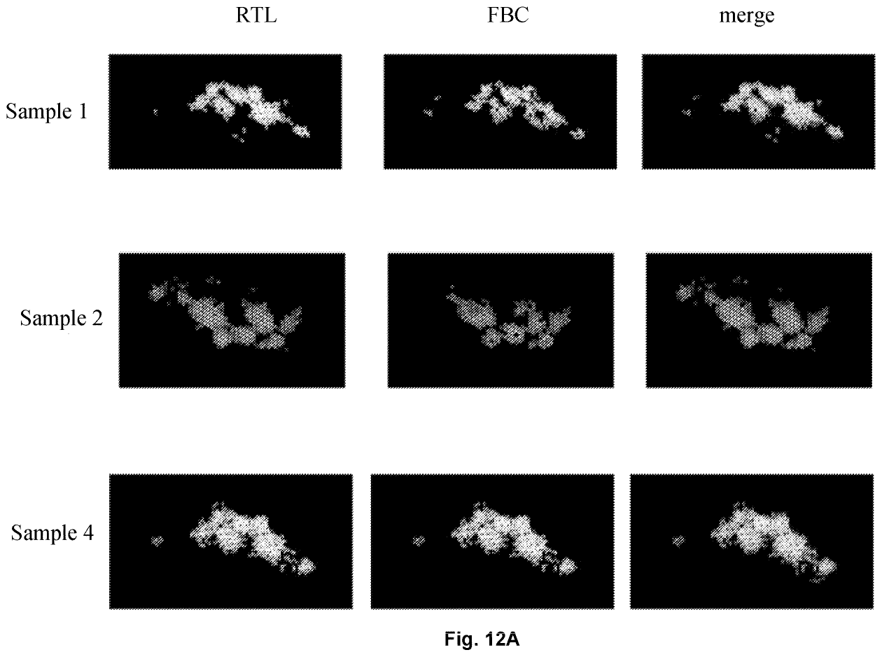

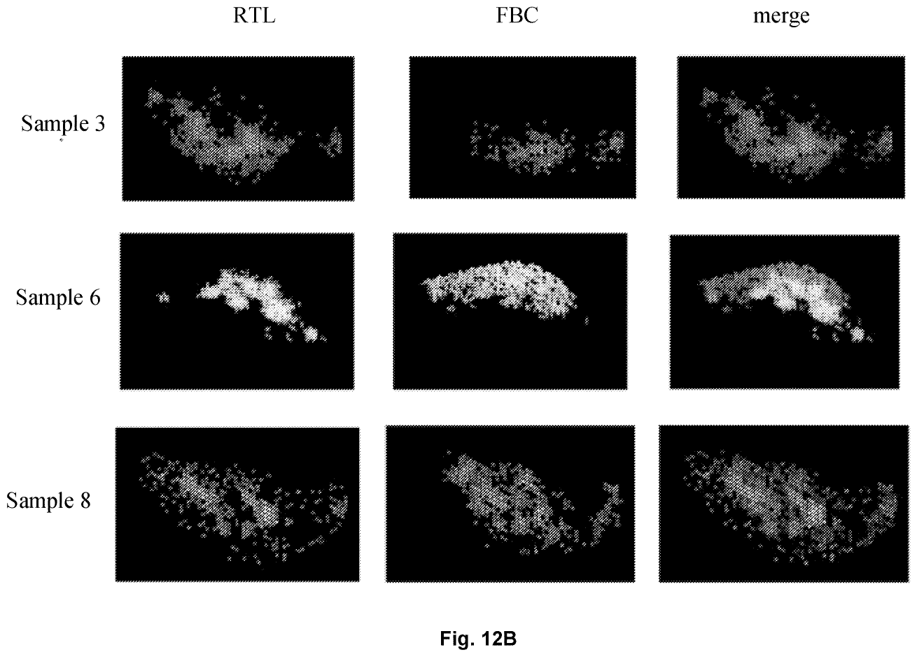

FIG. 10 illustrates PCC (in accordance with block 280), M1 (in accordance with block 278), M2 (in accordance with block 284), number of pixels in the first subset of the first plurality of pixels of the first image (RTL Size), number of pixels in the first subset of the second plurality of pixels of the second image (FBc Size), and ratio (in accordance with block 282) for a nucleic acid analyte and a corresponding non-nucleic acid analyte measured in eight different biological samples. FIG. 11 graphs PCC (in accordance with block 280), M1 (in accordance with block 278), M2 (in accordance with block 284), and the ratio (in accordance with block 282) for the nucleic acid analyte and the corresponding non-nucleic acid analyte measured in the eight different biological samples of FIG. 11. FIGS. 12A and 12B illustrates the RTL (first image, nucleic acid analyte), FBC (second image, non-nucleic acid analyte) and merged first and second image of samples 1, 2 and 4 (FIG. 12A) of FIGS. 10 and 11 which have relatively good overlap, and samples 3, 6, and 8 (FIG. 12B) of FIGS. 10 and 11 which have lower overlap. The metrics given in FIGS. 11 and 12 for M1 and M2 for samples 1-8 show how M1 and M2 provide a good basis for quantitatively distinguishing between the samples of FIGS. 12A and 12B. As such, the example shows that M1 and M2 provide an excellent basis for determining colocalization between (i) the spatial analyte UMI abundance data for a nucleic acid analyte and (ii) the spatial analyte abundance data for a non-nucleic acid analyte.

### REFERENCES CITED AND ALTERNATIVE EMBODIMENTS

All publications, patents, patent applications, and information available on the internet and mentioned in this specification are herein incorporated by reference to the same extent as if each individual publication, patent, patent application, or item of information was specifically and individually indicated to be incorporated by reference. To the extent publications, patents, patent applications, and items of information incorporated by reference contradict the disclosure contained in the specification, the specification is intended to supersede and/or take precedence over any such contradictory material.

The present invention can be implemented as a computer program product that comprises a computer program mechanism embedded in a nontransitory computer readable storage medium. For instance, the computer program product could contain the program modules shown in FIG. 1 and/or described in FIGS. 2A, 2B, 2C, 2D, 2E, 2F, and 2G. These program modules can be stored on a CD-ROM, DVD, magnetic disk storage product, USB key, or any other non-transitory computer readable data or program storage product.

As used herein, the terms “about” or “approximately” refer to an acceptable error range for a particular value as determined by one of ordinary skill in the art, which can depend in part on how the value is measured or determined, e.g., the limitations of the measurement system. For example, “about” can mean within 1 or more than 1 standard deviation, per the practice in the art. “About” can mean a range of ±20%, ±10%, ±5%, or ±1% of a given value. The term “about” or “approximately” can mean within an order of magnitude, within 5-fold, or within 2-fold, of a value. Where particular values are described in the application and claims, unless otherwise stated the term “about” meaning within an acceptable error range for the particular value should be assumed. The term “about” can have the meaning as commonly understood by one of ordinary skill in the art. The term “about” can refer to ±10%. The term “about” can refer to ±5%.

Where values are described in terms of ranges, it should be understood that the description includes the disclosure of all possible sub-ranges within such ranges, as well as specific numerical values that fall within such ranges irrespective of whether a specific numerical value or specific sub-range is expressly stated. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention. As used herein, the term “between” used in a range is intended to include the recited endpoints. For example, a number “between X and Y” can be X, Y, or any value from X to Y.

The term “each,” when used in reference to a collection of items, is intended to identify an individual item in the collection but does not necessarily refer to every item in the collection, unless expressly stated otherwise, or unless the context of the usage clearly indicates otherwise.

As used herein, the singular forms “a,” “an,” and “the” include the plural forms as well, unless the context clearly dictates otherwise. It is further noted that the claims may be drafted to exclude any optional element. As such, this statement is intended to serve as antecedent basis for use of such exclusive terminology as “solely,” “only,” and the like in connection with the recitation of claim elements or use of a “negative” limitation. Furthermore, to the extent that the terms “including,” “includes,” “having,” “has,” “with,” or variants thereof are used in either the detailed description and/or the claims, such terms are intended to be inclusive in a manner similar to the term “comprising.”

Many modifications and variations of this invention can be made without departing from its spirit and scope, as will be apparent to those skilled in the art. The specific embodiments described herein are offered by way of example only. The embodiments were chosen and described in order to best explain the principles of the invention and its practical applications, to thereby enable others skilled in the art to best utilize the invention and various embodiments with various modifications as are suited to the particular use contemplated. The invention is to be limited only by the terms of the appended claims, along with the full scope of equivalents to which such claims are entitled.

What is claimed:

1. A method for colocalization of two analytes in a biological sample, the method comprising:
using a computer system comprising one or more processing cores and a memory:
obtaining, in electronic form, a first two-dimensional image exceeding 100 kilobytes in size, comprising a first plurality of pixels, and wherein the first two-dimensional image encodes spatial analyte unique molecular identifier (UMI) abundance data for a nucleic acid analyte from the biological sample from a subject through pixel intensity values across a first subset of pixels in the first plurality of pixels, wherein the first subset of pixels in the first plurality of pixels comprises 500 pixels;
obtaining, in electronic form, a second two-dimensional image exceeding 100 kilobytes in size, comprising a second plurality of pixels, and wherein the second two-dimensional image encodes spatial analyte abundance data for a non-nucleic acid analyte from the biological sample through pixel intensity values across a first subset of pixels in the second plurality of pixels, wherein the first subset of the second plurality of pixels comprises 500 pixels and wherein a registration between the first and second image is determined;
obtaining a first threshold value of the first image;
obtaining a second threshold value of the second image;
determining, using the registration, a first summation of the respective UMI abundance value of each respective pixel in the first subset of the first plurality of pixels in which the corresponding pixel in the first subset of the second plurality of pixels exceeds the second threshold value; and
providing the first summation, normalized to a second summation of each respective UMI abundance value of each pixel in the first subset of the first plurality of pixels, as a first measure of colocalization between (i) the spatial analyte UMI abundance data for the nucleic acid analyte and (ii) the spatial analyte abundance data for the non-nucleic acid analyte.

2. The method of claim 1, the method further comprising:
determining, using the registration, a third summation of the respective UMI abundance value of each respective pixel in the first subset of the second plurality of pixels in which the corresponding pixel in the first subset of the first plurality of pixels exceeds the first threshold value; and
providing the third summation, normalized to a fourth summation of each respective abundance value of each pixel in the first subset of the second plurality of pixels, as a second measure of colocalization between (i) the spatial analyte UMI abundance data for the nucleic acid analyte and (ii) the spatial analyte abundance data for the non-nucleic acid analyte.

3. The method of claim 1 or 2, wherein the nucleic acid analyte is a plurality of RNA or DNA molecules, arising from the biological sample, wherein
each RNA or DNA molecule in the plurality of RNA or DNA molecules encodes all or a portion of a first gene, and
the spatial analyte UMI abundance data for the nucleic acid analyte quantifies UMI abundance of a plurality of spatially barcoded sequence reads of the plurality of RNA or DNA molecules.

4. The method of claim 3, wherein the non-nucleic acid analyte is a first protein and the spatial analyte abundance data for the non-nucleic acid analyte quantifies abundance of a labeled antibody to the first protein.

5. The method of claim 4, wherein the first gene encodes the first protein.

6. The method of any one of claims 1-5, the method further comprising:
segmenting the first image to identify the first subset of the first plurality of pixels using a first segmentation algorithm; and
segmenting the second image to identify the first subset of the second plurality of pixels using a second segmentation algorithm.

7. The method of claim 6, wherein the first segmentation algorithm and the second segmentation algorithm is a binarization method using global thresholding.

8. The method of claim 6, wherein the first segmentation algorithm identifies the first threshold value as a first pixel intensity value that that divides the pixels of the first image into either the first subset of the first plurality of pixels or a second subset of the first plurality of pixels, wherein the first threshold value represents a minimization of intra-class intensity variance between the first and second subset of the first plurality of pixels or a maximization of inter-class variance between the first and second subset of the first plurality of pixels.

9. The method of any one of claims 1-8, the method further comprising:
determining a Pearson's correlation coefficient using (i) the intensity of each pixel in the first subset of the first plurality of pixels, (ii) the intensity of each pixel in the second image corresponding to a pixel in the first subset of the first plurality of pixels, (iii) a first mean value across the first subset of the first plurality of pixels, and (iv) a second mean value across the pixels in the second image corresponding to the first subset of pixels in the first plurality of pixels; and
providing the Pearson's correlation coefficient as a third measure of colocalization between (i) the spatial analyte UMI abundance data for the nucleic acid analyte and (ii) the spatial analyte abundance data for the non-nucleic acid analyte.

10. The method of any one of claims 1-9, the method further comprising:
determining a ratio between a number of pixels in the first subset of the first plurality of pixels and a number of pixels in the first subset of the second plurality of pixels; and
providing the ratio as an indication of a size comparison between (i) the spatial analyte UMI abundance data for the nucleic acid analyte and (ii) the spatial analyte abundance data for the non-nucleic acid analyte.

11. The method of any one of claims 1-10, the method further comprising:
displaying the first image on a display in electronic communication with the computer system;
obtaining user selection of a first region of the first image, wherein each pixel in the first subset of pixels in the first plurality of pixels is within the first region; and
removing from the first image all pixels outside the first region prior to obtaining the threshold value of the first image.

12. The method of claim 11, the method further comprising:
displaying the second image on a display in electronic communication with the computer system;
obtaining user selection of a second region of the second image, wherein each pixel in the first subset of pixels in the second plurality of pixels is within the second region; and
removing from the second image all pixels outside the second region prior to obtaining the threshold value of the second image.

13. The method of any one of claims 1-12, the method further comprising:
removing, from the first subset of the first plurality of pixels, clusters of pixels within the first image that constitute less than a threshold number of pixels; and
removing, from the first subset of the second plurality of pixels, clusters of pixels within the second image that constitute less than a threshold number of pixels.

14. The method of claim 13, wherein the threshold number of pixels is 10 pixels.

15. The method of any one of claims 1-14, wherein
the first subset of the first plurality of pixels occupies a first plurality of disjoint areas within the first image; and
the first subset of the second plurality of pixels occupies a second plurality of disjoint areas within the second image.

16. The method of claim 15, wherein
the first plurality of disjoint areas comprises five disjoint areas; and
the second plurality of disjoint areas comprises five disjoint areas.

17. The method of any one of claims 1-16, wherein the intensity of the first plurality of pixels and the second plurality of pixels is on a log₂ based scale relative to measured intensity.

18. The method of any one of claims 1-17, the method further comprising downsampling the first image to a size of the second image prior to determining, using the registration, the first summation.

19. The method of any one of claims 1-17, the method further comprising downsampling the second image to a size of the first image prior to determining, using the registration, the first summation.

20. The method of any one of claims 1-19, wherein the second image is obtained by bright-field microscopy, immunohistochemistry, or fluorescence microscopy.

21. The method of any one of claims 1-20, wherein the biological sample is prepared for imaging to create the second image using a detectable marker selected from the group consisting of an antibody, a fluorescent label, a radioactive label, a chemiluminescent label, a colorimetric label, a colorimetric label, or a combination thereof.

22. The method of any one of claims 1-20, wherein the biological sample is prepared for imaging to create the second image using a stain selected from the group consisting of live/dead stain, trypan blue, periodic acid-Schiff reaction stain, Masson's trichrome, Alcian blue, van Gieson, reticulin, Azan, Giemsa, Toluidine blue, isamin blue, sudan black and osmium, acridine orange, Bismarck brown, carmine, Coomassie blue, cresyl violet, DAPI, eosin, ethidium bromide, acid fuchsine, hematoxylin, Hoechst stains, iodine, methyl green, methylene blue, neutral red, Nile blue, Nile red, osmium tetroxide, propidium iodide, rhodamine, safranin, or a combination thereof.

23. The method of any one of claims 1-22, wherein the first image is obtained by filtering a spatial dataset for UMI abundance data for the nucleic acid analyte, and wherein the method further comprises obtaining the spatial dataset by a procedure comprising:
overlaying the biological sample on a substrate, wherein the substrate comprises a set of capture spots in the form of an array;
obtaining a plurality of sequence reads, in electronic form, from the set of capture spots, wherein:
each respective capture probe plurality in a set of capture probe pluralities is (i) at a different capture spot in the set of capture spots and (ii) directly or indirectly associates with one or more nucleic acid analytes in a plurality of nucleic acid analytes from the biological sample,
each respective capture probe plurality in the set of capture probe pluralities is characterized by at least one unique spatial barcode in a plurality of spatial barcodes,
the plurality of sequence reads comprises sequence reads corresponding to all or portions of the plurality of nucleic acid analytes,
the plurality of sequence reads comprises at least 10,000 sequence reads, and
each respective sequence read in the plurality of sequence reads includes a spatial barcode of the corresponding capture probe plurality in the set of capture probe pluralities or a complement thereof; and
using all or a subset of the plurality of spatial barcodes to localize respective sequence reads in the plurality of sequence reads to corresponding capture spots in the set of capture spots, thereby dividing the plurality of sequence reads into a plurality of subsets of sequence reads, each respective subset of sequence reads corresponding to a different capture spot in the plurality of capture spots.

24. The method of claim 23, wherein the unique spatial barcode encodes a unique predetermined value selected from the set {1, . . . , 1024}, {1, . . . , 4096}, {1, . . . , 16384}, {1, . . . , 65536}, {1, . . . , 262144}, {1, . . . , 1048576}, {1, . . . , 4194304}, {1, . . . , 16777216}, {1, . . . , 67108864}, or {1, . . . , 1×10^{12}.}

25. The method of claim 23 or 24, wherein the obtaining the plurality of sequence reads comprises high-throughput sequencing.

26. The method of any one of claims 23 through 25, wherein a respective capture probe plurality in the set of capture probe pluralities includes 1000 or more capture probes, 2000 or more capture probes, 10,000 or more capture probes, 100,000 or more capture probes, 1×10⁶ or more capture probes, 2×10⁶ or more capture probes, or 5×10⁶ or more capture probes.

27. The method of claim 26, wherein each capture probe in the respective capture probe plurality includes the same spatial barcode from the plurality of spatial barcodes.

28. The method of any one of claims 1-27, wherein the biological sample is a tissue section.

29. The method of any one of claims 1-28, further comprising using the first measure of colocalization to characterize a biological condition in the subject.

30. The method of any one of claims 1-29, wherein
the first image comprises 10,000 or more pixel values, and
the second image comprises 10,000 or more pixel values.

31. The method of any one of claims 1-29, wherein
the first image comprises 100,000 or more pixel values, and
the second image comprises 100,000 or more pixel values.

32. The method of any one of claims 1-30, wherein the first summation provides an extent to which (i) the spatial analyte UMI abundance data for the nucleic acid analyte and (ii) the spatial analyte abundance data for the non-nucleic acid analyte are colocalized in the biological sample.

33. The method of any one of claims 1-30, wherein the first summation provides an extent to which (i) the spatial analyte UMI abundance data for the nucleic acid analyte and (ii) the spatial analyte abundance data for the non-nucleic acid analyte colocalize to a common component of a plurality of cells within the biological sample.

34. The method of claim 33, wherein the common component is a cell membrane, cytoplasm, a nucleus, a nuclear envelope, a nucleolus, an endoplasmic reticulum, a ribosome, a Golgi apparatus, a mitochondria, a lysosome, a vacuole, a peroxisome, a cytoskeleton, or a centriole.

35. The method of any one of claims 1-34, wherein the first summation provides an identification of a function of a protein.

36. The method of any one of claims 1-34, wherein the first summation provides an association of a protein with a particular biological pathway.

37. A computer system comprising
one or more processors;
a memory; and
one or more programs, wherein the one or more programs are stored in the memory and configured to be executed by the one or more processors, the one or more programs including instructions for performing the method for colocalization of any one of claims 1-36.

38. A computer system comprising:
one or more processors;
memory; and
one or more programs, wherein the one or more programs are stored in the memory and configured to be executed by the one or more processors, the one or more programs including instructions for performing a method for colocalization comprising:
obtaining, in electronic form, a first two-dimensional image exceeding 100 kilobytes in size, comprising a first plurality of pixels, and wherein the first two-dimensional image encodes spatial analyte unique molecular identifier (UMI) abundance data for a nucleic acid analyte from a biological sample from a subject through pixel intensity values across a first subset of pixels in the first plurality of pixels, wherein the first subset of the first plurality of pixels comprises 500 pixels;
obtaining, in electronic form, a second two-dimensional image exceeding 100 kilobytes in size, comprising a second plurality of pixels, and wherein the second two-dimensional image encodes spatial analyte abundance data for a non-nucleic acid analyte from the biological sample through pixel intensity values across a first subset of pixels in the second plurality of pixels, wherein the first subset of the second plurality of pixels comprises 500 pixels and a registration between the first and second image is determined;
obtaining a first threshold value of the first image;
obtaining a second threshold value of the second image;
determining, using the registration, a first summation of the respective UMI abundance value of each respective pixel in the first subset of the first plurality of pixels in which the corresponding pixel in the first subset of the second plurality of pixels exceeds the second threshold value; and
providing the first summation, normalized to a second summation of each respective UMI abundance value of each pixel in the first subset of the first plurality of pixels, as a first measure of colocalization between (i) the spatial analyte UMI abundance data for the nucleic acid analyte and (ii) the spatial analyte abundance data for the non-nucleic acid analyte.

39. A computer readable storage medium storing one or more programs, the one or more programs comprising instructions, which when executed by an electronic device with one or more processors and a memory cause the electronic device to perform a method for colocalization comprising:
obtaining, in electronic form, a first two-dimensional image exceeding 100 kilobytes in size, comprising a first plurality of pixels, and wherein the first two-dimensional image encodes spatial analyte unique molecular identifier (UMI) abundance data for a nucleic acid analyte from a biological sample of a subject through pixel intensity values across a first subset of pixels in the first plurality of pixels, wherein the first subset of the first plurality of pixels comprises 500 pixels;
obtaining, in electronic form, a second two-dimensional image exceeding 100 kilobytes in size, comprising a second plurality of pixels, and wherein the second two-dimensional image encodes spatial analyte abundance data for a non-nucleic acid analyte from the biological sample through pixel intensity values across a first subset of pixels in the second plurality of pixels, wherein the first subset of the second plurality of pixels comprises 500 pixels and a registration between the first and second image is determined;
obtaining a first threshold value of the first image;
obtaining a second threshold value of the second image;
determining, using the registration, a first summation of the respective UMI abundance value of each respective pixel in the first subset of the first plurality of pixels in which the corresponding pixel in the first subset of the second plurality of pixels exceeds the second threshold value; and
providing the first summation, normalized to a second summation of each respective UMI abundance value of each pixel in the first subset of the first plurality of pixels, as a first measure of colocalization between (i) the spatial analyte UMI abundance data for the nucleic acid analyte and (ii) the spatial analyte abundance data for the non-nucleic acid analyte.

40. A computer readable storage medium storing one or more programs, the one or more programs comprising instructions, which when executed by an electronic device with one or more processors and a memory cause the electronic device to perform the method for colocalization of any one of claims 1-36.
